(19) United States
(12) Patent Application Publication
(10) Pub. No.: US 2025/0288695 A1
(43) Pub. Date: Sep. 18, 2025

(54) TSPAN2 AS SCAFFOLD PROTEIN FOR ENGINEERED EXTRACELLULAR VESICLES

(71) Applicant: Evox Therapeutics Ltd, Oxford Oxfordshire (GB)

(72) Inventors: Xiuming Liang, Huddinge (SE); Dhanu Gupta, Huddinge (SE); Joel Nordin, Huddinge (SE); Samir EI-Andaloussi, Huddinge (SE); Wenyi Zheng, Huddinge (SE); Zheyu Niu, Huddinge (SE); Julia Anna Radler, Huddinge (SE)

(21) Appl. No.: 18/862,248

(22) PCT Filed: May 4, 2023
(86) PCT No.: PCT/EP2023/061871

§ 371 Date: Nov. 1, 2024

(30) Foreign Application Priority Data
May 6, 2022 (GB) 2206625.2

(51) Int. Cl. A61K 48/00; A61K 47/64; C07K 14/705; C12N 15/88

(52) U.S. Cl. CPC A61K 48/0058; A61K 47/6425; C07K 14/705; C12N 15/88; C07K 2319/01

(57) ABSTRACT

The present invention pertains to a genetically engineered extracellular vesicle comprising an improved scaffold protein and a protein of interest. Polypeptide constructs, polynucleotide constructs, cells, pharmaceutical compositions and methods relating to the same are also disclosed.

### REFERENCE TO AN ELECTRONIC SEQUENCE LISTING

The contents of the electronic sequence listing (EVOX_028_001WO_SeqList_ST26.xml; Size: 8,927 bytes; and Date of Creation: Apr. 28, 2023) are herein incorporated by reference in its entirety.

### TECHNICAL FIELD

The present invention relates to a newly identified extracellular vesicle (EV) scaffold protein, which is advantageously used for genetically engineered extracellular vesicles. The present invention also relates to uses of said genetically engineered EVs in therapy, methods of production and purification of said EVs, populations of said EVs as well as polypeptide and polynucleotide constructs encoding the new scaffold protein and cells comprising those polypeptide or polynucleotide constructs.

### BACKGROUND ART

EVs (such as exosomes) are typically nanometer-sized vesicles produced by most cell types and functioning as the body's natural transport system for proteins, nucleic acids, peptides, lipids, and various other molecules between cells. EVs have a number of potential therapeutic uses and EVs are promising natural delivery vehicles for protein, nucleic acid and small molecule therapeutics. Engineering of EVs to comprise proteins of interest (POIs) is critical to the utility of EVs as a delivery of therapeutic cargos; however, identifying suitable EV proteins to act as fusion partners to transport therapeutic cargos into EVs has proven difficult and unpredictable.

Loading therapeutic molecules of interest can be achieved in multiple ways. In the simplest scenario, small EVs from producer cells with intrinsic therapeutic potentials, such as immunomodulatory mesenchymal stem cells and chimeric antigen receptor-T cells, are passively loaded with therapeutic molecules and can thus be directly deployed for disease management. Alternatively, exogenous loading of therapeutic molecules using physical and chemical methods like incubation, sonication, and electroporation have been widely applied. This strategy is, however, limited to small RNAs (e.g., siRNA, miRNA, shRNA) and low molecular weight chemicals and at considerable risk for technical artifacts such as RNA precipitation. POIs, on the other hand, are usually loaded into small EVs through genetic engineering. In a typical workflow, producer cells are engineered to overexpress the desirable protein fused to an EV-sorting/carrier domain, which promotes endogenous sorting of the POI into EVs during their biogenesis. Moreover, potential RNA therapeutics can be indirectly loaded via the same strategy by using compatible RNA-binding proteins.

Dependent on the topology and subcellular location of the EV protein used to carry the POI into the engineered EVs (the so-called “scaffold domain” or “scaffold protein”), as well as the terminal used for fusion, the cargo molecule can either be displayed on the EV surface or loaded into the vesicle. In contrast to surface display, intravesicular loading protects the cargo molecule from premature release as well as rapid degradation. Previously reported EV scaffold domains for intravesicular loading fall into three major categories: cytoplasmic proteins (SDCB-1, ARRDC1 and BASP1), single-pass transmembrane proteins (LAMP2B and PTGFRN), and four-pass transmembrane proteins (CD9, CD63, and CD81).

Traditionally “common” or “classical” EV proteins, which are highly expressed natively in EVs, were considered as the best EV proteins to use as “scaffolds” to carry the POIs into the engineered EVs. Generation of fusion proteins of EV protein-POI has been shown to be an effective way to load an EV with a chosen POI. As above, the use of classical EV proteins such as CD63, CD9 and CD81 as well as single pass transmembrane EV proteins such as Lamp2 or even membrane associated EV proteins such as BASP as the fusion partner for a POI have previously been demonstrated.

However, the EV-sorting efficiency of the above-mentioned domains is contingent on the ability of producer cells to consistently express a given scaffold domain and the specific type and size of cargo molecules which are carried by the domain, which has previously caused highly variable outcomes.

For example, expression of the transgene, CD63-POI, is known to decline significantly over time in the producer cell compromising the yield of EV product. In other words, when using CD63 as the EV scaffold protein, even after generating a stable cell line, the stability of that cell line has been shown to be very variable and decline over time, so that there are fewer CD63-POI fusion constructs on any one EV and also fewer EVs comprising any transgene fusion protein construct.

In addition, when CD63 is engineered so it is fused to comprise more than one POI (e.g. with different POIs fused to the N terminus, C-terminus, 1st loop and/or 2nd loop), the number of EVs expressing the CD63 fusion construct and the number of CD63 fusion construct expressed per EV is reduced.

There consequently remains a need for improved ways of introducing POIs into EVs.

### SUMMARY OF INVENTION

In addition to the foregoing known limitation, it has also become apparent to the present inventors that using these classical EV proteins is problematic when additional proteins or fusion protein constructs are required to be added to the therapeutic EV. For example, the creation of double stable cell lines that consistently express more than one POI, which are to be expressed on or within the same EV population, is not possible. The present inventors have found that, if a double stable cell line was produced using a “classical EV protein” such as CD63, CD9 or CD81, then very quickly the expression of the second construct would decline (for example, CD63-POI being co-expressed with another POI such as VSVG would rapidly result in the VSVG expression being abrogated); the result being that the population of EVs produced would rapidly only comprise the protein construct comprising the “classical EV protein”. This is of course problematic when a therapeutic EV is required to comprise more than one POI because production of a consistent EV producer cell line is not possible. The reason for this is unknown.

Thus, whilst it has been found that classical EV proteins such as CD63, CD81 and CD9 are highly expressed on native EVs/exosomes when engineered to comprise a POI, it has also been found that they prevent the introduction of subsequent (different) proteins into the same EV and thus prevent co-localisation of multiple protein constructs on the same EV. This seriously restricts the combinations of proteins that can be introduced into EVs, so prohibiting any multiplexing of functionality. It also limits the development of therapeutic EVs as a platform technology and significantly reduces the efficacy of the engineered EVs

In addition, whilst classical tetraspanin EV proteins, such as CD63, may theoretically be engineered to comprise multiple POIs (i.e. on the N terminus, C-terminus, 1^{st} loop and/or 2^{nd} loop), it has been found that engineering such classical tetraspanin EV proteins, so they are fused to comprise more than one POI (e.g. with different POIs fused to the N terminus, C-terminus, 1^{st} loop and/or 2^{nd} loop), results in the number of EVs expressing these fusion construct and the number of these fusion constructs expressed per EV is reduced.

There consequently also remains a need for new ways of introducing multiple POIs into EVs.

To address these limitations and cope with future challenges for genetic engineering, there is a demand to identify new EV-sorting domains. The present invention was therefore born from a study designed to discover novel EV-sorting domains and investigate differences between several producer cells and cargo molecules.

It is an object of the present invention to overcome the above-identified problems associated with finding novel EV carrier/scaffold proteins that can be used as a fusion partner to transport POIs into EVs which high efficiency and reliability over an extended time period. The present inventors desired to identify novel EV carrier/scaffold proteins that would act as “universal EV carrier/scaffold proteins”, i.e. that are effective across numerous cell types and capable of carrying a wide range of cargos.

Secondly, it is an object of the present invention to identify EV scaffold/carrier proteins that maintain expression of the EV-protein-POI fusion construct and do not prevent co-localization of a second or subsequent POIs on the same EV. The present inventors sought to identify an alternative scaffold protein which can also be engineered to include a POI whilst at the same time allowing a second or further proteins to be engineered into/onto the same EV, i.e. allowing co-localisation.

In addition, the present inventors sought to identify EV scaffold/carrier proteins that can be engineered to comprise multiple POIs and which, when engineered in this manner, continue to be expressed at a high level in EVs.

Further, the present inventors are always seeking to identify scaffold proteins that enable increased loading of cargos, including RNAs, into EVs.

To these ends the present inventors conducted a large scale screen on EV proteins and identified TSPAN2 as a novel and highly flexible scaffold protein, which unlike classical EV proteins, allows the development of a highly flexible and therefore versatile platform technology. TSPAN2 shows remarkably improved (up to 10 fold improved) function as an EV scaffold protein as compared to classical EV proteins and by enabling colocalization of other protein constructs in the same EV this significantly increases the modularity of this therapeutic exosome platform technology.

In addition, both TSPAN2 and TSPAN3 allow for increased mRNA loading, as compared to the classical EV tetraspanin, CD63. Furthermore, expression of TSPAN2 fused to a POI results in an increased proportion of the EVs produced expressing the fusion construct, as well as an increase in the number of these fusion constructs expressed per EV, as compared with expression of classical EV tetraspanin CD63 fused to a POI. This is also the case where TSPAN2 is fused to multiple POIs.

Thus, in a first aspect the present invention provides an EV comprising a fusion protein, wherein the fusion protein comprises TSPAN2 fused to a POI.

In second and third aspects the present invention provides a polypeptide construct comprising TSPAN2 fused to a POI as well as a polynucleotide construct encoding such a polypeptide construct.

In a fourth aspect the present invention provides a cell comprising the polypeptide construct of the second aspect or the polynucleotide construct of the third aspect. Optionally the cells may further comprise a second polypeptide construct or polynucleotide construct capable of expressing a POI.

In a fifth aspect the present invention provides a pharmaceutical composition comprising an EV of the first aspect or a cell of the fourth aspect and a pharmaceutically acceptable excipient or carrier.

In a sixth aspect the present invention provides methods for producing the EVs of the first aspect. Said method for producing EVs comprises: (i) introducing into an EV-producing cell a polynucleotide construct encoding a TSPAN2-POI fusion construct; and (ii) expressing the construct in the EV-producing cell, thereby generating EVs comprising the TSPAN2-POI fusion protein. The method may optionally further comprise an intermediate step of introducing into the same EV producing cell a second polynucleotide construct encoding a second POI (optionally wherein the second POI is present in the form of a fusion protein with an classical EV protein) before continuing to express both constructs in the EV-producing cell, thereby generating EVs comprising the TSPAN2-POI fusion protein and the second POI.

In a seventh aspect the present invention provides EVs of the first aspect for use in therapy. In an eighth aspect the present invention also provides a method of treatment comprising administering to a patient in need thereof, an effective amount of EVs according to the first aspect of invention or a pharmaceutical composition of the fifth aspect.

It is shall be clear from the present Examples that the discovery of TSPAN2 as a novel scaffold protein would not have been possible without the large scale screening method presently described and the significant inventive efforts of the present inventors.

### BRIEF DESCRIPTION OF FIGURES

FIG. 1. Schematic showing high-throughput screening scheme and results.

FIG. 2. Results of screening for ThermoLuc (Tluc) in the secretome of HEK-293T cells. (A-E showing correlations indicating the reliability of the screening protocol and identifying top 36 candidates).

FIG. 3. Results of further screen to assess ability of candidate EV scaffold proteins to sort to small extracellular vesicles.

FIG. 4. Quantification of numbers of engineered EVs produced using different EV scaffold candidates, using bifunctional bioluminescence and fluorescence reporters showing that TSPAN2 and TSPAN3 produce 3.5 fold more engineered EVs than CD63.

FIG. 5. Assessment of top candidates in range of different EV producer cell lines.

FIG. 6. Characterization of TSPAN2 and TSPAN3-engineered EVs by nanoparticle tracking analysis (NTA), western blot, cryoEM, Confocal microscope and quantification of in-vivo bio distribution of Tluc-labeled EVs.

FIG. 7: schematic describing Co-culture vs EV adding systems used to screen for functional activity of novel EV scaffold proteins.

FIG. 8: Scaffold screening results.

FIG. 9: Summary of screening results shown as heat maps.

FIG. 10: Further validation of top 13 EV protein screening candidates using other hard-to-transfect reporter cells (mesenchymal stromal cells (MSC), THP1 Raw264.7 and K562 cells).

FIG. 11: Chart showing comparison of TSPAN2 vs classical EV scaffold proteins to assess ability to allow co-expression of multiple constructs within the same EV.

FIG. 12: Cell sorting data confirming ability of TSPAN2 to allow co-localization of two constructs on the same EV.

FIG. 13: In-vivo data using B16F10 tumor model for the Cre protein delivery using TSPAN2 engineered EVs showing that TSPAN2 is capable of delivering functional POIs.

FIG. 14: TSPAN2-Albumin-binding domain fusion EVs showing albumin binding functionality in-vitro and improved circulatory half-life of TSPAN2 engineered EVs in-vivo. Further confirmation that TSPAN2 is capable of delivering functional POIs.

FIG. 15: Data showing TSPAN2 is able to deliver nucleic acid cargos (Ago2 RNA-binding protein as the POI loading shRNA cargo)

FIG. 16: Data showing TSPAN2 is able to deliver mRNA by attachment to an nucleic acid binding protein.

FIG. 17: Data showing TSPAN2 and TSPAN3 are able to deliver mRNA by attachment to a nucleic acid binding protein.

FIGS. 18 & 19: Data showing TSPAN2 is able to improve the efficiency of EV engineering compared to classical EV scaffold proteins in different cell lines and with different modifications to the scaffold protein.

FIG. 20: Data showing the stability of TSPAN2 constructs over time in EV producer cells.

FIG. 21: Data showing the stability of TSPAN2 constructs over time in EVs secreted by the producer cells.

### BRIEF DESCRIPTION OF SEQUENCE LISTING

SEQ ID NO:1 TSPAN2 protein
SEQ ID NO:2 TSPAN2 nucleic acid
SEQ ID NO:3 TSPAN3 protein
SEQ ID NO:4 TSPAN3 nucleic acid
SEQ ID NO:5 TSPAN18 protein
SEQ ID NO:6 TSPAN18 nucleic acid

### DETAILED DESCRIPTION OF THE INVENTION

The present invention relates to EVs comprising a fusion protein, wherein the fusion protein comprises TSPAN2 fused to a POI. The present invention also relates to cells capable of producing those EVs, methods of making and purifying those EVs and their use in therapy. The EVs of the present invention have a number of distinct advantages due to the use of TSPAN2 in the fusion protein.

Principally the use of TSPAN2 enables improved stability of cell lines producing engineered EVs as well as higher levels of expression of TSPAN2-POI constructs on or within EVs. Furthermore the use of TSPAN2 enables colocalization of other protein constructs in the same EV solving the problem of generating cells lines that stably express more than one fusion construct and this significantly increases the modularity of this therapeutic exosome platform technology.

The present inventors have shown that TSPAN2 is a superior scaffold protein compared to classical scaffold proteins because it displays consistently high expression across a wide range of cell sources, it allows a large range of different types of POIs to be consistently expressed demonstrating up to 10-fold better expression, and EVs expressing TSPAN2 as the scaffold protein also demonstrate better bioavailability as compared to those expressing classical EV proteins. TSPAN2 has been surprisingly found to be an extremely tractable scaffold protein, allowing significant engineering without any of the problems of expression or colocalization observed with classical EV protein scaffolds. TSPAN2 consistently performed the best out of a very large scaffold screen. Importantly only 36 out of 244 candidates were found to be effective indicating the highly heterogeneous EV sorting activity even amongst closely related proteins. As will be seen from the Examples provided herein, the present inventors discovered using a novel functional screening approach that there is poor correlation between rank encapsulation and total secreted reporter protein indicating that the results achieved here are highly unpredictable.

In a first aspect, the invention provides an EV comprising a fusion protein, wherein the fusion protein comprises TSPAN2 fused to a POI.

In one embodiment the POI is engineered into loop 1, loop 2 or both loops of TSPAN2. Advantageously this allows the POI to be displayed on the surface of the EV.

In another embodiment the POI is fused to the N-terminal domain (NTD) or C-terminal domain (CTD) or other luminal portion of TPSAN2. Advantageously this allows the POI to be luminally loaded into the EV and thus protected from degradation and/or reducing an immunogenicity associated with the POI.

In a further embodiment, the TSPAN2 is fused to at least two POIs. In a preferred embodiment one of the POIs is engineered into loop 1, loop 2 or both loops of TSPAN2 and a different POI is fused to the N-terminal domain (NTD) and/or C-terminal domain (CTD). In a preferred embodiment, the TSPAN2 is fused to one POI engineered into loop 2 and is fused to a second POI engineered onto the C-terminal domain (CTD).

In some embodiments the fusion protein further comprises: (i) a release domain capable of cleavage to release the POI; (ii) a linker or spacer; (iii) a multimerization domain; or (iv) at least one further POI. TSPAN2 has been discovered by the present inventors to be highly amenable to significant engineering, such as the addition of any of the above listed additional domains without losing expression of the TSPAN2 scaffold. This clearly renders TSPAN2 a highly effective and reliable scaffold protein for the development of a platform EV technology where the ability to reliably add and exchange of different protein domains within scaffold proteins is highly advantageous for the flexibility and modularity of the technology.

In some embodiments the EVs of the first aspect further comprise a second POI expressed on a separate construct. Optionally the second POI is present as a fusion protein with TSPAN2 or alternatively any classical exosomal polypeptide. Highly advantageously TSPAN2 has been found to not only tolerate significant engineering but also allows consistent stable co-localization with other protein constructs. This ability to allow good expression of another construct and thus enable co-localization of the two constructs on the same EV population is something that classical EV proteins have been unable to do and again enables TSPAN2 to be used with great versatility as a scaffold protein for EV platform engineering. It will also be appreciated that the ability to consistently engineer more than one construct into an EV allows functional multiplexing which is another significant benefit of the EVs of the present invention as compared to EVs utilising classical EV proteins as the scaffold protein. The present inventors have unexpectedly found that TSPAN2 is capable of being used as a highly effective EV protein scaffold upon which to engineer a POI whilst at the same time allowing localisation of a second construct on the same EV. This finding is highly unexpected because TSPAN2 is a paralog of CD9 which, alongside other classical EV proteins such as CD63 and CD81, has been shown to prevent colocalization with a second construct once it is engineered to include a POI.

In certain embodiments of the invention the POI may be: (i) a therapeutic protein, (ii) a binding protein for a therapeutic agent such as an RNA binding protein, viral binding protein, Fc-binding protein or small molecule binding protein, (iii) an endosomal escape moiety, (iv) a targeting moiety, (v) an albumin binding domain or (vi) a purification moiety. In more detail the therapeutic protein may be selected from: enzymes, receptors such as decoy receptors, membrane proteins, transporters, cytokines, antigens, neoantigens, immune effector molecules, ribonuclear proteins, nucleic acid binding proteins, antibodies, nanobodies, antibody fragments, antibody-drug conjugates, gene editing proteins such as CRISPR effector proteins including Cas proteins, TALENs, meganucleases. As can be seen from the Examples, TSPAN2 has been highly advantageously found to not only be useful in loading an extremely large range of different POIs but it has been surprisingly found to be up to 10 fold more effective than classical EV scaffold proteins. TSPAN2 has never previously been identified as a useful scaffold protein for loading POIs into exosomes let alone that it would be more effective than the currently preferred EV scaffold proteins. TSPAN2 has no known function and is therefore very little studied or understood. TSPAN2 (and TSPAN3 which was also identified as a useful scaffold protein in the Examples) show very poor interactions with CD63 or any other EV proteins which is further indication of the surprising nature of this discovery.

In another embodiment of the invention the EVs comprising TSPAN2 are further exogenously loaded with a therapeutic cargo. The exogenous loading method may be electroporation, use of a transfection reagent, co-incubation or by contact with a cell penetrating peptide (CPP) or any combination of the above. The cargo which may be exogenously loaded into the TSPAN2 expressing EV may be a protein, nucleic acid, virus, viral genome, antigen or a small molecule. Where the cargo is a nucleic acid cargo this may be: an RNA molecule, a DNA molecule or a mixmer, messenger mRNA (mRNA), antisense or splice-switching oligonucleotides, gRNA, siRNA, shRNA, miRNA, Doggybone® DNA (dbDNA®), plasmid DNA (pDNA), supercoiled or unsupercoiled plasmids, mini-circle etc. Exogenous addition of a therapeutic cargo has the added advantage that allows even greater versatility of the EV as a platform technology, i.e. an EV expressing one or more constructs which for instance may act as brain targeting moiety plus an endosomal escape moiety can then be utilized as a generic brain targeted EV into which any number of different therapeutic cargos can be added.

Another embodiment of the invention relates to a population of EVs according to the first aspect wherein the TSPAN2-POI fusion protein and the second POI construct are expressed on the same EV. Such a population of EVs reliably expressing two constructs co-localised to the same EV are advantageous for the same reasons detailed above, i.e. the inability of existing EV scaffold proteins to enable such co-localization plus the ease of utility as a platform tool, providing the ability to allow multiplexing of POIs.

In second and third aspects the present invention provides a polypeptide construct comprising TSPAN2 fused to a POI as well as a polynucleotide construct encoding such a polypeptide construct.

In a fourth aspect, the present invention also relates to a cell comprising the polypeptide construct comprising TSPAN2 fused to a POI, or polynucleotide construct encoding such a fusion protein. Optionally in further embodiments the cells may also comprise a second polypeptide construct or polynucleotide construct capable of expressing a POI. Such cells may be transiently modified to comprise such polynucleotide or polypeptide constructs or they may be stably modified to comprise at least one monocistronic, bicistronic or multicistronic polynucleotide construct. One significant advantage of using TSPAN2 as the scaffold protein is that it allows for the first time the development of cell lines, whether transiently transfected or stable cell lines, that reliably express engineered fusion protein constructs especially those where the expression of more than one construct is required. This renders TSPAN2 uniquely useful in the development of clinical grade cell lines for the production of therapeutic EVs/exosomes because it is essential that a clinical grade cell line reliably express the POI or POIs of interest over a sustained period of time. This is in fact a requirement of a clinical grade cell line due to quality control and batch release criteria to ensure consistency in the EV product. In the specific case of establishment of a stable cell line, this bypasses transfection of parental cells and thus largely eases EV production procedure. In addition, the expression level of transgene in stable producer cells is more controllable compared to transient transfection, thereby reducing the variability of EV product.

The term “stably modified” refers to cell lines which are able to pass introduced polynucleotide constructs to their progeny (i.e. daughter cells), either because the transfected DNA has been incorporated into the endogenous chromosomes or via stable inheritance of exogenous chromosomes. A stable cell line may be defined as a homogenous cell population that retains 70% or more, 80% or more, or even 90% or more of volumetric productivity titer over at least 50 generations, optionally over 60 generations, over 70 generations, over 85 generations or even over 100 generations. It may demonstrate “no clinically meaningful differences” over at least 50 generations, optionally over 60 generations, over 70 generations, over 85 generations or even over 100 generations, as determined by structure, function, purity, chemical identity and/or bioactivity. Ideally, the cells may show less than 15% deviation, less than 10% deviation or even less than 5% deviation (within the limits of measurement) in production titres at the end of 100 doubling cycles.

As demonstrated in the Examples, the present inventors have also identified TSPAN3 and TSPAN18 as improved EV scaffold proteins. The present invention therefore also relates to an EV comprising a fusion protein, wherein the fusion protein comprises TSPAN3 or TSPAN18 fused to a POI. Optionally the POI may be engineered into loop1, loop2 or both loops of TSPAN3 or TSPAN18 to facilitate surface display of the POI. Alternatively the POI may be engineered into the NTD and/or CTD or other luminal portion of TSPAN3 or TSPAN18 to facilitate luminal loading of the POI. In other embodiments the TSPAN3 or TSPAN18 fusion protein further comprises: a release domain capable of cleavage to release the POI; a linker or spacer; a multimerization domain; and/or at least one further POI. In certain embodiments the TSPAN3 or TSPAN18-containing EV further comprises a second POI expressed on a separate construct, optionally wherein the second POI is present as a fusion protein with an exosomal polypeptide. Similar to the situation described above for TSPAN2, the TSPAN3 or TSPAN18 EVs can be exogenously loaded with a therapeutic cargo (such as an nucleic acid, viral or small molecule cargo) by any known mechanism of exogenous loading including: electroporation, transfection reagent, co-incubation or by contact with a CPP or any combination of these methods. The present invention also relates to pharmaceutical compositions comprising the TSPAN3-POI or TSPAN18-POI EVs. Furthermore, the present invention relates to a method of treatment comprising administering to a patient in need thereof, an effective amount of TSPAN3-POI or TSPAN18-POI EVs and similarly the use of the TSPAN3-POI or TSPAN18-POI EVs for use in medicine.

The present invention also relates to a polypeptide construct comprising TSPAN3 or TSPAN19 fused to a POI or a polynucleotide construct encoding such polypeptide constructs. The present invention future relates to cell comprising such TSPAN3 or TSPAN18 polypeptide or polynucleotide constructs, optionally said cell comprising a second polypeptide construct or polynucleotide construct capable of expressing a second POI.

Naturally the present invention also relates to a method for producing EVs, said method comprising: (i) introducing into an EV-producing cell a polynucleotide construct encoding a TSPAN3/TSPAN18-POI fusion construct; and (ii) expressing the construct in the EV-producing cell, thereby generating EVs comprising the TSPAN3/18-POI fusion protein. The method described may also comprise an intermediary step of introducing into the same EV-producing cell a second polynucleotide construct encoding a second POI (optionally wherein the second POI is present in the form of a fusion protein with an EV protein); such that both constructs are expressed in the EV-producing cell, thereby generating EVs comprising the TSPAN3/18-POI fusion protein and the second POI.

### Definitions

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the TSPAN2 fusion proteins described herein in are to be understood to be disclosed, relevant, and compatible with all other aspects, teachings and embodiments herein, for instance aspects and/or embodiments relating to the methods for producing or purifying the EVs, or relating to the corresponding polynucleotide constructs described herein or the engineered EV-producing cells from which the EVs derive. Furthermore, certain embodiments described in connection with certain aspects, for instance the administration routes of the EVs comprising the therapeutic cargo molecule and optionally the fusion polypeptides, as described in relation to aspects pertaining to treating certain medical indications, may naturally also be relevant in connection with other aspects and/or embodiment such as those pertaining to the pharmaceutical compositions comprising such EVs. Furthermore, all polypeptides and proteins identified herein can be freely combined in fusion proteins using conventional strategies for fusing polypeptides. As a non-limiting example, a TSPAN2-POI fusion protein described herein may be freely combined in any combination with one or more targeting moiety, endosomal escape moiety, albumin binding domain, additional POI, optionally combined with all other polypeptide domains, regions, sequences, peptides, groups herein, e.g. any multimerization domains, linker sequences, release domains, etc. Furthermore, when teachings herein refer to EVs in singular and/or to EVs as discrete natural nanoparticle-like vesicles it should be understood that all such teachings are equally relevant for and applicable to a plurality of EVs and populations of EVs. As a general remark, the therapeutic POI, binding protein for a therapeutic agent, endosomal escape moiety, albumin binding domain or purification domain and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the desired technical effect associated therewith. As long as their biological properties are maintained the polypeptide and/or polynucleotide sequences according to the present application may deviate with as much as 50% (calculated using, for instance, BLAST or ClustalW) as compared to the native sequence, although a sequence identity or similarity that is as high as possible is preferable (for instance 60%, 70%, 80%, or e.g. 90% or higher). Standard methods in the art may be used to determine homology. For example, PILEUP and BLAST algorithms can be used to calculate homology or line up sequences. The combination (fusion) of e.g. several polypeptides implies that certain segments of the respective polypeptides may be replaced and/or modified and/or that the sequences may be interrupted by insertion of other amino acid stretches, meaning that the deviation from the native sequence may be considerable as long as the key properties (e.g. retaining its therapeutic effect, or ability to bind to a therapeutic cargo or to bind to albumin and therefore extend half-life, ability to traffic a fusion construct to an EV, targeting capabilities, etc.) are conserved. Similar reasoning thus naturally applies to the polynucleotide sequences encoding for such polypeptides. Any accession numbers or SEQ ID NOS mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein but also variants and derivatives thereof. All accession numbers referred to herein are UniProtKB accession numbers, and all proteins, polypeptides, peptides, nucleotides and polynucleotides mentioned herein are to be construed according to their conventional meaning as understood by a skilled person.

### EVs:

The terms “extracellular vesicle” or “EV” are used interchangeably herein and can be understood to relate to any type of vesicle that is obtainable from a cell in any form. The size of EVs may vary considerably, but an EV typically comprises a volume defined by a bi-lipid membrane and having a nano-sized hydrodynamic radius, i.e., a radius below 1000 nm. The volume may comprise the vesicular secretome. The different types of EVs are defined by different morphologies, structure, messages and function. EVs can be broadly divided into two categories, (1) ectosomes and (2) exosomes. Some examples of EVs include for instance an exosome, an apoptotic body, arrestin domain containing protein 1 [ARRDC1]-mediated microvesicles (ARMMs), an ectosome, such as a microparticle or microvesicle, or a cardiosome, etc. Essentially, the terms ‘extracellular vesicle’ and/or ‘EV’ may relate to any type of lipid-based structure (with vesicular morphology or with any other type of suitable morphology) that can act as a delivery or transport vehicle or that has native therapeutic or pharmacological effects.

Furthermore, the said terms can be understood to also relate to, in some embodiments, extracellular vesicle mimics, cellular membrane vesicles obtained through membrane extrusion, sonication or other techniques, etc.

Clearly, EVs may be derived from any cell type, whether in vivo, ex vivo or in vitro (further details of suitable source or producer cells are herein described below).

Exosomes, microvesicles and ARMMs are just some examples of the different subtypes that fall under the hereinbefore broader description of EVs and represent particularly preferable EVs, but it will be appreciated that other EVs may also be advantageous in certain circumstances. Advantageously, the EV is an exosome.

The terms “apoptotic body” or “apoptotic bodies” are used interchangeably herein and can be understood to relate to any type of vesicle that is obtainable or derivable from apoptotic cells. Typically, an apoptotic body has a diameter ranging from about 1 μm to about 5 μm.

The terms “cardiosome” or “cardiosomes” are used interchangeably herein and can be understood to relate to any type of vesicle that is obtainable or derivable from cardiac cells.

The terms “ectosome” or “ectosomes” are used interchangeably herein and can be understood to relate to any type of heterogenous vesicle that is obtainable or derivable from outward budding of the plasma membrane and/or cell membrane of a cell, preferably from neutrophils and monocytes in serum. Examples of ectosomes include, but are not limited to, microvesicles, microparticles and large vesicles. Typically, ectosomes range in size from about 50 nm to about 1 μm.

The terms “microparticle” or “microparticles” are used interchangeably herein and can be understood to relate to any type of vesicle that is obtainable or derivable from platelets.

The terms “microvesicle”, and “microvesicles” are used interchangeably herein and can be understood to relate to any type of vesicle that is obtainable or derivable or shed from the plasma membrane or cell membrane of a cell.

The term “ARMMs” can be understood to relate to any type of vesicle that is obtainable or derivable from the plasma membrane or cell membrane of a cell, from which they bud directly. Such microvesicles are mediated by the ARRDC1 and typically lack known late endosomal markers. As such, ARMMs are distinct from exosomes herein described.

The terms “exosome” or “exosomes” are used interchangeably herein and can be understood to relate to any type of vesicle that is obtainable or derivable from the endosomal, lysosomal and/or endo-lysosomal pathway and/or from inward budding of the plasma membrane and/or cell membrane. Exosomes often have a size of from about 30 to about 300 nm, typically in the range of from about 40 to about 250 nm, and sometimes from about 40 to about 160 nm, which is a highly suitable size range.

The term “modified” can mean that the vesicle has been modified either using genetic or chemical approaches, for instance via genetic engineering of the EV-producing cell, preferably an exosome-producing cell or via e.g., chemical conjugation, for instance to attach moieties to the EV, preferably the exosome surface.

The terms “genetically modified” and “genetically engineered” are used interchangeably herein and can mean that the EV, preferably an exosome, is derived from a genetically modified/engineered cell or is otherwise genetically engineered to express and/or modify the expression of proteins in the lumen, extravesicular membrane and/or displayed on the surface of the EV (e.g., exosome), which is typically incorporated into the EVs, preferably exosomes, produced by those cells. Such genetically engineered or genetically modified EVs do not occur in nature.

Furthermore, the said terms shall be understood to also relate to, in some embodiments, EV mimics, cellular membrane vesicles obtained through membrane extrusion, sonication or other techniques, etc.

It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml or per litre), or any other number larger, smaller or anywhere in between. In the same vein, the term “population”, which may e.g. relate to an EV comprising a certain POI shall be understood to encompass a plurality of entities which together constitute such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the albumin binding domain (ABD), therapeutic cargo, any targeting moieties present on the EVs, the pharmaceutical formulation, etc.

### TSPAN2:

TSPAN2 shall be understood to mean tetraspanin-2, otherwise referred to as TSN2, or Tetraspan NET-3, as well as derivatives, domains, variants, mutants, or regions thereof. (Database references for TSPAN2 include: HGNC: 20659, NCBI Entrez Gene: 10100, Ensembl: ENSG00000134198, OMIM®: 613133, UniProtKB/Swiss-Prot: 060636). The TSPAN2 protein is a multi-pass transmembrane protein with 4 transmembrane regions. TSPAN 2 is a paralogue of CD9 and is believed to play a role in signalling in oligodendrocytes in the early stages of their terminal differentiation into myelin-forming glia and may also function in stabilizing the mature sheath. The sequence of TSPAN2 is provided in SEQ ID Nos: 1 and 2. Derivatives, domains, variants, mutants or regions of TSPAN2, envisaged as forming part of the invention, can be a protein having at least 10% sequence identity with SEQ ID No: 1 and/or 2. For example, the derivative, domain, variant, mutant or region may have at least 20%, or even at least 30%, sequence identity with SEQ ID No: 1 and/or 2. The derivative, domain, variant, mutant or region may have “substantially the nucleotide and/or amino acid sequence” of SEQ ID No: 1 and/or 2, which can mean a sequence that has at least 40% sequence identity with the sequence of SEQ ID No: 1 and/or 2. Accordingly, in an embodiment, derivative, domain, variant, mutant or region has at least 50%, at least 60%, at least 70%, at least 80%, at least 90% or at least 95% sequence identity with SEQ ID No: 1 and/or 2. Loop 1 of TSPAN2 is defined by residues 35-54 and Loop 2 of TSPAN2 is defined by residues 112-188.

### TSPAN3:

TSPAN3 shall be understood to mean Tetraspanin 3, otherwise referred to as TSN3, Transmembrane 4 Superfamily Member 8, TSPAN-3, TM4SF8, TM4-A, Tetraspanin TM4-A, Tetraspanin-3, as well as derivatives, domains, variants, mutants, or regions thereof. (Database references for TSPAN3 include: HGNC: 17752, NCBI Entrez Gene: 10099, Ensembl: ENSG00000140391, OMIM®: 613134, UniProtKB/Swiss-Prot: 060637). TSPAN3 is member of the transmembrane 4 superfamily, also known as the tetraspanin family. TSPAN3 is a paralog of CD82 and is believed to regulate the proliferation and migration of oligodendrocytes, a process essential for normal myelination and repair. The sequence of TSPAN3 is given by SEQ ID Nos: 3 and 4. Derivatives, domains, variants, mutants or regions of TSPAN3, envisaged as forming part of the invention, can be a protein having at least 10% sequence identity with SEQ ID No: 3 and/or 4. For example, the derivative, domain, variant, mutant or region may have at least 20%, or even at least 30%, sequence identity with SEQ ID No: 3 and/or 4. The derivative, domain, variant, mutant or region may have “substantially the nucleotide and/or amino acid sequence” of SEQ ID No: 3 and/or 4, which can mean a sequence that has at least 40% sequence identity with the sequence of SEQ ID No: 3 and/or 4. Accordingly, in an embodiment, derivative, domain, variant, mutant or region has at least 50%, at least 60%, at least 70%, at least 80%, at least 90% or at least 95% sequence identity with SEQ ID No: 3 and/or 4.

### TSPAN18:

TSPAN18 shall be understood to mean Tetraspanin-18, as well as derivatives, domains, variants, mutants, or regions thereof. (Database references to TSPAN18 include: HGNC: 20660, NCBI Entrez Gene: 90139, Ensembl: ENSG00000157570, OMIM®: 619399, UniProtKB/Swiss-Prot: Q96SJ8.) Little is known of the function of TSPAN18 but it is predicted to be integral component of plasma membrane and is a paralogue of TSPAN1. The sequence of TSPAN18 is given by SEQ ID Nos: 5 and 6. Derivatives, domains, variants, mutants or regions of TSPAN18, envisaged as forming part of the invention, can be a protein having at least 10% sequence identity with SEQ ID No: 5 and/or 6. For example, the derivative, domain, variant, mutant or region may have at least 20%, or even at least 30%, sequence identity with SEQ ID No: 5 and/or 6. The derivative, domain, variant, mutant or region may have “substantially the nucleotide and/or amino acid sequence” of SEQ ID No: 5 and/or 6, which can mean a sequence that has at least 40% sequence identity with the sequence of SEQ ID No: 5 and/or 6. Accordingly, in an embodiment, derivative, domain, variant, mutant or region has at least 50%, at least 60%, at least 70%, at least 80%, at least 90% or at least 95% sequence identity with SEQ ID No: 5 and/or 6.

### POI:

The POI may be any protein or polypeptide that is desired to be loaded into or onto an EV. Exemplary POIs may be i) therapeutic proteins, (ii) binding proteins for a therapeutic agent such as an RNA binding protein, viral binding protein, small molecule binding proteins, fc-binding proteins (iii) an endosomal escape moiety, (iv) a targeting moiety, (v) an albumin binding domain, or (vi) a purification moiety. In the detailed embodiments described below any of the specific POIs may be comprised in the TSPAN2 fusion polypeptide. Alternatively the POI may instead or as well be comprised in the EV as a second POI expressed on a separate construct.

As previously mentioned, the advantage of using TSPAN2 as the scaffold protein is that it allows the development of cell lines which have undergone significant engineering whilst retaining strong consistent expression of either single or multiple protein constructs. As stated above this is particularly advantageous when engineering into the loops/NTD/CTD of TSPAN2 and also very advantageous when expressing more than one construct in the same EV, as TSPAN2 allows co-localization of constructs. Given below are some non-limiting exemplary advantageous constructs and pairings of constructs, which may be engineered into EVs with greater certainty and stability when using TSPAN2:

### Single POI Fused to TSPAN2

TSPAN2-surface displayed therapeutic protein
TSPAN2-lumenally loaded therapeutic protein
TSPAN2-binding protein for therapeutic agent
TSPAN2-surface displayed targeting moiety
TSPAN2-surface displayed ABD moiety
TSPAN2-endosomal escape moiety

Additionally, any of the fusion constructs listed above may be expressed in the EVs/cells of the present invention in combination with a second construct comprising any POI such that the EV comprises at least two different protein constructs. Optionally the second construct is a fusion protein of an EV protein-POI.

### Double POI Fused to TSPAN2

TSPAN2-therapeutic protein in both Loop1 and Loop2
TSPAN2-therapeutic protein in Loop1/2 and therapeutic protein on NTD/CTD
TSPAN2-therapeutic protein in Loop1/2 and targeting moiety in Loop1/2
TSPAN2-therapeutic protein in Loop1/2 and endosomal escape moiety in Loop1/2
TSPAN2-therapeutic protein in Loop1/2 and ABD in Loop1/2
TSPAN2-therapeutic protein on both NTD and CTD
TSPAN2-therapeutic protein on NTD/CTD (luminal) and targeting moiety in Loop1/2
TSPAN2-therapeutic protein on NTD/CTD (luminal) and ABD in Loop1/2
TSPAN2-therapeutic protein on NTD/CTD (luminal) and endosomal escape moiety in Loop1/2
TSPAN2-binding protein in both Loop1 and Loop2
TSPAN2-binding protein in Loop1/2 and targeting moiety in Loop1/2
TSPAN2-binding protein in Loop1/2 and endosomal escape moiety in Loop1/2
TSPAN2-binding protein in Loop1/2 and ABD in Loop1/2
TSPAN2-binding protein on both NTD and CTD

TSPAN2-binding protein on NTD/CTD (luminal) and targeting moiety in Loop1/2
TSPAN2-binding protein on NTD/CTD (luminal) and ABD in Loop1/2
TSPAN2-binding protein on NTD/CTD (luminal) and endosomal escape moiety in Loop1/2
TSPAN2-targeting moiety in Loop1/2 and endosomal escape moiety in Loop1/2
TSPAN2-targeting moiety in Loop1/2 and ABD in Loop1/2
TSPAN2-targeting moiety in Loop1/2 and purification moiety in Loop1/2
TSPAN2-endosomal escape moiety in Loop1/2 and ABD in Loop1/2
TSPAN2-endosomal escape moiety in Loop1/2 and purification moiety in Loop1/2
TSPAN2-purification moiety in Loop1/2 and ABD in Loop1/2

Additionally, any of the fusion constructs listed above may be expressed in the EVs/cells of the present invention in combination with a second construct comprising any POI such that the EV comprises at least two different protein constructs. Optionally the second construct is a fusion protein of an EV protein-POI.

Particularly preferred combinations include a therapeutic protein or a binding protein for a therapeutic agent fused to TSPAN2 plus a second construct comprising the endosomal escape moiety VSVG, optionally wherein the VSVG is fused to TSPAN2 or a classical EV protein.

In other embodiments the TSPAN2 feature of the above listed constructs may be replaced with either TSPAN3 or TSPAN18 with similar effect. The present invention includes polynucleotides encoding for any of the polypeptide fusion constructs listed above whether comprising TSPAN3 or TSPAN18 in place of TSPAN2.

In certain embodiments the POI, or at least one of the POIs, may be a therapeutic protein. In one embodiment the therapeutic POI is comprised in the TSPAN2 fusion polypeptide. Alternatively the POI that is fused to TSPAN2 may be any of the other exemplary POIs and the therapeutic POI may instead be expressed on a separate construct.

The therapeutic protein may be selected from: enzymes, receptors such as decoy receptors, membrane proteins, transporters, cytokines, antigens, neoantigens, immune effector molecules, ribonuclear proteins, nucleic acid binding proteins, antibodies, nanobodies, antibody fragments, antibody-drug conjugates, gene editing proteins such as CRISPR effector proteins includingCas proteins, transcription activator-like effector nucleases (TALENs), meganucleases.

In more detail the therapeutic protein cargos (POIs) according to the present invention include: antibodies, intrabodies, nanobodies, single chain variable fragments (scFv), affibodies, bi- and multispecific antibodies or binders including bispecific T-cell engagers (BiTEs), receptors, ligands, transporters, enzymes for e.g. ERT or gene editing, tumour suppressors, viral or bacterial inhibitors, cell component proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins such as cytokines and inhibitors of such cytokines (cytokines may include: CXCL8, GMCSF, interleukins including: IL-1 family, IL-2, IL-4, IL-6, IL-6-like, IL-9, IL-10, IL12, IL-13, IL-17, Interferons including INF-alpha/beta/gamma, TNF family members, CD40 and CD40L, TRAIL, and TGF-beta family) mitochondrial proteins, and heat shock proteins, etc. The cargo protein may be a reporter protein such as green fluorescent protein (GFP) or nanoLuc.

In one preferred embodiment, the encoded protein is a CRISPR-associated (Cas) polypeptide (such as Cas9) with intact nuclease activity which is associated with (i.e. carries with it) an RNA strand that enables the Cas polypeptide to carry out its nuclease activity in a target cell once delivered by the peptide. The CRISPR (Clustered Regularly Interspaced Short Palindromic Repeats) components according to the present invention include CRISPR components that are derived from any bacterial source. The CRISPR components may come from class 1 or class 2, specifically the Cas type may be Cas type I, II, III, IV, V or VI. The specific Cas protein may be Cas9, Cas12 (Cas12a or Cas12b), C2c2, Cpf1, Cas10, Cas13 (cas13a, Cas13b or Cas13c), Cas3, a Cas14 protein, a CasX protein, or a CasY protein, CasMINI, or SuperFi-Cas9. The CRISPR protein may be a CRISPR nuclease, a CRISPR nickase, or a nuclease deficient CRISPR variant. Alternatively, in another preferred embodiment, the Cas polypeptide may be catalytically inactive, to enable targeted genetic engineering. Yet another alternative may be any other type of CRISPR effector such as the single RNA guided endonuclease Cpf1. The inclusion of Cpf1 is a particularly preferred embodiment of the present invention, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression.

Additional preferred embodiments include therapeutic protein cargos selected from the group comprising enzymes or transporters for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase.

Additional preferred embodiments include therapeutic protein cargos selected from the group comprising enzymes associated with Urea cycle disorders including: N-Acetylglutamate synthase, carbamoyl phosphate synthetase, ornithine transcarbamoylase, argininosuccinic acid synthase, argininosuccinic acid lyase, arginase, mitochondrial ornithine transporter, citrin, y+L amino acid transporter 1, uridine monophosphate synthase UMPS.

In other preferred embodiments, the Pol may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The Pols are typically proteins or peptides of human origin unless indicated otherwise by their name, any other nomenclature, or as known to a person skilled in the art, and they can be found in various publicly available databases such as Uniprot, RCSB, etc.

In another preferred embodiment the therapeutic cargo is an antigen/neoantigen, optionally wherein the antigen/neoantigen is suitable for use in cancer immunotherapy.

Any antigen/neoantigen may be incorporated into the EVs of the present invention. The antigens may be suitable for raising immune responses against pathogens such as bacteria, viruses, funguses or the antigen may be a tumor antigen useful in eliciting an immune response against a tumor for cancer immunotherapy. There may be one or more antigens/neoantigens present in any EV according to the invention. The one or more antigens/neoantigens may be endogenous/autologous (coming from the subject itself) or exogenous/allogenic (coming from another subject) or in the case of more antigens/neoantigens being incorporated into/onto the EVs the antigens/neoantigens may be any mix of autologous/allogenic antigens. Preferably the antigens are autologous. Moreover, the one or more antigens/neoantigens may have any origin such as e.g. viral or bacterial or may be a tumour antigen and furthermore may be immunostimulatory or immunosuppressive or a combination thereof. The antigen/neoantigen maybe be useful in the treatment of any disease by immunotherapy. The treatment of cancer by immunotherapy is a particularly preferred embodiment. Where the antigen is a neo-antigen it may be identified by sequencing of a tumour to identify the neo-antigen.

Exemplary tumour antigens may be: Alphafetoprotein (AFP), Carcinoembryonic antigen (CEA), CA-125, MUC-1, Epithelial tumor antigen (ETA), Melanoma-associated antigen (MAGE), WT-1, NY-ESO-1, LY6K, IMP3, DEPDC1, CDCA-1, abnormal products of ras, p53, KRAS, or NRAS, CTAG1B, Peptides derived from chromosomal translocations such as BCR-ABL or ETV6-AML1, viral antigens such as peptides from HPV-related cancers, peptides derived from proteins such as tyrosinase, gp100/pmel17, Melan-A/MART-1, gp75/TRP1, or TRP2, and overexpressed antigens such as MOK (RAGE-1), ERBB2 (HER2/NEU).

Where the therapeutic cargo is an antigen or neoantigen the EV or pharmaceutical composition comprising the EV may optionally further comprise an adjuvant. Where the antigen is administered with an adjuvant to stimulate the immune response the adjuvant may be: an Inorganic compound: such as aluminium hydroxide, aluminium phosphate, calcium phosphate hydroxide, a mineral oil such as paraffin oil, bacterial products such as killed bacteria Bordetella pertussis, Mycobacterium bovis, toxoids, a nonbacterial organic such as squalene, a detergent such as Quil A, a plant saponin, a cytokine such as IL-1, IL-2, IL-12, or RIBI (muramyl dipeptides) or ISCOM (immunostimulating complexes) such as STING (stimulator of interferon genes) agonists which can include cyclic dinucleotides. Such adjuvants may protect the therapeutic EV from rapid dispersal by sequestering it in a local deposit, or they may contain substances that stimulate the host to secrete factors that are chemotactic for macrophages and other components of the immune system. Adjuvants that can be incorporated to a vaccine are well-known by the person skilled in the art and will be selected, in such a way that they do not negatively affect the immunological activity of the EV. Where the adjuvant is a protein (for example a cytokine) in specific embodiments the adjuvant may be incorporated into the EV itself as part of the TSPAN2 fusion protein or the second POI.

In certain embodiments, the POI or at least one of the POIs may be a binding protein for a therapeutic agent. Such binding proteins may be for instance RNA or DNA binding proteins, viral binding proteins, small molecule binding proteins or Fc-binding proteins. In some embodiments the binding protein capable of binding to the therapeutic agent may be the POI comprised in the TSPAN2 fusion polypeptide. Alternatively the binding protein may be present as a second POI expressed on a separate construct.

In an embodiment, the POI is a nucleic acid binding protein (NA-binding protein) such as an RNA or DNA binding protein. In these embodiments nucleic acid cargos which are loaded into the EVs by the binding of the nucleic acid to the NA-binding protein.

Non-limiting examples of NA-binding proteins are Ago2, Dicer, Drosha, DGCR8, hnRNPA1, hnRNPA2B1, DDX4, ADAD1, DAZL, ELAVL4, IGF2BP3, SAMD4A, TDP43, FUS, FMR1, FXR1, FXR2, EIF4A13, the MS2 coat protein, as well as any domains, parts or derivates, thereof. More broadly, particular subclasses of RNA-binding proteins and domains, e.g. mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins and transcription factors (TFs). Furthermore, various domains and derivatives may also be used as the NA-binding domain to transport an NA cargo into EVs. Non-limiting examples of RNA-binding domains include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae. Such RNA-binding domains may be present in a plurality, alone or in combination with others, and may also form part of a larger RNA-binding protein construct as such, as long as their key function (i.e. the ability to transport an NA cargo of interest, e.g. an mRNA or a short RNA) is maintained.

In preferred embodiments the present invention relates to two groups of NA-binding domains, namely PUF proteins and CRISPR-associated polypeptides (Cas), specifically Cas9, Cas6 and Cas13, as well as various types of NA-binding aptamers. The present invention uses the term PUF proteins to encompass all related proteins and domains of such proteins (which may also be termed PUM proteins), for instance human Pumilio homolog 1 (PUM1), PUMx2 or PUFx2 which are duplicates of PUM1, etc., or any NA-binding domains obtainable from any PUF (PUM) proteins. PUF proteins are typically characterized by the presence of eight consecutive PUF repeats, each of approximately 40 amino acids, often flanked by two related sequences, Csp1 and Csp2. Each repeat has a ‘core consensus’ containing aromatic and basic residues. The entire cluster of PUF repeats is required for RNA binding. The PUF proteins as per the present invention can be natural or engineered to bind anywhere in an RNA molecule, or alternatively one can choose PUF proteins with different binding affinities for different sequences and engineer the RNA molecule to contain said sequence. There is furthermore engineered and/or duplicated PUF domains that bind 16-nucleotides in a sequence-specific manner, which can also be utilized to increase the specificity for the NA cargo molecule further. Hence the PUF domain can be modified to bind any sequence, with different affinity and sequence length, which make the system highly modular and adaptable for any RNA cargo molecule as per the present invention. PUF proteins and regions and derivatives thereof that may be used as NA-binding domains as per the present invention include the following non-limiting list of PUF proteins: FBF, FBF/PUF-8/PUF-6,-7,-10, all from C. elegans; Pumilio from D. melanogaster; Puf5p/Mpt5p/Uth4p, Puf4p/Ygl014wp/Ygl023p, Puf5p/Mpt5p/Uth4p, Puf5p/Mpt5p/Uth4p, Puf3p, all from S. cerevisiae; PufA from Dictyostelium; human PUM1 (Pumilio 1, sometimes known also as PUF-8R) and any domains thereof, polypeptides comprising NA-binding domains from at least two PUM1, any truncated or modified or engineered PUF proteins, such as for instance PUF-6R, PUF-9R, PUF-10R, PUF-12R, and PUF-16R or derivatives thereof; and X-Puf1 from Xenopus. Particularly suitable NA-binding PUFs as per the present invention includes the following: PUF 531, PUF mRNA loc (sometimes termed PUFengineered or PUFeng), and/or PUFx2, (sequences of which are available in PCT/EP2018/080681) and any derivatives, domains, and/or regions thereof. The PUF/PUM proteins are highly advantageous as they may be selected to be of human origin. Furthermore, as is the case with the PUF proteins, Cas proteins such as Cas 6, Cas 9 and Cas13 are highly preferred examples of releasable NA-binding domains which bind with suitable affinity to NA cargo molecules, thereby enabling a releasable, reversible attachment of the Cas protein to the NA cargo. As with the PUF-based NA-binding domains, the Cas proteins represent a releasable, irreversible NA-binding domain with programmable, modifiable sequence specificity for the target NA cargo molecule, enabling higher specificity at a lower total affinity, thereby allowing for both loading of the NA cargo into EVs and release of the NA cargo in a target location.

A “nucleic acid” refers to a polynucleotide and includes polyribonucleotides and poly-deoxyribonucleotides. Nucleic acids according to the present invention may include any polymer or oligomer of pyrimidine and purine bases, e.g., cytosine, thymine, and uracil, and adenine and guanine, respectively. (See Albert L. Lehninger, Principles of Biochemistry, at 793-800 (Worth Pub. 1982) and G. Michael Blackburn, Michael J. Gait, David Loakes and David M. Williams, Nucleic Acids in Chemistry and Biology 3rd edition, (RSC publishing 2006) which are herein incorporated in their entirety for all purposes). Indeed, the present invention contemplates any deoxyribonucleotide or ribonucleotide component, and any chemical variants thereof. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present invention may be a peptide nucleic acid (PNA; see U.S. Pat. No. 6,156,501, which is hereby incorporated by reference in its entirety.) The invention also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes, for instance, cDNA, RNA, DNA/RNA hybrid, antisense RNA, siRNA, mRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The present invention specifically relates to ABD-EVs which are further loaded with nucleic acids such as siRNAs which target oncogenes known to be involved with the development of cancer. The genes targeted by the nucleic acids according to the present invention may be ABL, AF4/HRX, AKT-2, ALK, ALK/NPM, AML1, AML1/MTG8, AXL, BCL-2, 3, 6, BCR/ABL, c-MYC, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HER2/neu, HOX11, HST, IL-3, INT-2, JUN, KIT, KS3, K-SAM, LBC, LCK, LMO1, LMO2, L-MYC, LYL-1, LYT-10, LYT-10/Ca1, MAS, MDM-2, MLL, MOS, MTG8/AML1, MYB, MYH11/CBFB, NEU, N-MYC, OST, PAX-5, PBX1/E2A, PIM-1, PRAD-1, RAF, RAR/PML, RAS-H, RAS-K, RAS-N, REL/NRG, RET, RHOM1, RHOM2, ROS, SKI, SIS, SET/CAN, SRC, TAL1, TAL2, TAN-1, TIAM1, TSC2, TRK.

In more detail the nucleic acid cargo molecule which is bound to the RNA/DNA binding protein (POI) of the invention or exogenously loaded (described further herein) into the exosome may be selected from the group comprising shRNA, siRNA, saRNA, miRNA, an anti-miRNA, mRNA, modified mRNA, gRNA, pri-miRNA, pre-miRNA, circular RNA, piRNA, tRNA, rRNA, snRNA, lncRNA, ribozymes, mini-circle DNA, plasmid DNA, RNA/DNA vectors, trans-splicing oligonucleotides, splice-switching oligonucleotides, CRISPR guide strands, morpholinos (PMO) antisense oligonucleotides (ASO), peptide-nucleic acids (PNA), a viral genome and viral genetic material (for instance a naked AAV genome), but essentially any type of nucleic acid molecule can be delivered by the EVs of the present invention. Both single-stranded and double-stranded nucleic acid molecules are within the scope of the present invention, and the nucleic acid molecule may be naturally occurring (such as RNA or DNA) or may be a chemically synthesised RNA and/or DNA molecule which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

When the cargo is an mRNA the mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine. A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component. A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein. An mRNA may include a 5′ untranslated region, a 3′ untranslated region, and/or a coding or translating sequence, which is translated to create the fusion protein of the present invention. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In some embodiments, an mRNA may include a 5′ cap structure, a chain terminating nucleotide, a stem loop, a polyA sequence, and/or a polyadenylation signal. A cap structure or cap species is a compound including two nucleoside moieties joined by a linker which caps the mRNA at its 5′ end, and which may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G (5′) ppp (5′) G, commonly written as m7GpppG. A cap species may also be an anti-reverse cap analog. A non-limiting list of possible cap species includes m7GpppG, m7Gpppm7G, m73′dGpppG, iri27′03′GpppG, iri27′03′GppppG, iri27′02′GppppG, m7Gpppm7G, m73′dGpppG, iri27′03′GpppG, iri27′03′GppppG, and m27 02′GppppG. An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. An mRNA may instead or additionally include a stem loop, such as a histone stem loop. A stem loop may include 1, 2, 3, 4, 5, 6, 7, 8, 9 or more nucleotide base pairs. For example, a stem loop may include 4, 5, 6, 7, 8, 9 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. The modified mRNA of the present invention may comprise in addition to the coding region (which codes for the fusion protein and which may be codon-optimized) one or more of a stem loop, a chain terminating nucleoside, miRNA binding sites, a polyA sequence, a polyadenylation signal, 3′ and/or 5′ untranslated regions (3′ UTRs and/or 5′ UTRs) and/or a 5′ cap structure. As abovementioned, various nucleotide modifications are preferably incorporated into the mRNA to modify it for increased translation, reduced immunogenicity, and increased stability. Suitable modified nucleotides include but are not limited to N1-methyladenosine (m1A), N6-methyladenosine (m6A), 5-methylcytidine (m5C), 5-methyluridine (m5U), 2-thiouridine (s2U), 5-methoxyuridine (5moU), pseudouridine (ψ), N1-methylpseudouridine (m1ψ). Among these mRNA modifications, m5C and ψ are the most preferred as they reduce the immunogenicity of mRNA as well as increase the translation efficiency in vivo. In preferred embodiments of the present invention, the composition herein comprises a modified mRNA as the polynucleotide cargo, wherein the mRNA is modified with at least 50% m5C and 50% ψ or m1ψ, preferably at least 75% m5C and 75% ψ or m1ψ, and even more preferably 90% m5C and 90% w or m1ψ, or even more preferably 100% modification using m5C and w or m1ψ.

In some embodiments the POI may be a viral binding protein, such as: proteins capable of binding to adeno-associated virus (AAV), for instance the AAV-receptor, or proteins capable of binding to other types of viruses such as lenti-viruses. Generally any protein capable of binding to a viral coat/envelope protein or capable of binding to a viral genome is within the scope of the invention. Exemplary viral binding proteins include: AAVR GPR108, syndecans and albumin.

In some embodiments the POI may be a small molecule binding protein, such as any protein, polypeptide, or peptide (i.e. any molecule comprising a sequence of amino acids) to which a small molecule agent can be attached, via non-covalent or covalent attachment, or via a combination of both covalent and non-covalent interactions. The combination of a binding protein and a small molecule agent is herein described using terms such as “binding protein-small molecule conjugate” or “binding protein-small molecule drug conjugate” or “binding protein-small molecule agent conjugate”, or just “conjugate”. The binding protein may play several different roles: it may for instance be (i) a carrier and/or delivery modality which is primarily meant to transport a small molecule agent attached to it, (ii) a targeting agent to direct trafficking of the EV carrying the binder protein-small molecule conjugate to a particular location, (iii) a therapeutically active protein which becomes therapeutically active or inactive through the attachment of a small molecule, which may have agonistic or antagonistic effects, (iv) a signaling protein which together with or without its small molecule cargo may exert or contribute to a cellular and/or bodily change and a related therapeutic and/or prophylactic effect, (v) a protein carrying out or catalyzing a particular reaction only when brought into the proximity of another protein, etc. The binding protein may further contribute to a bodily and/or cellular action or activity and a related therapeutic effect by releasing the small molecule agent in a suitable location, or it may contribute to such effects by retaining the small molecule agent bound to it. An example of the first case is when the binding protein releases the small molecule drug inside a target cell after EV-mediated delivery, whereas an example of the second case is the delivery of an antibody-small molecule drug conjugate into a tumor.

The term “small molecule agent” or “small molecule” or “small molecule drug” or “small molecule therapeutic” are used interchangeably herein and shall be understood to relate to any molecular agent which may be used for the treatment and/or diagnosis of a disease and/or disorder, and also for modulating or changing e.g. the activity and/or the binding and/or the location of a binding protein. Small molecule agents are normally synthesized via chemical synthesis means, but may also be naturally derived, for instance via purification from natural sources, or may be obtained through any other suitable means or combination of techniques. A brief, non-limiting definition of a “small molecule” is any organic compound with a molecular weight of less than 900 g/mol (Dalton) that may in essentially any way regulate, impact, or influence a biological process. For the purposes of this invention, small molecules may be substantially larger than 900 g/mol, for instance 1500 g/mol, 3000 g/mol, or occasionally even larger. Overall, molecular weight and/or molecular size is not a defining factor behind what constitutes a small molecule agent. In fact, for the purposes of the present invention any agent that can be bound by a binding protein displayed on an EV is considered to be a “small molecule agent”.

When the POI is a viral binding protein the EVs of the present invention are loaded with viral cargos. Exemplary viral cargos include: a viral vector which is an AAV vector or a lentiviral vector.

In some embodiments, the viral vector is an AAV vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes.

In some embodiments, the viral vector is a lentiviral vector. In some embodiments, the lentiviral vector is derived from human immunodeficiency virus, a simian immunodeficiency virus or a feline immunodeficiency virus. In some embodiments, the lentiviral vector is non-replicating. In some embodiments, the lentiviral vector is non-integrating.

In some embodiments the viral vector comprises a viral capsid and a viral genome, the viral genome comprising one or more heterologous transgenes. In preferred embodiments, the heterologous transgene encodes a polypeptide or protein. The protein encoded with in the viral genome may be any one of the protein cargos according to the invention allowing the viral cargo to act as a gene replacement therapy.

In some embodiments the cargo loaded EV may additionally comprise one or more molecules that provide immune effector functions. Immune effector molecules are particularly useful in the case of EVs loaded with a viral (e.g. AAV or lentiviral) cargo but may equally be used where EV is loaded with any cargo according to the invention. The immune effector may act to reduce immunogenicity of the EV. In some embodiments, the immune effector functions stimulate immune inhibitors. In other embodiments, the immune effector functions inhibit immune stimulating molecules. In some embodiments, ABD-EV comprises molecules that stimulate immune inhibitors and molecules that inhibit immune stimulating molecules. Exemplary immune effector molecules include, but are not limited to, one or more of CTLA4, B7-1, B7-2, PD-I, PD-L1, PD-L2, CD28, or VISTA. In some embodiments, the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA.

The immune effector molecule may form part of the TSPAN2 fusion construct, or may form part of a separate fusion protein construct comprising an immune effector molecule fused to any EV protein according to the present invention.

Examples of small molecules include anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or NSAIDs such as naproxen, aspirin, or celecoxib, antibiotics such as heracillin, or antihypertensives such as ACE inhibitors such as enalapril, ARBs such as candesartan, cyclic dinucleotides, etc. The present invention is naturally applicable also to other small molecules without departing from the gist of the invention, as would be clear to a person skilled in the art.

In some embodiments the POI may be a protein capable of binding to an Fc domain, also known as an Fc-binding protein. The terms “Fc binding polypeptide” and “Fc binding protein” and “Fc binder” and “Fc-binding protein” and “binder” are used interchangeably herein and shall be understood to relate to any protein, polypeptide, or peptide (i.e. any molecule comprising a sequence of amino acids) which can bind an Fc domain of any POI. Typically, the Fc binding polypeptides of the present invention are derived from various sources that are either human or non-human (e.g. mammal sources, bacteria, etc.), they have high affinity for Fc domains of various antibody isotypes, subtypes, and species (for instance IgG (as non-limiting examples in the case of IgG, IgG1, IgG2, IgG3, IgG4, IgG2a, IgG2d, and/or IgG2c), IgA, IgM, IgM, IgD, etc.), and they can be fused to EV proteins. Non-limiting examples of Fc binding polypeptides in accordance with the present invention include, in addition to other Fc binding polypeptides mentioned through the present application, Protein A, Protein G, Protein A/G, Z domain, ZZ domain, human FCGRI, human FCGRIIA, human FCGRIIB (as a non-limiting example the accession number 31994), human FCGRIIC (as a non-limiting example the accession number 31995), human FCGRIIIA (as a non-limiting example the accession number P08637), human FCGR3B (as a non-limiting example the accession number 075015), human FCAMR, human FCERA, human FCAR, mouse FCGRI, mouse FCGRIIB, mouse FCGRIII, mouse, mouse FCGRn, and various combinations, derivatives, or alternatives thereof.

The terms “Fc containing protein” and “protein comprising an Fc domain” and “Fc domain-containing protein” and “Fc domain containing protein” and “Fc domain protein” and similar terms are used interchangeably herein and shall be understood to relate to any protein, polypeptide, or peptide (i.e. any molecule comprising a sequence of amino acids) which comprises an Fc domain, either naturally or as a result of engineering of the protein in question to introduce an Fc domain. Fc stands for “fragment crystallizable” or “fragment constant”, which is the name of the tail regions of antibodies. Fc domains can however also be created and used on other proteins, not only antibodies. Non-limiting examples of such Fc domain-containing proteins include antibodies and antibody derivatives, Fc-modified decoy receptors (such as CD24-Fc or CD52-Fc) and/or signal transducers such as interleukin decoy receptors for IL1, IL2, IL3, IL4, IL5, IL6 (such as the signal transducer gp130 (as a non-limiting example the accession number P40189)), IL7, IL8, IL9, IL10, IL1 1, IL12, IL13, IL14, IL15, IL17 (such as IL17R, with as a non-limiting example the accession number Q96F46), IL23 (such as IL23R, with as a non-limiting example the accession number Q5VWK5), etc., Fc domain-containing bi- and multi-specific binders, any type of Fc domain-containing receptors or ligands, Fc domain-modified enzymes for e.g. enzyme replacement therapy or gene editing, nucleases such as Cas and Cas9 onto which an Fc domain has been grafted, tumor suppressors fused to Fc domains, etc. Suitable Fc domains that may be fused with a POI natively lacking an Fc domain include the following non-limiting examples: human IGHM (as a non-limiting example the accession number P01871), human IGHA1 (as a non-limiting example the accession number P01876), human IGHA2 (as a non-limiting example the accession number P01877), human IGKC (as a non-limiting example the accession number P01834), human IGHG1 (as a non-limiting example the accession number P01857), human IGHG2 (as a non-limiting example the accession number P01859), human IGHG3 (as a non-limiting example the accession number P01860), human IGHG4 (as a non-limiting example the accession number P01861), human IGHD (as a non-limiting example the accession number P01880), human IGHE (as a non-limiting example the accession number P01854),

In some embodiments the POI in the present invention is an endosomal escape domain or endsomal escape moiety. In some embodiments the endosomal escape domain may be the POI comprised in the TSPAN2 fusion polypeptide. Alternatively the endosomal escape domain may be present as a second POI expressed on a separate construct.

Endosomal escape domains according to the present invention include: HA2, VSVG, GALA, B18. Other exemplary endosomal escape peptides include: HIV TAT PDT (peptide/protein transduction domain), HIV Gp-120, KALA, GALA and INF-7 (derived from the N-terminal domain of influenza virus hemagglutinin HA-2 subunit), endosomal escape moieties that act by causing membrane fusion such as Diphtheria toxin T domain, proton sponge type endosomal escape moieties such as peptides or lipids with histidine or imidazole moieties and CPPs and other moieties that enable endosomal escape. CPPs are typically less than 50 amino acids but may also be longer, are typically highly cationic and rich in arginine and/or lysine amino acids and have the ability to gain access to the interior of virtually any cell type, exemplary CPPs may be transportan, transportan 10, penetratin, MTS, VP22, CADY peptides, MAP, KALA, PpTG20, proline-rich peptides, MPG peptides, PepFect peptides, Pep-1, L-oligomers, calcitonin peptides, various arginine-rich CPPs, such as poly-Arg, tat and combinations thereof).

The presence of endosomal escape domains advantageously assists to drive endosomal escape and thereby enhance the bioactive delivery of the EV per se. Use of endosomal escape strategies is particularly important in the treatment of diseases where the cargo carried within the EV is required to be delivered into the cytosol of the recipient cell or within any other compartment that is outside of the endo-lysosomal system.

In a further embodiment, the POI may be a targeting moiety. A targeting moiety enables the EV to be engineered so as to allow targeted delivery of the EV to a cell, tissue, organ, and/or compartment of interest. In one embodiment the targeting moiety may be the POI comprised in the TSPAN2 fusion polypeptide. Alternatively the targeting moiety may be present as a second POI expressed on a separate construct. In an advantageous embodiment the targeting moiety is engineered to be displayed on the surface of the EVs.

Targeting moieties may be proteins, peptides, single chain fragments or any other derivatives of antibodies, obtainable from either humans or from non-human animals, etc.

The presence of the targeting moiety combined with the presence of an ABD is especially useful in organ targeting. For example, an EV may comprise TSPAN2 fused to both a therapeutic protein/binding protein for a therapeutic cargo (luminally loaded POI) and a targeting moiety, and on a second construct an ABD fused to a tetraspanin. The POI-TSPAN2-targeting moiety and tetraspanin-ABD constructs are expressed on the same EV and, once that EV is coated in albumin, it can remain in circulation much longer, avoiding uptake by the liver or cells of the immune systems, and thus is able to reach the desired target organ.

Targeting moieties may be used to target the EVs to cell, subcellular locations, tissues, organs or other bodily compartments. Organs and cell types that may be targeted include: the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells.

Targeting can be achieved by a variety of means, for instance the use of targeting peptides. Such targeting peptides may be anywhere from a few amino acids in length to several 100s of amino acids in length, e.g. anywhere in the interval of 3-100 amino acids, 3-30 amino acids, 5-25 amino acids, e.g. 7 amino acids, 12 amino acids, 20 amino acids, etc. Targeting peptides of the present invention may also include full length proteins such as receptors, receptor ligands, etc. Furthermore, the targeting peptides as per the present invention may also include antibodies and antibody derivatives, e.g. monoclonal antibodies, single chain variable fragments (scFvs), nanobodies, other antibody domains, etc.

Exemplary targeting moieties include: brain targeting moieties such as rabies virus glycoprotein (RVG), nerve growth factor (NGF) which binds to NGF-receptor, melanotransferrin and the FC5 Peptide and muscle targeting moieties such as Muscle Specific Peptide (MSP).

The term “albumin binding domain” (ABD) shall be understood to relate to any protein, peptide, antibody or nanobody, or fragment or domain thereof capable of binding to albumin. ABDs may be derived from any species, preferably the ABD has specific binding affinity for human serum albumin. Commonly known ABDs are antibodies or nanobodies that are raised against albumin or ABDs derived from PAB protein from Peptostreptococcus magnus and protein G from group C and G streptococci, both of which bind to albumin with high affinity. Alternatively, the albumin binding domain of the present invention may be an antibody, scFv nanobody, heavy chain antibody (hcAb), single domain antibody (sdAb) such as VHH or VNAR, or a fragment thereof which is capable of binding to albumin. sdAbs and antibody fragments are particularly preferred due to their small size which allows for other additional domains to be introduced into the fusion protein and simple construct generation and expression.

In certain embodiments the EVs of the present invention comprise a second POI expressed on a second construct. Optionally said second POI on the second construct may be present in the form of a fusion protein with TSPAN2 or alternatively a classical EV protein.

Classical EV proteins as per the present invention may be selected from the group comprising the following non-limiting examples: CD9, CD53, CD63, CD81, CD54, CD50, FLOT1, FLOT2, CD49d, CD71, CD133, CD138, CD235a, AAAT, AT1B3, AT2B4, ALIX, Annexin, BASI, BASP1, BSG, Syntenin-1, Syntenin-2, Lamp2, Lamp2a, Lamp2b, TNFR, TfR1, syndecan-1, syndecan-2, syndecan-3, syndecan-4, CD37, CD82, CD151, CD224, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, CLIC1, CLIC4, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD53, CD86, CD110, CD111, CD115, CD117, CD125, CD135, CD184, CD200, CD279, CD273, CD 274, CD362, COL6A1, AGRN, EGFR, FPRP, GAPDH, GLUR2, GLUR3, GP130, GPI anchor proteins, GTR1, HLAA, HLA-DM, HSPG2, ITA3, Lactadherin, L1CAM, LAMB1, LAMC1, LIMP2, MYOF, ARRDC1, ATP2B2, ATP2B3, ATP2B4, BSG, IGSF2, IGSF3, IGSF8, ITGB1, ITGA4, ATP1A2, ATP1A3, ATP1A4, ITGA4, SLC3A2, ATP transporters, ATP1A1, ATP1B3, ATP2B1, LFA-1, LGALS3BP, Mac-1 alpha, Mac-1 beta, MFGE8, a member of the myristoylated alanine rich Protein Kinase C substrate (MARCKS) protein family such as MARCKSL1, matrix metalloproteinase-14 (MMP14), PDGFR, PTGFRN, PRPH2, ROM1, SLIT2, SLC3A2, SSEA4, STX3, TCRA, TCRB, TCRD, TCRG, TFR1, UPK1A, UPK1B, VTI1A, VTI1B, and any other EV proteins, and any combinations, derivatives, domains, variants, mutants, or regions thereof. Mutations may be introduced into the wild type sequence of the EV protein to alter its function, a preferred mutant according to the invention is CD63 (Y235A). The use of EV proteins has the effect of driving loading of the POI into EVs such that POI is actively loaded into EVs, as a result EV-proteins are sometimes referred to as carrier proteins. Particularly preferred classical EV proteins include transmembrane proteins such as tetraspanins, LAMP2B, LIMP2, ICAMs, integrins, ARRDC1, syndecan, syntenin, TNFR, TfR1, and Alix.

As described above the present invention relates to EVs comprising a fusion protein of TSPAN2-POI, TSPAN3-POI or TSPAN18-POI. In other aspects the present invention also relates to the polypeptide construct of TSPAN2-POI, TSPAN3-POI or TSPAN18-POI and any polynucleotide construct encoding said constructs. As described above the TSPAN2/3/18-POI fusion protein may also comprise additional domains including: (i) a release domain capable of cleavage to release the POI; (ii) a linker or spacer and/or (iii) a multimerization domain.

In one advantageous embodiment the TSPAN2/3/18-POI fusion protein further comprises a multimerization domain. Multimerization domains according to the present invention may be homomultimerization domains or heteromultimerization domains. The multimerization domains of the present invention may be a dimerization domain, a trimerization domain, a tetramerization domain, or any higher order of multimerization domain. Multimerization domains enable dimerization, trimerization, or any higher order of multimerization of the fusion polypeptides, which increases the sorting and trafficking of the fusion polypeptides into EVs and may also contribute to increase the yield of vesicles produced by EV-producing cells. Exemplary multimerization domains include: leucine zipper, fold-on domain, fragment X, collagen domain, 2G12 IgG homodimer, mitochondrial antiviral-signaling protein CARD filament, Cardiac phospholamban transmembrane pentamer, parathyroid hormone dimerization domain, Glycophorin A transmembrane, HIV Gp41 trimerisation domain, HPV45 oncoprotein E7 C-terminal dimer domain, and any combination thereof.

In another advantageous embodiment the TSPAN2/3/18-POI fusion protein further comprises a linker, and/or spacer. The presence of a linker, spacer and/or scaffold sequence allows flexibility and enables the POI to be positioned optimally for display on the surface of the EV or luminally according to what is required. Linkers according to the invention are useful in providing increased flexibility, improving pharmacokinetics (PK), increasing expression and improving biological activity of the fusion polypeptide constructs, and also to the corresponding polynucleotide constructs, and may also be used to ensure avoidance of steric hindrance and maintained functionality of the fusion polypeptides. Exemplary linkers according to the present invention include glycine or serine linkers which increase stability or flexibility such as (GGGGS)ₙ (n=1, 2, 4) or (Gly)₆, (Gly)₈, rigid linkers such as (EAAAK)n (n=1-3, and A(EAAAK)4ALEA(EAAAK)4A, bending linkers (XP)ₙ or cleavable linkers such as disulphide, protease sensitive sequences.

Suitable release domains according to the present invention may be cis-cleaving or “self cleaving” sequences such as inteins, light induced monomeric or dimeric release domains such as Kaede, KikGR, EosFP, tdEosFP, mEos2, PSmOrange, the GFP-like Dendra proteins Dendra and Dendra2, CRY2-CIBN, etc. Alternatively, nuclear localization signal (NLS)—nuclear localization signal-binding protein (NLSBP) (NLS-NLSBP) release system may be employed. Protease cleavage sites may also be incorporated into the fusion proteins for spontaneous release, etc., depending on the desired functionality of the fusion polypeptide. In the case of nucleic acid cargos specific nucleic acid cleaving domains may be included. Non-limiting examples of nucleic acid cleaving domains include endonucleases such as Cas6, Cas13, engineered PUF nucleases, site specific RNA nucleases etc.

The inclusion of release domains is highly advantageous because they enable release of particular parts or domains from the original fusion polypeptide. This is particularly advantageous when the release of parts of the fusion polypeptide would increase bioactive delivery of the cargo and/or when a particular function of the fusion polypeptide works better when part of a smaller construct.

The term “self-cleaving protein” can mean a naturally occurring protein that excises itself from a native host protein through self-cleavage. A suitable example of a self-cleaving protein is an intein. It is to be appreciated that certain modifications are desirable to provide a protein that has self-cleaving capability only (i.e., no self-splicing). As suitable example of a protein capable of only self-cleavage (and no splicing) is ΔI-CM.

The term “self-splicing protein” can mean a naturally occurring protein that excises itself from a native host protein through self-splicing and ligation of their flanking peptide bonds. A suitable example of a self-splicing protein is an intein.

The terms “mini-intein” or “delta-intein” are used interchangeably herein and can be understood to relate to a modified intein, preferably from parent RecA, and lacking the endonuclease domain.

The term “Fast-cleaving intein” can be understood to relate to an intein or mini-intein that has been modified at the +C-extein position and/or −N-extein position so that the cleavage rate of said intein is quicker/faster than the original RecA, intein, or mini-intein.

The term “Slow-cleaving intein” can be understood to relate to an intein or mini-intein that has been modified at the +C-extein position and/or −N-extein position so that the cleavage rate of said intein is slower than the original RecA, intein, or mini-intein. An example of a slow-cleaving intein is ΔI-CM.

The cis-cleaving sequence may be a self-cleaving protein, for example an intein. The intein may be a slow-cleaving or a fast-cleaving intein. The intein may be a mini-intein, such as a mini-intein that has been modified to optimise the cleavage rate. The intein may be a delta-intein-CM. Thus, cis-cleaving sequences (or self-cleaving) in accordance with the disclosure may include, but are not limited to, the following:

Inteins, mini-inteins, delta inteins and certain variants, mutations and domains thereof having a desired functionality (including, but not limited to self-cleaving instead of splicing), such as a mini-intein modified to optimise the cleavage rate. For example, a mini-intein having C1A, D24G, V67L and/or D150G substitution in the N-terminal portion (or an appropriate—C-extein position). Said suitable substitutions can be found in SEQ ID No. 20. For example, splicing is enabled by the +1 position of the intein, wherein the +1 is Cys. Substitution of Cys with Ala in ΔI-CM removes the splicing capability and supports cleavage only. It will be appreciated that other mutations, substitutions, and the like may also similarly work. As such the substitution hereinbefore mentioned, while exemplary, is not limiting in any way and may also differ from intein to intein.

In certain instances, one may opt to utilize slow-cleaving inteins. A slow-cleaving system may be preferable when more time is required to ensure efficient loading of an EV with the desired cargo. Slow-cleaving inteins in accordance with the disclosure may include, but are not limited to, the following: mini-inteins, delta inteins, delta-intein-CM and a mini-intein and certain variants, mutations and domains thereof having a desired functionality (such as, but not limited to cleavage action instead of splicing and cleavage rate). In a preferred embodiment, the slow-cleaving cis-cleaving release system is based on an intein system, wherein the C-terminal portion of the intein may comprise the amino acid sequences Val-Val-Val-His-Asn, more preferably wherein the C-terminal portion of the intein is modified to comprise Val-Val-Val-His-Asn-Gly. Certain modifications at the +1 C-extein position, have been observed to slow the cleavage rate (i.e., are slow-cleaving).

In certain instances, one may opt to utilize a fast-cleaving cis-cleaving release system (such as a fast-cleaving cis-cleaving intein). A fast-cleaving system may be preferable when EVs need to be harvested quickly. In a preferred embodiment, the fast-cleaving cis-cleaving release system is based on an intein system, wherein the C-terminal portion of the intein may comprise the amino acid sequences Val-Val-Val-His-Asn or Val-Val-Val-His-Asn-Cys. Certain modifications at the +1 C-extein position, such as the abovementioned example, have been observed to speed up the cleavage rate (i.e., are fast-cleaving).

In a specific embodiment the EV of the invention comprises more than one POI. This may be as a result of more than one POI being present in a single TSPAN2 fusion protein, alternatively it may be as a result of multiple fusion proteins being loaded into a single EV. Said multiple fusion proteins may comprise the same or different scaffold protein and the same or different POI. The presence of more than one POI on a single EV is advantageous as it increases the therapeutic effect of the EV and, where the POIs are different, allows multiplexing of cargos to improve the delivery, therapeutic effect, targeting etc.

In certain embodiments the POI cargo carried by the EVs may be present on the inside of the EV, on the outside of the EV or in the membrane of the EV. The desired location of the therapeutic cargo will depend on the nature of the cargo and its mechanism of action, for instance a membrane protein will preferably be located in the membrane of the EV, a decoy receptor, endosomal escape moiety or targeting moiety will preferably be present on the surface of the EV, but a cargo designed to be delivered into the cytosol or nucleus of the recipient cell, such as a silencing RNA or AAV, will preferably be located inside the lumen of the EV. The design of the fusion proteins of the invention allows for expression on the surface or in the lumen of the EV.

The fusion of POIs to TSPAN2 or other classical EV proteins as described above shall be understood to be endogenous loading of said EVs. In alternative embodiments therapeutic cargos may be passively loaded into EVs or actively loaded by exogenous loading methods as described below.

The therapeutic cargo may be loaded passively into the EVs by the therapeutic cargo being present in the cytosol of the EV producing cells. Such passive loading applies, for instance, to nucleic acids, small molecules, viruses, soluble proteins or membrane proteins that are naturally loaded into the EVs.

In certain embodiments the therapeutic cargo is actively loaded into the EVs of the present invention. One form of active loading of cargos involves exogenous active loading which involves cargo being loaded using any known exogenous loading method including: electroporation, transfection with transfection reagents such a cationic transfection agents, lipofectamine (RTM), conjugation of the cargo to a membrane anchoring moiety such as a lipid or cholesterol tail or loading by means of a CPP, either in the form of a CPP-cargo conjugate or in the form of a CPP-cargo non-covalent complex or any combination of these methods. Again, this type of active loading may result in the therapeutic cargo being located on the inside of the EV, on the outside of the EV or located within the membrane of the EV. Any of the cargos as defined above may be loaded exogenously. Particularly preferred embodiments involve exogenous loading of nucleic acid or viral cargos by electroporation, CPP loading or co-incubation with a lipid tagged cargo.

The present invention also relates to a population of EVs comprising at least one TSPAN2-POI fusion protein. Additionally, the present invention also relates to a population of EVs comprising at least one TSPAN2-POI fusion protein and a second POI construct being expressed on the same EV. As detailed above a major advantage of using TSPAN2 as the scaffold protein for loading POIs into engineered EVs is that is enables reliable and sustained co-localization of additional constructs onto the same EV.

In certain embodiments the average number of POIs per EV in the population of EVs according to the invention is above one POI per EV, but it may also be below one per EV. Furthermore, in certain embodiments, in the population of EVs according to the invention, the average number of POIs per EV is above or below one POI per EV. In another embodiment, in the population of EVs according to the invention at least 5%, at least 10%, at least 20%, at least 50%, at least 70%, at least 75%, at least 80%, at least 85%, at least 90%, and/or at least 95% of all EVs comprise at least one TSPAN2-POI construct and optionally also at least one further POI.

Generally, EVs may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method, as well as any adult cell source. The source cells per the present invention may be select from a wide range of cells and cell lines, for instance mesenchymal stem or stromal cells (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, erythrocytes, erythroid progenitors, chondrocytes, mesenchymal stromal cells (MSCs) of different origin, amnion cells, amnion epithelial (AE) cells, CEVEC's CAP® cells any cells obtained through amniocentesis or from the placenta, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. The source cell may be either allogeneic, autologous, or even xenogeneic in nature to the patient to be treated, i.e. the cells may be from the patient himself or from an unrelated, matched or unmatched donor.

In particular the present invention relates to cells which have been stably modified to comprise at least one monocistronic, bicistronic or multicistronic polynucleotide construct according to the invention (as defined above) encoding a fusion protein of TSPAN2-POI. Such cells may be stably or transiently transfected with the polynucleotides according to the present invention to render them engineered EV producing cells. Such cells may also be stably or transiently modified so as to include a second construct encoding for a second POI which optionally may form part of a fusion protein with a classical EV protein. The cells of the present invention may be a monoclonal cell or a polyclonal cell line.

Preferred producer cells according to the present invention may be a: HEK cell, a HEK293 cell, a HEK293T cell, an MSC, in particular a WJ-MSC cell or a BM-MSC cell, a fibroblast, an amnion cell, an amnion epithelial cell, CEVEC's CAP® cells, a placenta-derived cell, a cord blood cell, an immune system cell, an endothelial cell, an epithelial cell or any other cell type, wherein said cells may be for instance adherent cells, suspension cells, and/or suspension-adapted cells. In a more preferred embodiment, the producer cells are HEK293 cells. In an alternative more preferred embodiment, the producer cells are CAP cells.

Accordingly, in a preferred embodiment the producer cells are HEK293 cells or CAP cells and the polynucleotide construct encodes a fusion protein of TSPAN2-POI.

The present invention also relates to pharmaceutical compositions comprising at least one EV according to the invention and a pharmaceutically acceptable excipient or carrier.

The term “excipient” or “carrier” refers to an inert substance added to a pharmaceutical composition to further facilitate administration of a compound. The terms encompass any of the agents approved by a regulatory agency such as the FDA or EMEA or listed in the U.S. Pharmacopeia for use in animals, including humans, as well as any carrier or diluent that does not cause significant irritation to a subject and does not abrogate the biological activity and properties of the therapeutic cargo. Included are excipients and carriers that are useful in preparing a pharmaceutical composition and are generally safe and non-toxic.

Exemplary excipients include degradation or loss of activity stabiliser excipients such as proteins such as human serum albumin, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca²⁺, Zn²⁺ and EDTA, Cyclodextrin based such as hydroxypropyl β-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol.

The pharmaceutical compositions according to the present invention may be formulated by any known method of formulation including but not limited to:
Oral formulations—Tablet, Capsule, Sustained release, liquid
Intravenous formulations
Parenteral formulations
Topical formulations—cutaneous administration: cream, ointment, gel, paste, powder
Modified release formulations—sustained release formulation
Liquid or lyophilized formulations

The EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the cargo molecule in question, or the EV population as such.

It will be clear to the skilled artisan that when describing medical and scientific uses and applications of the EVs, the present invention normally relates to a plurality of EVs, i.e. a population of EVs which may comprise thousands, millions, billions or even trillions of EVs. EVs may be present in concentrations such as 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume or per unit of weight (for instance per ml or per L or per kg of body weight), or any other number larger, smaller or anywhere in between. In the same vein, the term “population”, which may e.g. relate to an EV comprising a certain cargo shall be understood to encompass a plurality of entities constituting such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the cargo of interest, any targeting moieties present on the EVs, the pharmaceutical formulation, etc.

It is envisaged that any dosage regime would be applicable to the engineered EVs of the invention. The dosage regime chosen will depend on the cargo being delivered by the EVs and the disease to be treated and any additional therapies being administered which will be determined by the skilled physician.

It is envisaged that the EVs of the present invention will be administered multiple times, i.e. more than 1 time but normally more than 2 times or potentially for chronic, long-term treatment (i.e. administered tens to hundreds to thousands of times). Preferably, if the cargo is an antigen that is being administered as a vaccine, the immunization schedule will involve two or more administrations of the polypeptide, spread out over several weeks. Similarly, if the cargo is e.g. an RNA agent such as an siRNA or mRNA or a protein such as an antibody or an enzyme or a transporter, the EVs comprising the cargo in question will likely be administered more than once, normally multiple times as part of a chronic treatment regimen.

The present invention relates to a method for producing the EVs according to the invention. The method for producing EVs comprises: (i) introducing into an EV-producing cell a polynucleotide construct encoding a TSPAN2-POI fusion construct; and (ii) expressing the construct in the EV-producing cell, thereby generating EVs comprising the TSPAN2-POI fusion protein.

The method for producing EVs according to the present invention may further comprise a second step of introducing into the same EV producing cell a second polynucleotide construct encoding a second POI, optionally wherein the second POI is present in the form of a fusion protein with TSPAN2 or a classical EV protein. The POI may naturally be any POI as defined above.

The methods described above to generate fusion proteins and EVs comprising fusion proteins can be achieved by protein and cell engineering techniques which are commonly known in the art, such as molecular cloning and transfection of cells with vectors encoding fusion constructs.

The method for producing the EVs may further comprise a step of exogenously loading the EV with a cargo molecule. As described above the exogenous loading step may comprise loading of the cargo by any exogenous loading method including: electroporation, microfluidics, transfection with transfection reagents such a cationic transfection agents, lipofectamine (RTM), conjugation of the cargo to a membrane anchoring moiety such as a lipid or cholesterol tail or loading by means of a CPP, either in the form of a CPP-cargo conjugate or in the form of a CPP-cargo non-covalent complex. Particularly preferred embodiments involve methods of exogenous loading of nucleic acid or viral cargos by electroporation, CPP loading or co-incubation with a lipid tagged cargo.

The method for producing engineered EVs according to the present invention may further include a step of purification of the EVs. Purification of EVs is achieved by any method including but not limited to: techniques comprising liquid chromatography (LC), high-performance liquid chromatography (HPLC), bead-eluate chromatography, ionic exchange chromatography, spin filtration, tangential flow filtration (TFF), hollow fiber filtration, centrifugation, immunoprecipitation, flow field fractionation, dialysis, microfluidic-based separation, etc., or any combination thereof. In an advantageous embodiment, the purification of the EVs is carried out using a sequential combination of filtration (preferably ultrafiltration (UF) tangential flow filtration (TFF) or hollow fibre filtration) and affinity chromatography, optionally also including size exclusion LC or bead-eluate LC. Combining purification steps normally enhances the purity of the resulting samples and, in turn leads to superior therapeutic activity. Further, as compared to UC, which is routinely employed for purifying exosomes, sequential filtration-chromatography is considerably faster and possible to scale to higher manufacturing volumes, which is a significant drawback of the current UC methodology that dominates the prior art. Another advantageous purification method is TFF, which offers scalability and purity, and which may be combined with any other type of purification technique.

Alternatively, the present invention also relates to a method of producing EVs in a patient, (i.e. patient-derived engineered EVs), the method comprising administering a delivery vector comprising a polynucleotide according to the invention to the patient, wherein target cells in the patient produce EVs comprising the TSPAN2-POI or TSPAN3-POI or TSPAN18-POI fusion protein, optionally the delivery vector may also comprise a polynucleotide which encodes for a second construct comprising a POI which is also co-expressed on the same patient derived EVs as the TSPAN2-POI construct. In a preferred embodiment the polynucleotide is mRNA, circular mRNA, dbDNA®, linear DNA, circular DNA, plasmid DNA, linear RNA, circular RNA, self-amplifying RNA or DNA, a viral genome either “naked” or within a capsid or a modified version of any of the above. In a preferred embodiment the delivery vector is a viral vector or a non-viral vector selected from the group consisting of a lipid nanoparticle (LNP), a virus like particle (VLP), a CPP, a polymer or a pharmaceutically acceptable carrier.

In one embodiment the present invention therefore also relates to a delivery vector comprising a polynucleotide according to the invention, wherein the polynucleotide cargo codes for a fusion protein comprising a POI and is arranged to be translated into the fusion protein by an EV-producing cell, said translation resulting in the production of at least one EV comprising the TSPAN2-POI, TSPAN3-POI or TSPAN18-POI fusion protein, optionally wherein the delivery vector also comprises a polynucleotide which encodes for a second construct comprising a POI which is also co-expressed on the same patient derived EVs as the TSPAN2-POI construct. Such TSPAN2/3/18-POI EVs derived from patients will have all the same benefits of reliability of construction expression and ability to co-express multiple constructs etc as the bioreactor produced EVs described above.

The present invention also relates to EVs according to the invention for use in medicine.

The present invention also relates to a method of treatment comprising administering to a patient in need thereof, an effective amount of EVs according to the invention or a pharmaceutical composition of the invention.

The use in medicine or method of treatment (collectively “the treatment”) may be by delivery of any kind of cargo according to the invention. For instance the treatment may be by delivery of functional proteins as protein replacement therapy, delivery of mRNA encoding for functional proteins to also act as a protein replacement therapy. Such a protein replacement therapy may, for instance, be an enzyme replacement therapy (ERT) for diseases caused by inborn errors in metabolism such as Phenylketonuria, urea cycle disorders, or lysosomal storage disorders. The treatment may be by delivery of: gene silencing RNAs, splice switching RNAs, or CRISPR-Cas9 for gene editing. The treatment may be gene therapy by delivery of plasmid DNA, mini-circles or viral gene therapies such as AAVs or lentiviruses. The treatment may be by presentation of an antigen or neoantigen for immunotherapy, in effect acting as a vaccine to induce an immune response. For instance the EV may act by delivery and/or presentation of a tumour antigen for cancer immunotherapy, or viral, bacterial or fungal antigens for immunization against pathogens. The treatment may be by delivery of small molecules, antibodies and antibody-drug conjugates capable of mediating a therapeutic effect once delivered into a cell or the extracellular matrix. In one embodiment, the treatment or therapy may be effected by the EVs comprising more than one type of therapeutic cargo, i.e. the therapeutic cargo may be a mixture of protein, nucleic acid, virus, viral genome, antigen and/or small molecule.

Importantly, the present invention relates to use of the EV composition described herein in the prophylaxis and/or treatment and/or alleviation of a variety of diseases, typically via the delivery of essentially any type of drug cargo, such as for instance: a nucleic acid such as an RNA molecule, a DNA molecule or a mixmer, mRNA, antisense or splice-switching oligonucleotides, siRNA, shRNA, miRNA, plasmid DNA (pDNA), supercoiled or unsupercoiled plasmids, mini-circles, peptides or proteins including: transporters, enzymes, receptors such as decoy receptors, membrane proteins, cytokines, antigens and neoantigens, ribonuclear proteins, nucleic acid binding proteins, antibodies, nanobodies, antibody fragments, antibody-drug conjugates, small molecule drugs, gene editing technology such as CRISPR-Cas9, TALENs, meganucleases, or vesicle-based cargos such as viruses (e.g. AAVs, lentiviruses, etc.). In one embodiment, the cargo may be a mixture of protein, nucleic acid, virus, viral genome, antigen and/or small molecule.

Non-limiting examples of diseases and conditions that are suitable targets for treatment using the peptide delivery system described herein include the following non-limiting examples: autoimmune diseases (such as celiac disease, Crohn's disease, diabetes mellitus type 1, Graves' disease, inflammatory bowel disease, multiple sclerosis, psoriasis, rheumatoid arthritis, systemic lupus erythematosus) ulcerative colitis, ankylosing spondylitis, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barre syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy and other muscular dystrophies, In-born errors of metabolism including: Disorders of carbohydrate metabolism e.g., G6PD deficiency galactosemia, hereditary fructose intolerance, fructose 1,6-diphosphatase deficiency and the glycogen storage diseases, Disorders of organic acid metabolism (organic acidurias) such as alkaptonuria, 2-hydroxyglutaric acidurias, methylmalonic or propionic acidemia, multiple carboxylase deficiency, Disorders of amino acid metabolism such as phenylketonuria, maple syrup urine disease, glutaric acidemia type 1, Aminoacidopathies e.g., hereditary tyrosinemia, nonketotic hyperglycinemia, and homocystinuria, Hereditary tyrosinemia, Fanconi syndrome, Primary Lactic Acidoses e.g., pyruvate dehydrogenase, pyruvate carboxylase and cytochrome oxidase deficiencies, Disorders of fatty acid oxidation and mitochondrial metabolism such as short, medium, and long-chain acyl-CoA dehydrogenase deficiencies also known as Beta-oxidation defects, Reye's syndrome, Medium-chain acyl-coenzyme A dehydrogenase deficiency (MCADD.), MELAS, MERFF, pyruvate dehydrogenase deficiency, Disorders of porphyrin metabolism such as acute intermittent porphyria, Disorders of purine or pyrimidine metabolism such as Lesch-Nyhan syndrome, Disorders of steroid metabolism such as lipoid congenital adrenal hyperplasia, congenital adrenal hyperplasia, Disorders of mitochondrial function such as Kearns-Sayre syndrome, Disorders of peroxisomal function such as Zellweger syndrome and neonatal adrenoleukodystrophy, congenital adrenal hyperplasia or SmithLemli-Opitz, Menkes syndrome, neonatal hemochromatosis, Urea cycle disorders such as N-Acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), all of the lysosomal storage diseases, for instance Alpha-mannosidosis, Betamannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Mucopolysaccharidosis, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, etc. cystic fibrosis, primary ciliary dyskinesia, pulmonary alveolar proteinosis, ARC syndrome, Ret syndrome, neurodegenerative diseases including Alzheimer's disease, Parkinson's disease, GBA associated Parkinson's disease, Huntington's disease and other trinucleotide repeat-related diseases, prion diseases, dementia including frontotemporal lobe dementia, ALS, motor neuron disease, multiple sclerosis, cancer-induced cachexia, anorexia, diabetes mellitus type 2, and various cancers.

Specifically, it is envisaged that the present invention would be useful in the treatment of cancer by cancer immunotherapy i.e. the presentation of cancer antigens on the surface of EVs so that those antigens raise an immune response against the cancer antigen. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The present invention is also specifically advantageous for the treatment of brain and CNS disorders due to the ability to target TSPAN2 expressing EVs to the brain whilst consistently also expressing another POI such as a therapeutic protein or endosomal escape moiety. The present invention specifically relates to EVs with ABD present on the surface of the EV which are loaded with any type of cargo according to the invention but preferably an nucleic acid cargo such as silencing RNAs such as siRNAs which target RNAs known to be involved with neurodegenerative diseases such as Alzheimer's disease, Parkinson's disease, Huntington's disease, Spinocerebellar ataxia, amytrophic Lateral Sclerosis (ALS), Frontal temporal Dementia, motor neuron disease, multiple sclerosis, Wallerian Degeneration and Bullous Retinoschisis, Goldberg-Shprintzen Syndrome, Kuru, Autoimmune GFAP Astrocytopathy, MECP2 duplication syndrome, AQP4-astrocytopathy, Familial pain syndromes such as erythromelalgia, paroxysmal extreme pain disorder and congenital insensitivity to pain, Pelizaeus-Merzbacher disease, prion diseases including Creutzfeldt-Jakob disease (CJD), Gerstmann-Sträussler-Scheinker syndrome (GSS), and fatal familial insomnia (FFI), Leukodystrophies including Demyelinating, Adult-Onset, Autosomal Dominant and Leukodystrophy.

The present invention specifically relates to EVs loaded with therapeutic cargos which may be either protein and/or nucleic acid cargos, for the treatment of Duchenne Muscular Dystrophy (DMD), diseases caused by in-born errors of metabolism including lysosomal storage disorders including Niemann Pick Type C (NPC) and Pompe disease, urea cycle disorders such as Argininosuccinic aciduria (ASA) and citrullinemia and Ornithine transcarbamylase (OTC) deficiency, metachromatic leukodystrophy and phenylketourea (PKU).

Wherein the POI is a binding protein such as albumin binding protein, an Fc binder, a nucleic acid binding protein, viral binding protein or small molecule binding protein, the present invention is also directed to nanoparticle complexes comprising any TSPAN2 EVs according to the invention bound to the corresponding binding partner (such as albumin, an Fc containing protein, a nucleic acid, a virus or a small molecule). In this embodiment some or all of the EVs are bound to their corresponding binding partner. The present invention also relates to pharmaceutical compositions comprising the nanoparticle complexes of the invention combined with a pharmaceutically acceptable excipient or carrier.

### EXAMPLES

### Example 1—Establishment of a High-Throughput Screening Protocol

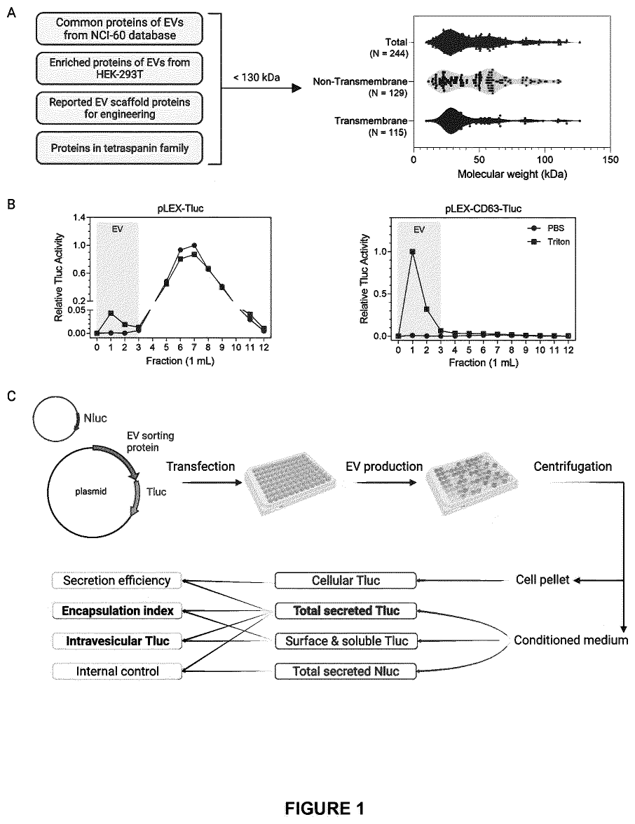

To date, only a few scaffold domains have been used for endogenous loading of protein therapeutics into EVs. To expand the range of scaffold domains available, the present inventors compiled a list of potential EV scaffold domains based on a literature review and proteomics databases (FIG. 1A). The candidates were selected but not limited to the following criteria: (1) common EV proteins from the NCI-60 database; (2) proteins enriched in EVs derived from human embryonic kidney-(HEK-) 293T cells; (3) reported EV scaffold proteins used as reference and (4) Proteins from the tetraspanin family with as yet no known function/association with EVs. This fourth set of proteins were included because it was hypothesized that due to extensive use of CD9, CD63 and CD81 for genetic engineering other proteins in the tetraspanin family might also exhibit high loading efficiencies (FIG. 1A). Of note, all proteins with a molecular weight larger than 130 kDa were excluded. Finally, a total of 244 candidates with a medium size of 38 kDa were included of which 115 were transmembrane proteins (FIG. 1A).

To be able to screen all these domains in a high-throughput manner, the present inventors opted for a reporter system based on bioluminescence using ThermoLuc because it is a sensitive and stable reporter for labelling engineered EVs. A proof-of-concept study was performed to ensure that the Tluc-based assay can discriminate intravesicular cargo from the rest (surface and soluble fraction). HEK-293T cells were transfected with plasmids encoding Tluc alone or fused to CD63, and their conditioned media were fractionated using size exclusion chromatography (SEC). Tluc activity was quantitated in each fraction in the presence or absence of the detergent Triton. As expected, Tluc activity was primarily detected in the soluble fraction for conditioned medium of Tluc-transfected cells but shifted towards the EV fraction when fused to CD63. Moreover, Tluc activity was only detected after membrane disruption with Triton, indicating its intravesicular location (FIG. 1B) and thus the feasibility of our approach. For comparative analysis of EV-sorting ability, Tluc was fused to the C-termini of the 244 EV scaffold domains and the resulting fusion cassettes were cloned into mammalian expression plasmids. These plasmids were then introduced into HEK-293T cells in a microplate format. Minor amounts of plasmids encoding another highly sensitive luciferase, i.e., NanoLuc (Nluc) were spiked into the transfection mixture as an internal control to adjust for confounders such as transfection efficiency. For downstream analyses, the candidates were primarily compared in terms of total secreted Tluc, intravesicular Tluc (total secreted Tluc subtracted by surface/soluble Tluc,) and encapsulation index (the ratio of total secreted Tluc versus surface/soluble Tluc).

FIG. 1 shows the high-throughput screening scheme. (1A) Selection and overview of potential domains for EV-sorting. (1B) SEC elution profile of Tluc and CD63-Tluc medium. HEK-293T cells were transfected with plasmids encoding standalone Tluc or CD63-Tluc fusion protein and the conditioned media were fractionated. Tluc activity in each fraction was quantified with or without membrane lysis using Triton. (1C) Experimentation and data analysis scheme. HEK-293T cells were grown in 96-well plate and co-transfected with 0.75 μg/mL fusion plasmid and 7.5 ng/ml Nluc plasmid for 48 hr. The samples were then centrifuged and Tluc activity was measured in the cell pellet and conditioned medium. Nluc activity was only quantified in the conditioned medium.

### Example 2—Validation of Tluc High-Throughput Screening Platform

To ascertain the robustness of our high-throughput screening platform, all candidates were ranked according to total secreted Tluc as well as the ratio of total secreted Tluc/Nluc.

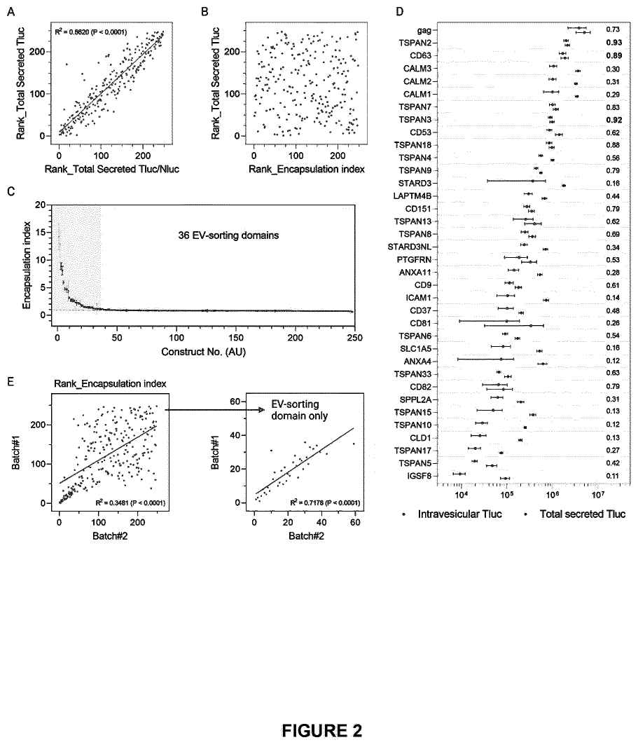

FIG. 2 shows the results of screening for Tluc in the secretome of HEK-293T cells. (2A) Correlation between the rank of total secreted Tluc and the rank of total secreted Tluc/Nluc. (2B) Correlation between the rank of total secreted Tluc and the rank of encapsulation index. (2C) Overview of encapsulation index. Candidates with an encapsulation index above one (the dash line) were considered EV-sorting domains. N=5. (2D) Total secreted and intravesicular Tluc for the 36 EV-sorting domains. The figure indicates the ratio of intravesicular versus total secreted Tluc. N=5. (2E) Correlation of the rank of encapsulation index obtained from two independent batches. Each dot in the scatter plots refers to one candidate and the red dot marks the reference EV-sorting domain CD63.

The results demonstrate that normalization against secreted Nluc did not affect the relative secretion of Tluc (FIG. 2A), thus transfection efficiency was not assumed to be a major confounder. The presence of the three well-studied EV-sorting tetraspanins (CD9, CD63 and CD81) and two reported domains (gag and PTGFRN) within this subset was suggestive of a robust utility of our screening protocol (FIG. 2D). The present inventors found that the candidates exhibited heterogenous EV-sorting ability as seen from the poor correlation between the rank of encapsulation index and the rank of total secreted Tluc (FIG. 2B). Indeed, only 36 domains had an encapsulation index above one, which were consequently deemed promising EV-sorting domains in HEK-293T cells (FIG. 2C). The poor correlation observed and the fact that only 36 out of 244 promising candidates were effective is indicative of the fact that it is hard to predict which carrier/scaffold proteins will be useful in loading POIs into EVs without extensive screening and inventive effort.

Noticeably, more than 90% of total secreted Tluc was inside EVs for both TSPAN2 and TSPAN3, with TSPAN2 loading even more intravesicular Tluc than CD63. The reproducibility of the screening was assessed by comparing the rank of candidates in regard to total secreted Tluc or secretion efficiency (the ratio of total secreted Tluc versus cellular Tluc). The linear correlation between two independent batches as well as different doses well exemplified high reproducibility. Importantly, regarding the rank of encapsulation efficiency, the overall correlation between two independent batches was rather poor. However, looking only at the most promising 36 EV-sorting domains (FIG. 2E) correlation increases drastically, which underlines the reliability of those domains.

### Example 3—Validation of Sorting Ability to Small EVs

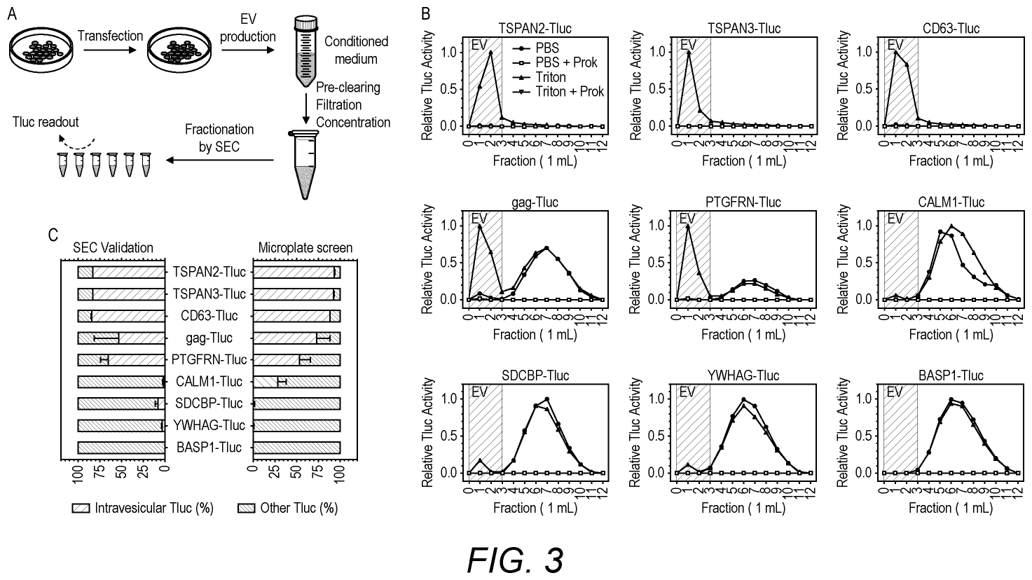

On the basis of the widespread EV-sorting efficiency obtained from the microplate-based screen, a few representative and/or promising candidates were further examined using SEC. Different to the screening protocol, the conditioned medium was additionally filtered through a 200 nm membrane and concentrated to enrich for small EVs prior to SEC fractionation (FIG. 3A). Besides using Triton to distinguish intravesicular Tluc from others, Proteinase K (ProK) was also spiked into each fraction to discern resistant protein aggregates (FIG. 3B). In line with the exceptional EV-sorting ability found in the screen, TSPAN2 and TSPAN3 ranked top in terms of percentage of intravesicular Tluc to total secreted Tluc showing improved activity over CD63 (>80%; FIG. 3C).

FIG. 3 shows the results of the screen for the ability to sort to small EVs. (3A) Validation scheme using size exclusion chromatography (SEC). HEK-293T cells were grown in 15-mm petri-dishes and transfected with 1.5 μg/mL plasmid for 6 hr. The medium was changed to Opti-MEM and collected 48 hr later. Following centrifugation, filtration through 0.2 μm membrane and subsequent concentration, the samples were fractionated and Tluc activity in each fraction was quantified in the presence/absence of Triton or Proteinase K. (3B) Relative Tluc activity in SEC fractions of conditioned medium. (3C) Percentage of intravesicular Tluc to total secreted Tluc. Validation data were from two independent experiments.

TSPAN2 and TSPAN3 ranked top (80%) and fourth (45%), respectively, in terms of percentage of intravesicular Tluc, whereas CD63 only sorted 28% Tluc into EVs. Consistent with the results from HEK-293T cells, SDCBP, CALM1, YWHAG, or BASP1 showed poor EV-sorting efficiency.

### Example 4—Quantification of Engineered Small EVs at Single-Vesicle Level

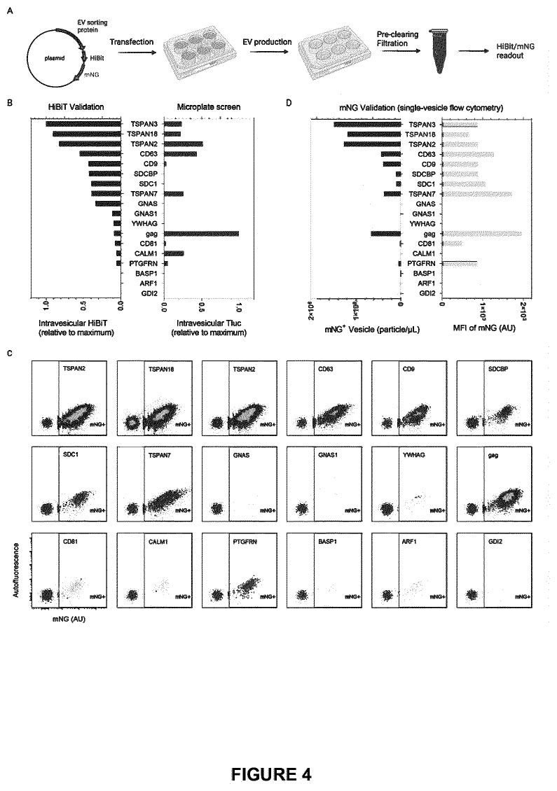

To analyze EVs in more detail and obtain information about the concentration and relative abundance of certain transgene molecules (through mean fluorescence intensity, MFI) per EV, single-vesicle flow cytometry was the method of choice. Luciferase is a useful reporter to quantify engineered EVs in bulk. Tluc was replaced with a hybrid reporter consisting of the 11-mer peptide HiBIT (1.3 kDa) and the fluorescent protein mNeonGreen (mNG; 26.6 kDa) (FIG. 4A). HiBiT can complement its chaperone subunit LgBiT and form a fully functional luciferase complex (Nluc) which enables the comparison of the amount of engineered EVs to the results from the Tluc-based screening. A total of 18 domains were investigated, including the top five domains in terms of encapsulation index, total secreted Tluc or secretion efficiency from the screening readout, three EV markers (CD9, CD81, SDCBP), PTGFRN and CALM1.

FIG. 4 shows the results of this validation using a bifunctional bioluminescence and fluorescence reporter. (4A) Validation scheme using the hybrid HiBiT-mNG reporter. HEK-293T cells were grown in 6-well plate and transfected with 1.5 μg/mL plasmid for 6 hr. The medium was then changed to Opti-MEM and collected 48 hr later. Following centrifugation and filtration through 0.2 μm membrane, the samples were quantified in terms of intravesicular HiBiT activity and mNG fluorescence. (4B) Comparison of intravesicular HiBiT versus Tluc. 4(C) Single-vesicle flow cytometry analysis of mNG-tagged small extracellular vesicles. (4D) Quantification of the concentration and mean fluorescence intensity of mNG-tagged small extracellular vesicles.

TSPAN3, TSPAN2 and CD63 consistently exhibited high EV-sorting ability as demonstrated by high level of intravesicular HiBiT and encapsulation index (FIG. 4B). Furthermore, results from flow cytometry showed that TSPAN3 and TSPAN2 produced the highest number of engineered EVs, around 3.5-fold more than CD63 (FIG. 4C; FIG. 4D). Interestingly, using the above described bifunctional screen TSPAN18 also emerged as an efficient EV-sorting domain, being only secondary to TSPAN3.

Removal of large particles resulted in negligible concentration of engineered EVs for the domain CALM1. Calmodulins appear to be ideal domains for sorting cargo molecules into large EVs. This discovery would be useful for applications employing large EVs for drug/gene delivery of larger cargos.

### Example 5—Tluc Screening Results in Range of Different Cell Lines

The present inventors sought to identify EV scaffold proteins that are highly effective not simply in a single producer cell line but that are effective across multiple different cell lines such that they may be used in a versatile way as a platform technology rather than with narrow functionality. To test the EV sorting ability four more cell lines were tested including: Freestyle 293-F, MSC (mesenchymal stem cell), Huh-7 (human hepatocyte) and TCMK-1 (mouse kidney epithelial cells) were investigated. Based on the screen in the HEK-293T cells, the present inventors tested the top 95 domains, measuring encapsulation index, total secreted Tluc, intravesicular Tluc or secretion efficiency. Concurrent with the findings on HEK-293T cells, transfection efficiency did not seemingly confound the performance of the domains.

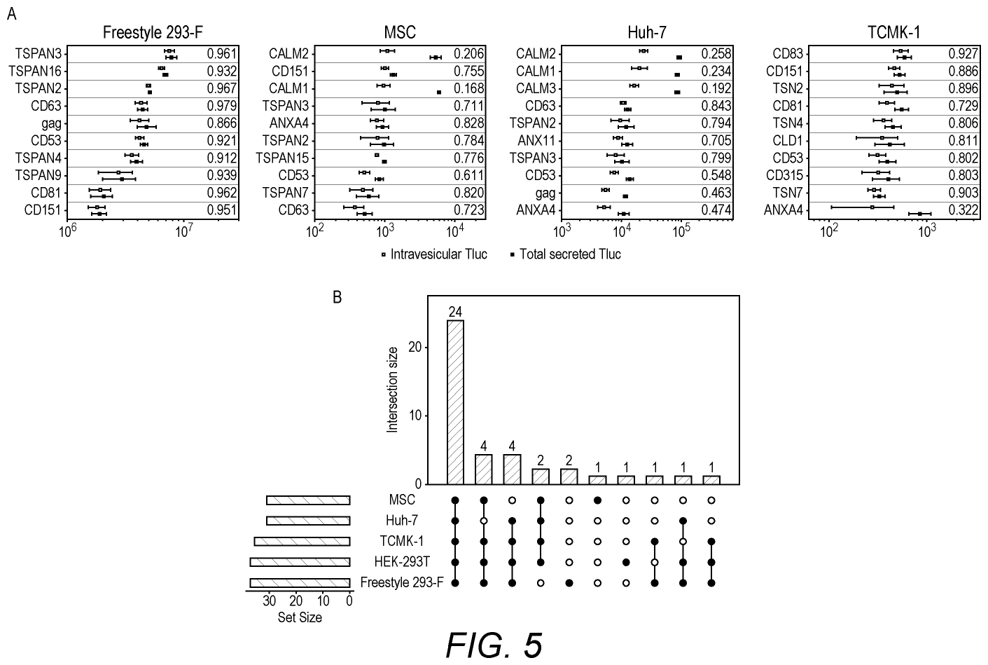

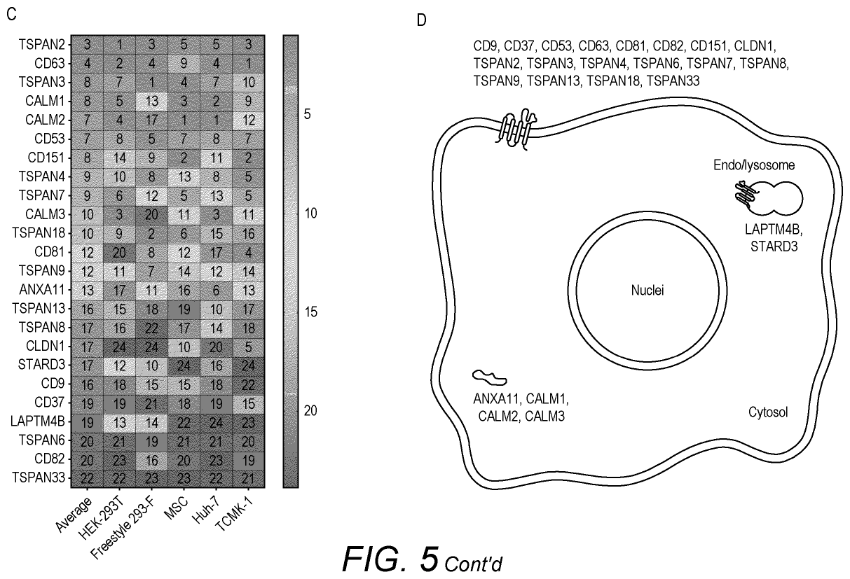

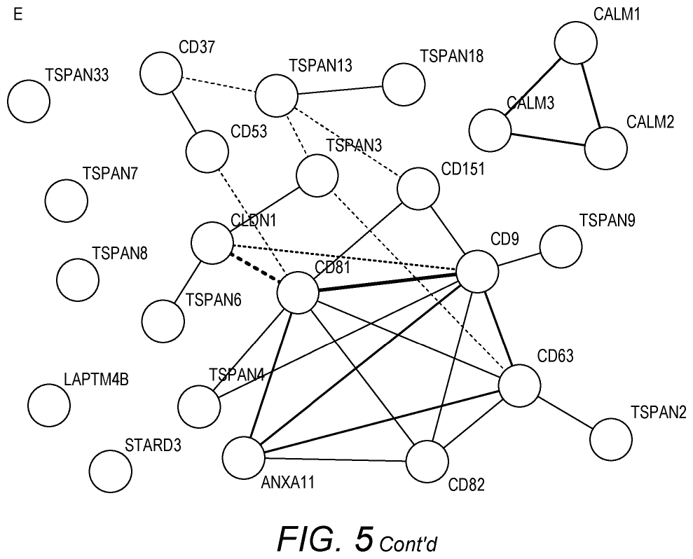

FIG. 5 shows the results of assessing Tluc in the secretome of other EV producer cell lines. (5A) The top ten scaffold domains regards intravesicular Tluc in different producer cell line. N=3. (5B) Number of EV-sorting domains in each producer cell line and across several cell lines. (5C) Relative rank of the 24 conserved EV-sorting domains regards intravesicular Tluc in each cell line as well as the average thereof. (5D) Subcellular location of the 24 conserved EV-sorting domains. (5E) Interaction network of the 24 conserved EV-sorting domains. Line thickness indicates the strength of data support.

TSPAN2 sorted the highest amount of Tluc into EVs in Freestyle 293-F and TCMK-1 cells, respectively, while CALM2 was the best domain in MSC and Huh-7 cells (FIG. 5A). Overall, for the five cell lines studied, the number EV-sorting domains (encapsulation index above one) ranged from 30 to 37 (FIG. 5B). A total of 24 domains were conserved, indicating robust EV-sorting ability across different cellular contexts. The relative performance of each domain in the conserved subset of each cell line was determined on the basis of intravesicular Tluc activity (FIG. 5C). On average, TSPAN2, CD63 and TSPAN3 demonstrated the highest EV-sorting ability in a reproducible manner.

The present inventors then consulted online databases to gain insight on the potential mechanism underlying the exceptional EV-sorting ability of this conserved subset. According to the annotation available on UniProtKB, most domains were featured by four-pass transmembrane structure except for the three calmodulin proteins and ANXA11 (FIG. 5D). Molecular interactions derived from the STRING database were also graphed (FIG. 5E), revealing central roles of CD9, CD63, and CD81 which likely reflects their extensive previous use for EV engineering. Interestingly, calmodulin proteins seem to operate irrespective of the rest, while there exists weak evidence for the interaction of TSPAN2 and TSPAN3 with CD63 due to that fact that TSPAN2 and TSPAN3 have previously never been regarded as EV proteins and thus never used or considered for use as scaffold proteins for loading POIs into EVs.

### Example 6—TSPAN2 and TSPAN3 as Potential EV-Sorting Domains-Comparison with CD63

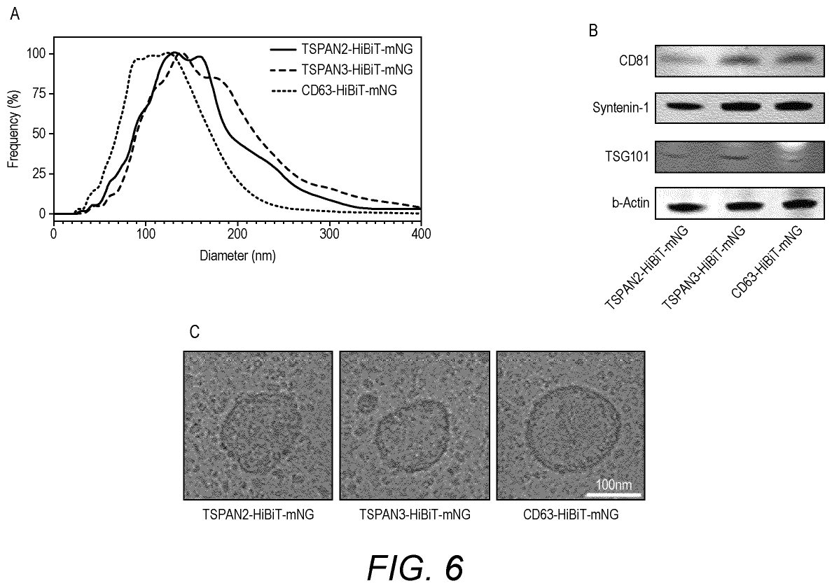

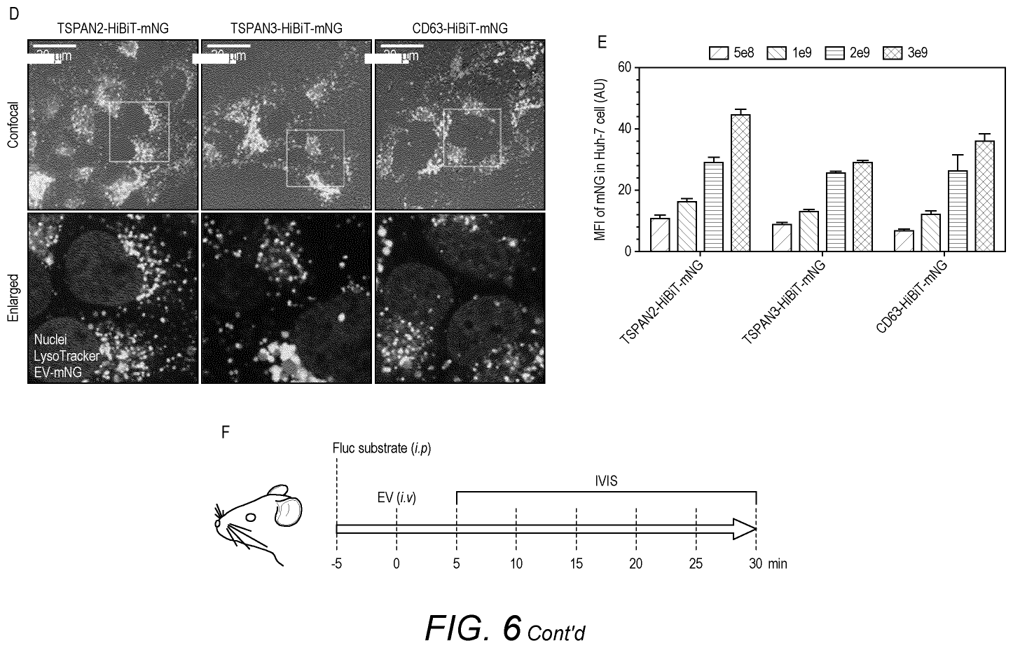

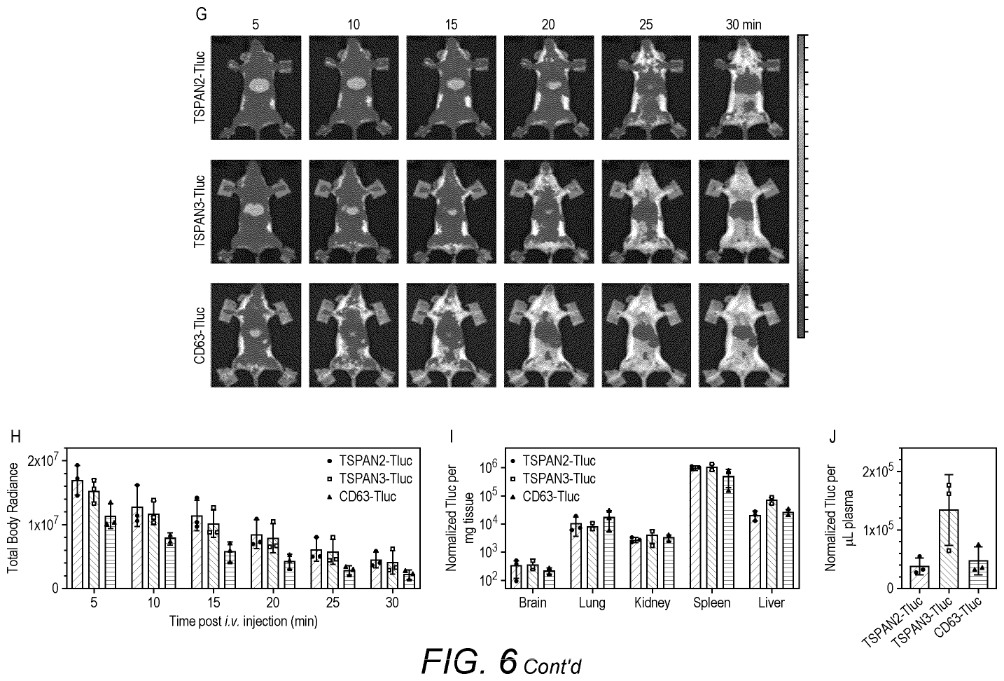

TSPAN2 and TSPAN3 stood out as promising candidates for sorting protein-of-interest into small EVs from both the screen and validation results. Further characterization of both types of engineered EVs revealed a narrow size distribution with a medium hydrodynamic diameter of approximately 120 nm (FIG. 6A). Common EV markers, like CD81, syntenin-1 and TSG101, were found in these EV preparations (FIG. 6B). As determined by cryo-electron microscopy, the morphological appearances of TSPAN2- and TSPAN3-engineered EVs were typical for EV features, like membrane structure and size (FIG. 6C).

To explore whether TSPAN2 and TSPAN3 confer distinct biological properties on the engineered EVs in comparison to CD63, the present inventors leveraged the mNG and Tluc reporter to examine cellular uptake and in vivo distribution, respectively. As depicted in the confocal microscopy images, all types of engineered EVs were efficiently internalized by Huh-7 cells and largely trafficked to lysosomes (FIG. 6D). Similar trends in terms of cellular uptake were observed by titrating the EV dose (FIG. 6E; Figure S6). Due to the swift distribution and catabolism of EVs in-vivo, the luciferin substrate was intraperitoneally dosed 5 min before intravenous administration of engineered EVs and Tluc activity in the mouse was monitored serially through IVIS spectrum in-vivo imaging system (FIG. 6F). For all three types of EVs, we observed rapid distribution to liver and spleen and a steady decline of whole-body activity over 30 min (FIG. 6G). Interestingly, TSPAN2 and TSPAN3-engineered EVs exhibited higher retention in the timeframe acquired (FIG. 6H). Tluc activity in major organs ex vivo and plasma were also measured, the results of which supported their dominant hepatic and splenic accumulation, and most importantly, improved exposure compared to CD63 (FIG. 6I; FIG. 6J).

In detail, FIG. 6 shows the results of characterization of TSPAN2 and TSPAN3-engineered EVs. (6A) Size distribution of engineered EVs as measured by NTA. (6B) Western blot of engineered EVs. (6C) Cryo-electron microscope images of engineered EV. (6D) Confocal microscope pictures of Huh-7 cells treated with mNG labeled-EVs for 4 hr. (6E) Mean fluorescence intensity of Huh-7 cells treated with mNG labeled-EVs for 8 hr. N=3. (6F) Scheme for examining the in vivo distribution of Tluc-labeled EVs. The mouse was immediately sacrificed after completion of IVIS session to collect major organs and plasma. N=3. (6G) Representative IVIS images of mice injected with Tluc labeled-EVs. (6H) Quantification of total body radiance of mice injected with Tluc labeled-EVs. (6I) Quantification of Tluc labeled-EVs in organs ex vivo. (6J) Quantification of Tluc labeled-EVs in plasma.

In summary, two mammalian proteins, TSPAN2 and TSPAN3, were for the first time surprisingly discovered by the present inventors to be superior to the benchmark domains CD63, CD9 and CD81 etc. It is known that certain domains have excellent EV-sorting ability in specific cell line(s) but not others (as shown in FIG. 5B). For instance, the viral glycoprotein gag operated impressively in HEK-293T, Freestyle 293-F, Huh-7, and to a less extent, in TCMK-1 cells, but rather poorly in MSC. The present inventors have identified as many as 24 domains that retained EV-sorting ability in a wide range of cell lines tested. Furthermore, the overall weak internal molecular interaction for the conserved subset is indicative of heterogeneous mechanisms of biogenesis of engineered EVs and subsequently distinct molecular fingerprints as well as biological properties, this also highlights that the discovery of these particular scaffold proteins was unpredictable and only as a result of a highly inventive and large scale screening effort. The results of the screen and validation experiments highlighted the exceptional EV-sorting ability of TSPAN2 and TSPAN3. Their splice isoforms were also explored but showed barely any EV-sorting ability (data not shown). In addition, TSPAN2- or TSPAN3-engineered EVs were efficiently taken up by cells in vitro and even more bioavailable over time following intravenous administration in mouse than the benchmark CD63.

### Example 7: Initial Results of Large Scale Functional Screening to Identify EV Novel EV Protein Candidates Capable of Allowing Co-Expression of Multiple Constructs

Following the experiments detailed in Examples 1-6 further investigation into the delivery of functional cargos were conducted. Approximately 280 potential EV proteins were screened in total. Importantly scaffold/carrier EV proteins were not merely screened for their presence/prevalence on exosomes but they were tested for functional delivery of a reporter protein via two different systems (a co-culture system and a method of adding purified EVs direct to cells). These tests were also run in a large range of different cell lines to ensure the findings were robust and not cell line specific.

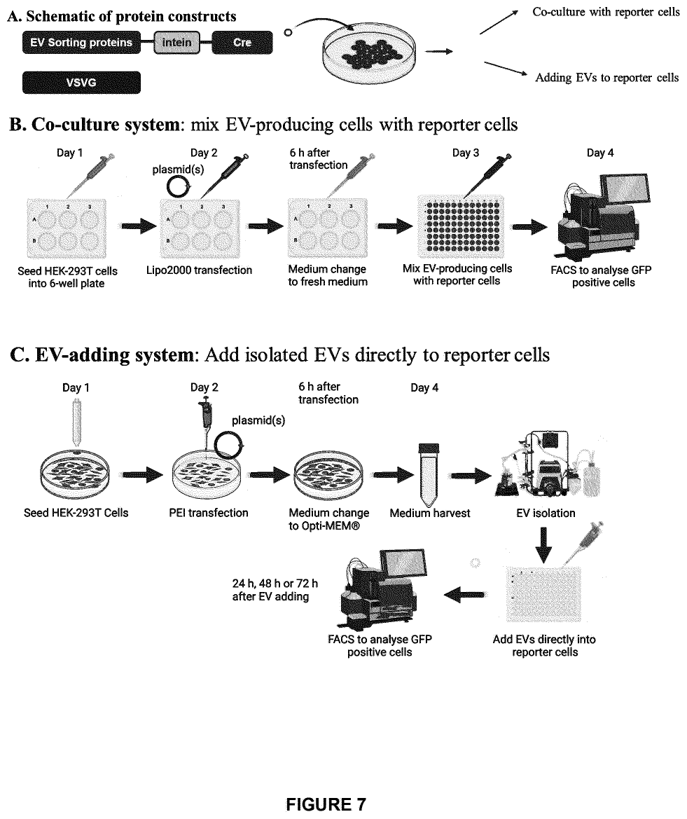

FIG. 7A shows a schematic of the Cre reporter protein used to identify promising EV protein candidates. FIG. 7B depicts the Co-culture method. In detail this method involves mixing EV producing cells with reporter cells, allowing the EVs to be taken up by the cells and on day 4 measuring by FACS to analyze GFP positive cells. FIG. 7C depicts the EV adding system. In more detail this method involves isolation and purification of the EVs before they are added to the reporter cells and then analyzed by FACS to detect GFP positive cells. These two different systems were employed in combination because the co-culture system is highly economical, fast, and easy to operate and suitable for preliminary screening and the adding system provides a second more accurate but time-consuming screen.

HEK-293T cell were used as the EV producing cells. The HEK293T cells were co-transfected with the EV-sorting protein-Intein-Cre plasmid and VSV-G plasmid. In both the co-culture and EV adding system the following reporter cell lines were tested: Hela TL; T47D TL and B16F10 TL. These reporter cells respond using the traffic light reporter system to the recombination by the Cre cargo delivered by the EVs. It is therefore possible to detect the delivery of functional Cre by the EVs by the colour change observed. In the co-culture system (FIG. 7B) the EV-producing cells and reporter cells are mixed, 1:1 and 1:5 ratios (producing cells: reporter cells. In the EV adding system EVs were added directly to reporter cells at 1E10, 1E9 and 1E8 doses.

The results of this follow-up functional screen are given in Examples 7-12 below. Materials and methods for Examples 7-12 as follows:

### Constructs Generation

Codon-optimized DNA sequences coding for the scaffold protein including tetraspanins (2-33), late endosomal proteins (STR3N, LAP4B, STAR3, SPP2A, SNX14, NTRK1), cytoplasmic proteins (syntenin1, ANXA4, CALM2, ANX11, CALM3, CALM1, GDIB), single-Pass Transmembrane Proteins (CD316, ICAM1, PTGFRN, Lamp2B), membrane associated proteins (ARRDC1, Basp1, Myr) and other proteins (GAG, CLD1, AAAT) were ordered from TB (Twist Bioscience, US). Tetraspanin-1, intein-cre and VSV-G were ordered from IDT (Integrated DNA Technologies, USA). The scaffold proteins were first cloned into pLEX vector and then the intein-cre was insert into plex-scaffold plasmids by using according restriction endonuclease site.

### Cell Culture

In this study, HEK-293T cells were employed to generate functional EVs, and they were cultured in DMEM medium (high glucose) supplemented with 10% fetal bovine serum (FBS, Gibco, USA) and 1% anti-antibody (Gibco, USA). Cells were cultured at 37° C. in a humidified air atmosphere with 5% CO2. The reporter cell lines (Hela-TL, T47D-TL, MSC-TL, and B16F10-TL) were cultured in the same medium and conditions as the HEK-293T cells. THP1-TL, RAW246.7-TL, K562-TL were cultured with RPMI-1640 medium supplemented with 10% fetal bovine serum (FBS, Gibco, USA) and 1% Anti-anti (Gibco, USA).

### Ev Production

The EVs were produced by transient transfection of the transgenes by using polyethylenimine (PEI). To be specific, the HEK-293T cells were seeded into 15-cm dishes with 10 million cells/dish with full DMEM medium. The day after, the cells were transfected by the transgenes and the medium was changed to Opti-MEM medium (Gibco, USA) with 1% Anti-anti 6 h after transfection. After 48 h of transfection, the conditional medium (CM) was collected and went through centrifugation (700 g, 5 min followed by 2000 g, 10 min). The supernatant was then filtered by 0.22 μm filter system.

### Ev Isolation

Tangential flow filtration (TFF, MicroKross, 20 cm², Spectrum labs) was used to isolate EVs from the filtered CM. The cutoff of TFF is 300 kDa and the particles bigger than 300 kDa are remained in the system and concentrated. And then the concentrated particles were further concentrated by Amicon Ultra-15 100 kDa (Millipore) spin filter, centrifuged at 4000×g for 30 min to several hours at 4° C. depending on the amount EVs in the samples. At last, the concentrated EVs were collected in maxirecovery 1.5 ml Eppendorf tubes (Axygene, USA) and the concentrations were detected by Nanoparticle Tracking Analysis (NTA).

### Nanoparticle Tracking Analysis (NTA).

The EV samples were diluted with freshly 0.22 μm-filtered PBS before checking the particle sizes and concentrations of the samples with a NanoSight NS500 instrument. 5 videos with time longer than 30 seconds were taken at the cameral level of 15 in light scatter mode. The obtained data were analysed by the equipped NTA 2.3 software and all the samples were analysed with the same constant setting.

### Macsquant Flow Cytometry

After the different traffic-light reporter cells were added into EVs at different time points, or after the reporter cells were co-cultured with EV-producing cells for 24 h, they were checked for GFP expression by suing MACSQuant flow cytometry (Miltenyi biotec, Germany). In brief, the cells in 96-well plates were washed by PBS one time after discarding supernatant and then the cells were trpsinized for 5 min at 37° C. And then the cells were resuspended by cell medium supplemented with 10% FBS. After adding DAPI to check the cell viability, the cells were sampled by MACSQuant with the same setting for all the measurements of one specific reporter cell line. At last, the data were analysed by flowjo to calculate the percentage of GFP positive cells.

### Ev Uptake in Reporter Cells

The reporter cells were seeded into 96-well plates one day before adding the engineered EVs. Forty-eight hours later the percentage of GFP positive cells were measured by MACSQuant.

Co-Culture of EV-Producing Cells with Reporter Cells

HEK-293T cells were seeded into 6-well plate with 0.5 million cells per well. The next day when the cells reached 60-70% confluence, corresponding constructs were transfected into the wells by using lipofectamine2000 (Invitrogen, USA) according to the protocols provided by the manufacturer. 6 h after transfection, the medium was changed to fresh medium (DMEM+10% FBS+1% Anti-anti) to reduce the toxicity of lipofectamine2000 used. 24 h after plasmid transfection, the cells were trypsinized and counted and mixed with the corresponding reporter cells with the ratios 1:1 or 1:5 (ratio=EV-producing cells: reporter cells) in 96-well plate. After co-culturing for 24 h, the cells were trypsinized and measured by MACSQuant to check the percentage of GFP positive cells.

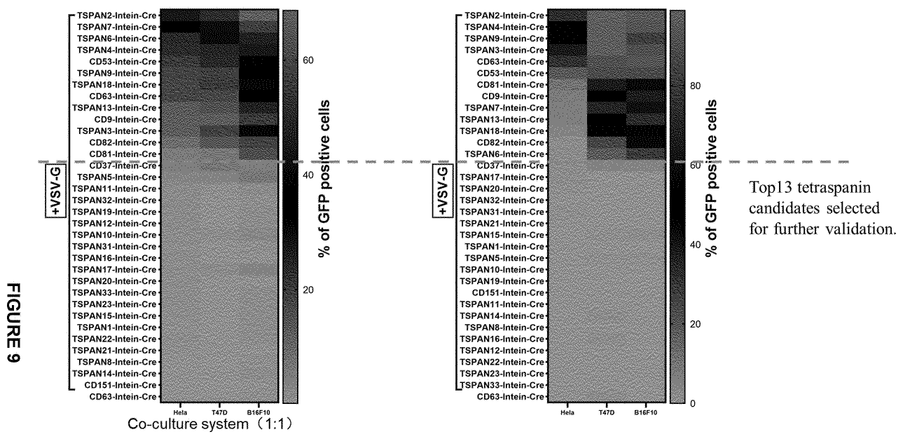

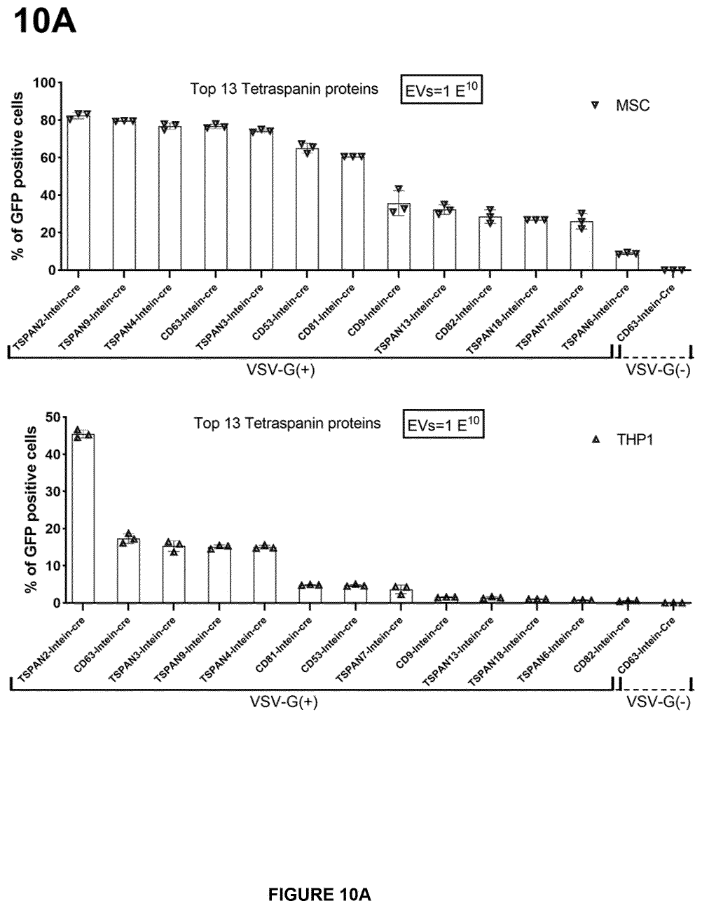

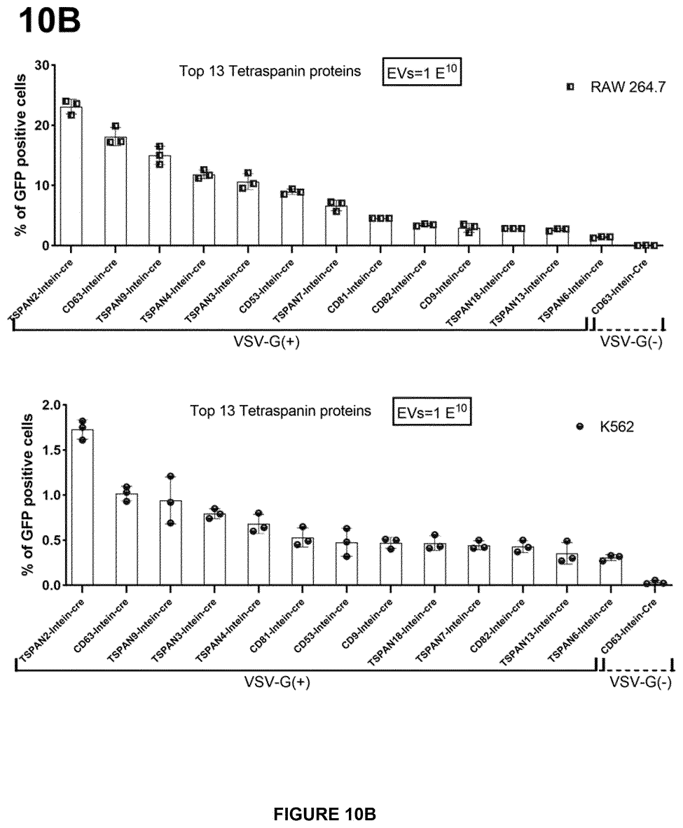

Different categories of possible EV scaffold/carrier protein were tested including cytoplasmic, late endosomal, single pass transmembrane proteins and tetraspanins. Of the 280 proteins screened the tetraspanins showed the most promise. 33 tetraspanins were tested (see FIG. 9), the top 13 of which were then taken forward after initial screen for further analysis (see FIGS. 9 and 10).

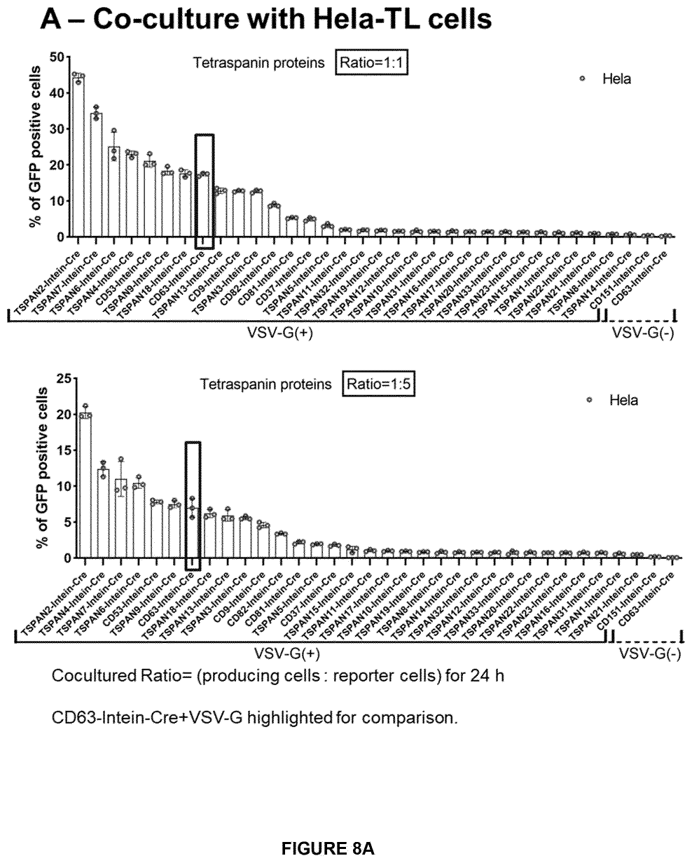

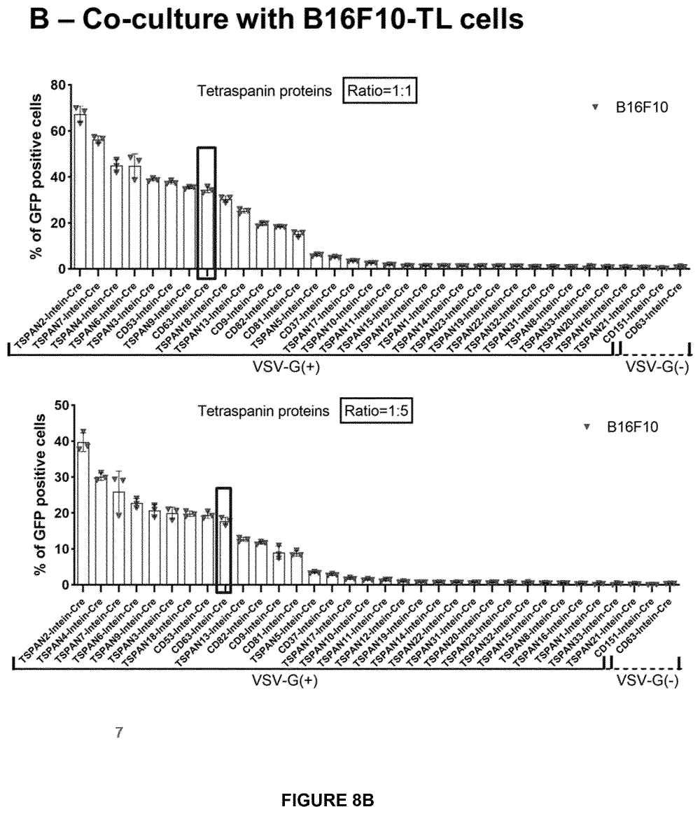

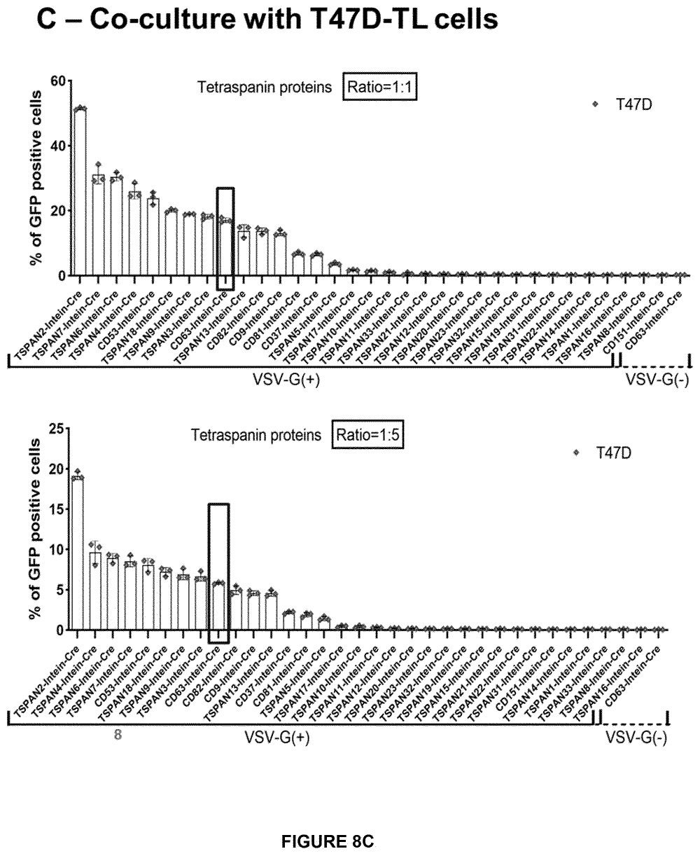

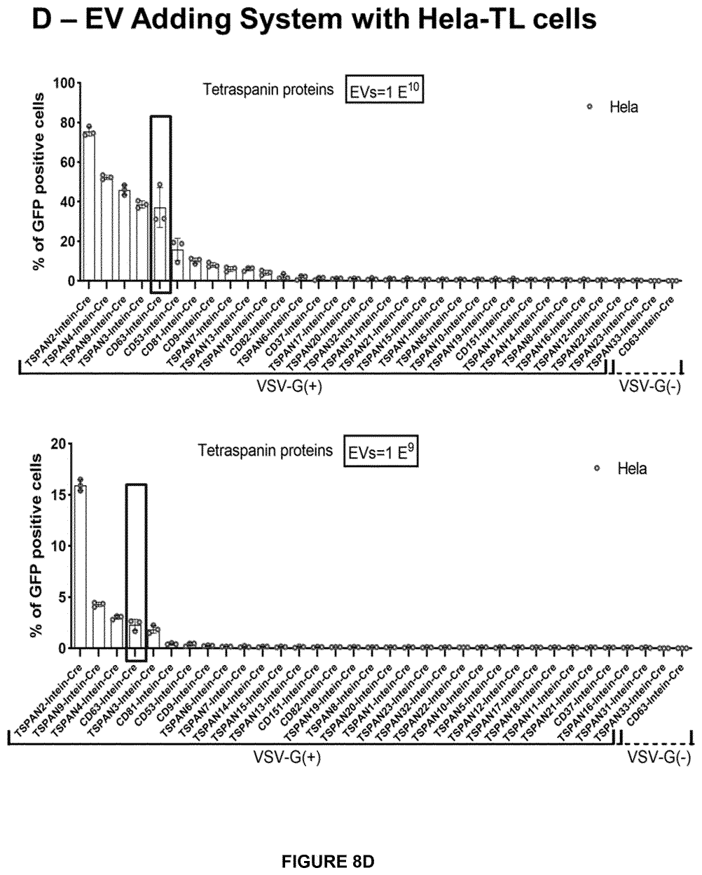

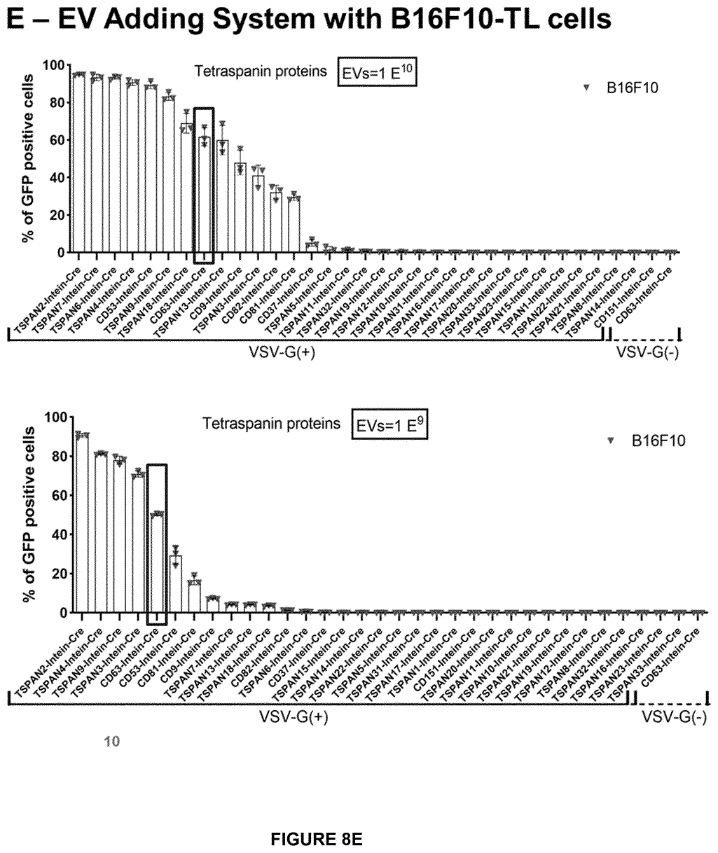

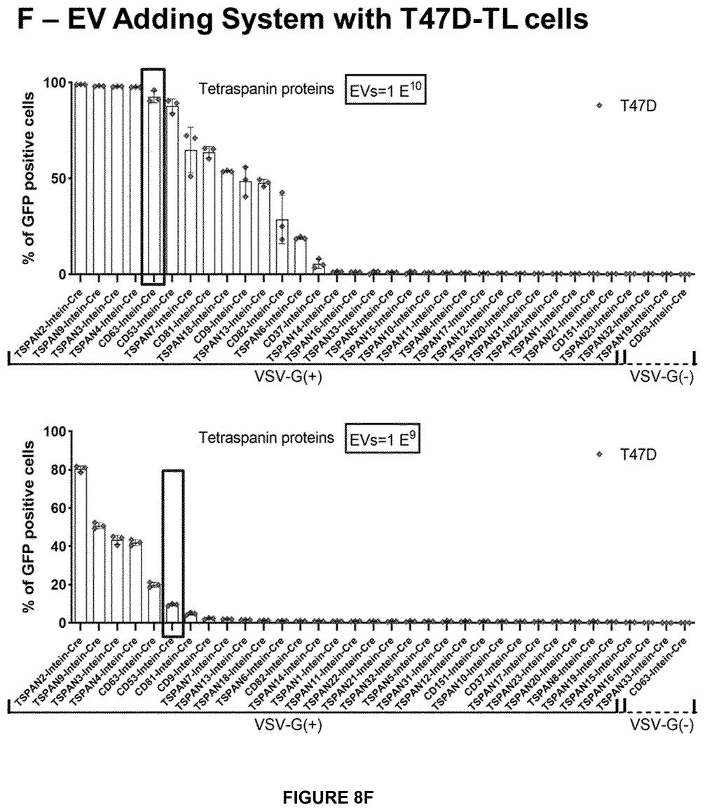

As can be seen from FIGS. 8A-F by far the most efficient tetraspanin EV protein for co-delivery of VSVG and Cre was TSPAN2 (the results for the classical EV protein CD63 are highlighted for comparison). This was true in every cell line and by both test systems. This further confirmed the evidence from Examples 1-6 which identified TSPAN2 as a novel and highly effective EV scaffold protein which is able to transport cargos into EVs across a wide range of different EV cell sources.

FIG. 9 shows the same data but depicted as a heat map of % of GFP positive cells. FIG. 9 also shows the cut off point for the top 13 tetraspanin candidates selected for further validation. TSPAN2 performed the best across all cell lines tested.

### Example 8-Further Validation of Top 13 Tetraspanin Candidates Using Other Hard to Transfect Cell Lines

The top 13 candidates as identified in FIG. 9 were then tested further in different, harder to transfect cell lines: MSC, THP1, Raw264.7 and K562 cells. The EV adding system, as described above and depicted in FIG. 7C was used for this experiment.

As can be seen from FIG. 10, once again TSPAN2 is still the best candidate among the top 13 scaffold protein candidates tested in hard to transfect cell lines. This demonstrates the broad utility of TSPAN2 as a scaffold protein.

### Example 9-Comparison of TSPAN2 with Classical EV Scaffold Proteins (CD9/63/81)

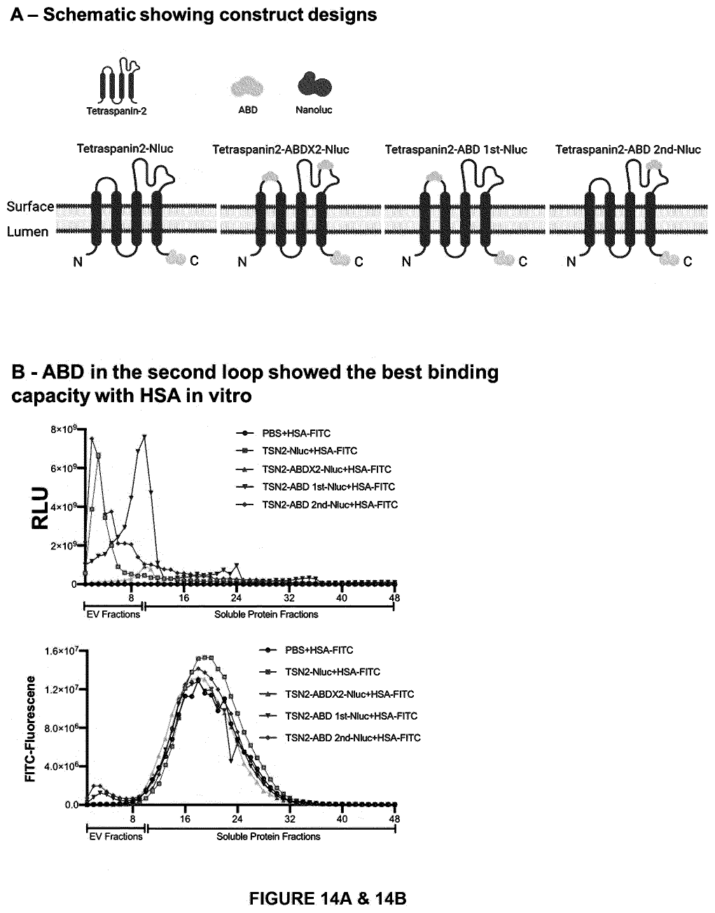

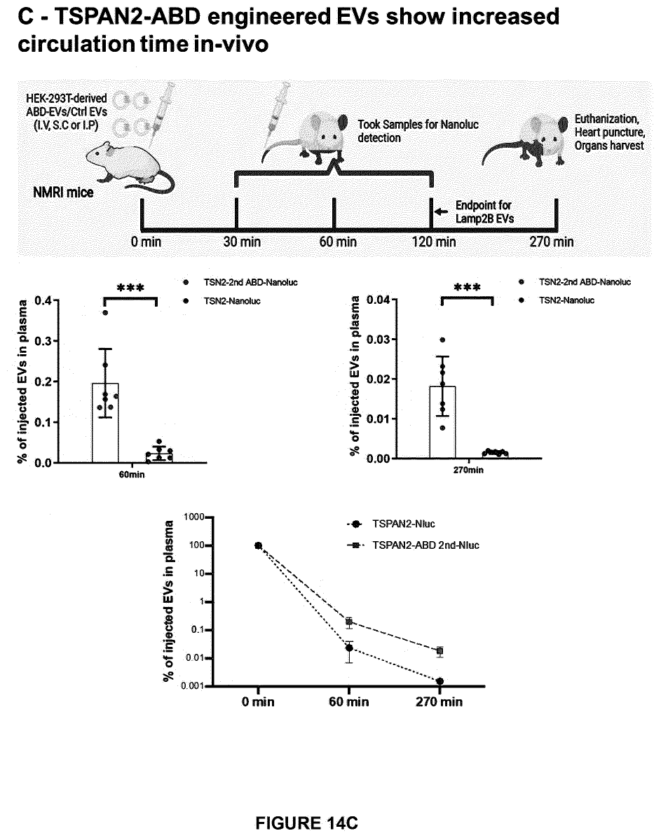

The present inventors then wished to compare the efficacy of the newly identified EV protein candidate, TSPAN2, against classical EV proteins. The co-culture method described above was utilized to test the ability of TSPAN2 vs the classical EV proteins to allow co-expression of multiple constructs on the same vesicles. This experiment was conducted using 3 cell lines, Hela, T47D and B16F10 and compared the ability of TSPAN2 to act as a scaffold protein to three classical EV proteins: CD9, CD63 and CD81. In this experiment the cargo protein is albumin binding domain (ABD) plus NanoLuc. as a reporter (FIG. 14 shows the ABD construct designs).

FIG. 11 indicates that albumin binding domain (ABD) in the second loop of traditional tetraspanins (CD9, CD63 and CD81) counter-acted the VSV-G delivery effect while ABD in the second loop of TSPAN2 enhanced the VSV-G delivery effect. This shows that surprisingly TSPAN2 is superior to its paralogue CD9 and other classical EV proteins CD63 and CD81 when it comes to both allowing genetic manipulation for the purposes of carrying a POI and for enabling co-localization of multiple POIs on the same EV. The present inventors have discovered that unlike the “classical” EV-associated tetraspanins (CD9, CD81 and CD63), TSPAN2 apears to be capable of being engineered whilst maintaining co-localisation with another construct, in this case VSV-G-Intein-Cre. This enables TSPAN2 to function as a much more flexible tool for EV engineering allowing much simpler development of “platform technologies” as compared to previous scaffold proteins.

### Example 10-Confirmation that Lack of Colocalization Causes Reduced POI Delivery

The present inventors then went on to confirm by FACs that the reduced POI delivery seen in Example 9 and FIG. 11 is due to lack of co-localization of the two necessary constructs. Details of FACs protocol as above.

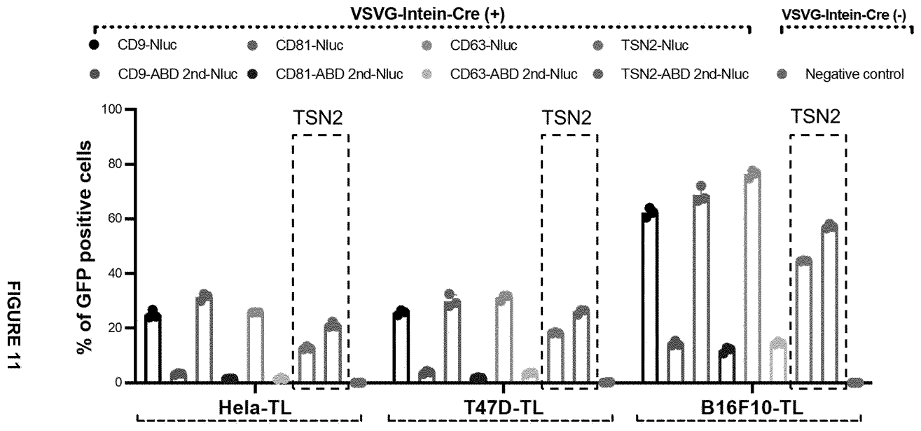

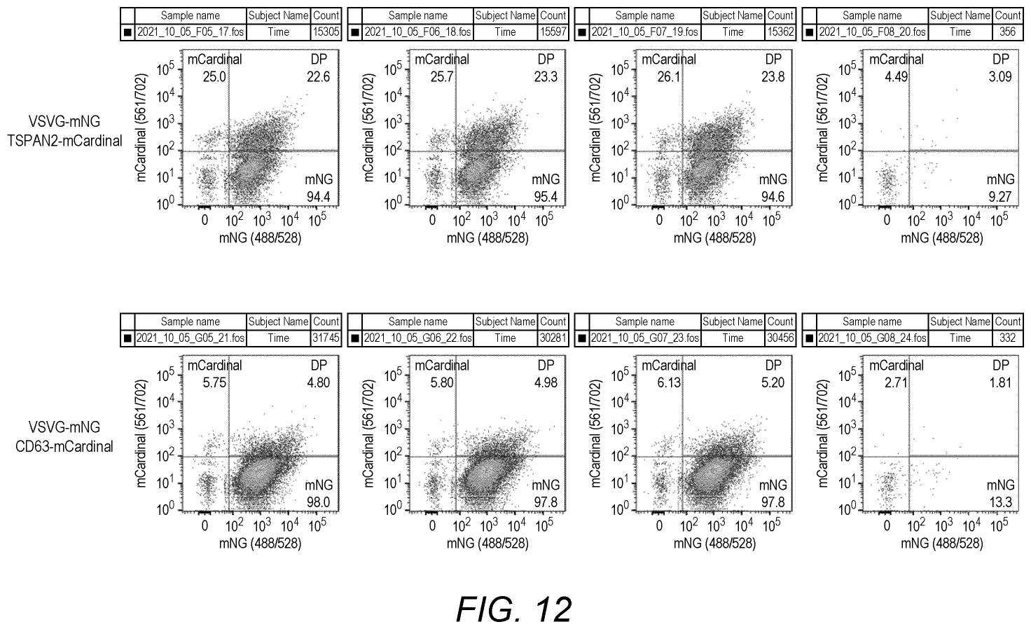

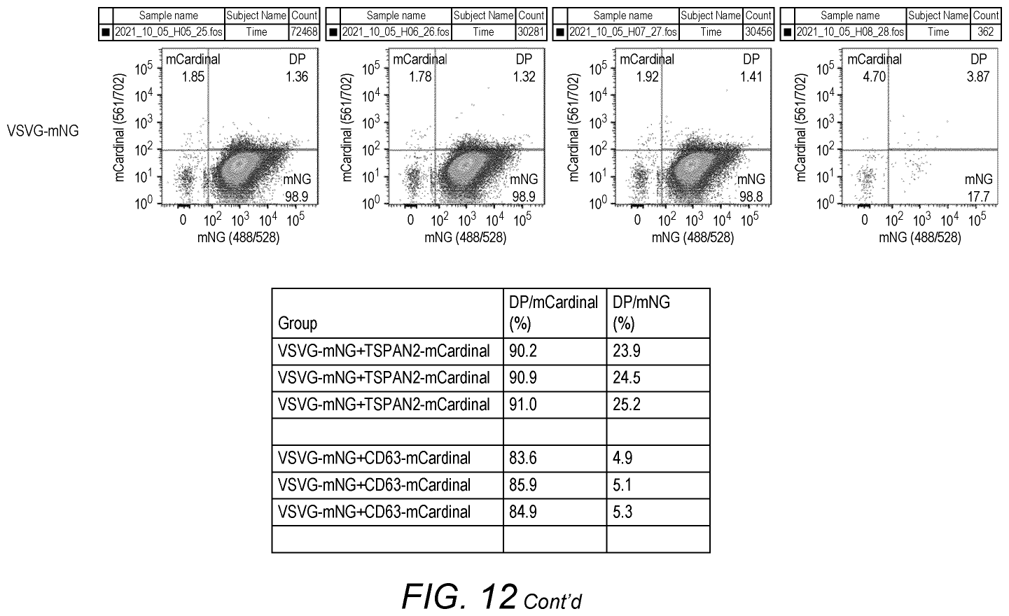

FIG. 12 shows the results of FACS analysis comparing two fusion protein constructs (i) VSVG-mNG+TSPAN2-mCardinal and (ii) VSVG-mNG+CD63-mCardinal. FIG. 12 shows that whilst mCardinal values are fairly similar between TSPAN2 and CD63 it can be seen that the mNG values (corresponding to the co-localized VSVG levels) are significantly lower when CD63 is over expressed as compared to when TSPAN2 is the scaffold used. The present inventors believe the variation in results between CD63 and TSPAN2 expressing EVs is due to the fact that these proteins localise to different populations of EVs which enable TSPAN2 to co-localize with a second construct much more readily than for CD63 to do so. This shows the difficulty in introducing multiple constructs into the exosomes when using the CD63 scaffold for engineering the POI. As shown in FIG. 11 engineering CD63 causes lower expression of other constructs and that TSPAN2 is therefore much better at loading cargos into EVs than CD63. It should also be noted that this data also indicates TSPAN2 can deliver luminal and surface display POIs at the same time.

### Example 11—In-Vivo Data Showing Functional Delivery of Cre Protein

The present inventors then went on to test the ability of TSPAN2 to delivery functional POIs in vivo. In order to test in-vivo delivery an intra-tumoral mouse model was employed.

C57BL/6 mice were ordered at around 5-week age with 20 g body weight. The animals were acclimated to their new surroundings at least one week before the experiment. B16F10-TL cells were harvested and resuspended in PBS and then inoculated with the cells subcutaneously at the number of 0.5 million per mouse. After 10 days of inoculation, when obvious tumors were formed, engineered EVs were injected directly into the tumors. The injected volume was 50 μL per mouse with 7.5×10¹⁰ EVs. 4 days after intra-tumor injection of EVs, the mice were sacrificed and tumors were harvested. Half of the tumor tissues were fixed with PFA and sent for slides making and the other half were immersed in lysis buffer (0.1% TritonX-100) for DNA isolation. When the slides with tumoral tissue on were ready, IHC staining for GFP expression were performed and the tissues in lysis buffer were homogenised with tissue lyzer machine. 50 μL of the tissue lysate were taken for DNA isolation by using the Maxwell® RSC tissue DNA Kit (Promega, USA).

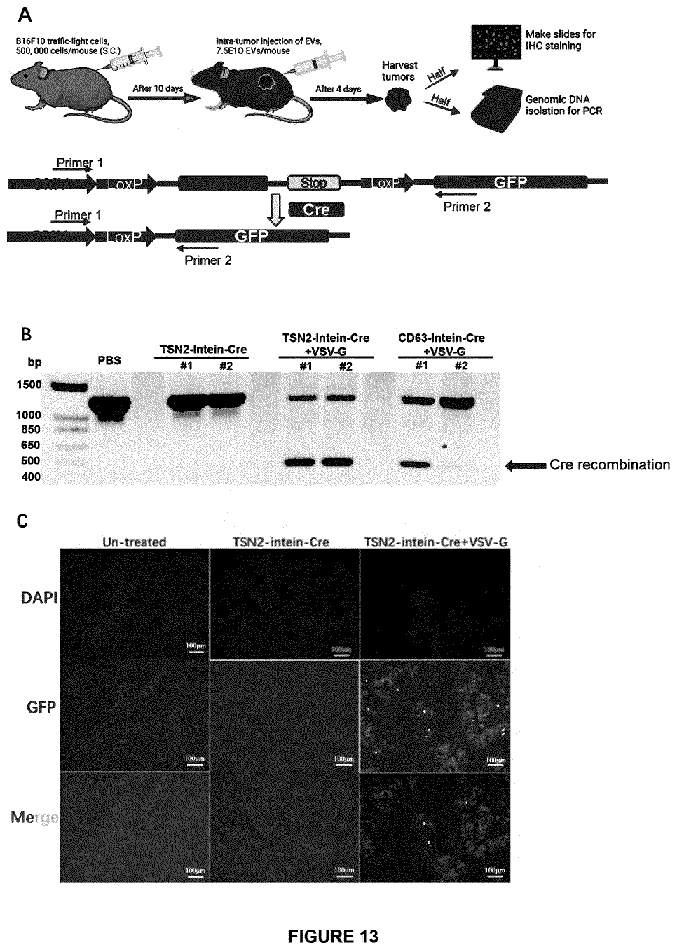

FIG. 13 A shows a schematic of the intra-tumor injection of melanoma model and primer design. FIG. 13 B shows the PCR results demonstrating effective Cre recombination in-vivo. The band intensity in FIG. 13B shows that TSN2-engineered EVs display better in-vivo recombination than CD63-engineered EVs. FIG. 13 C shows microscopy again demonstrating functional delivery of Cre proteins by TSPAN2 engineered EVs in melanoma models. FIG. 13 confirms that TSPAN2 is more effective than CD63 at delivery of functional cargos in-vivo.

### Example 12—Use of TSN2 to Display ABD as Alternative POI

Next the present inventors tested the ability of TSPAN2 to express albumin binding domain (ABD) on EVs. ABD is used to attract albumin to the EV and thus extend the half-life of the EV when in circulation. FIG. 14A shows schematics for the TSPAN2 constructs used in this experiment. ABD is displayed on the surface of the EV.

The binding of the ABD-engineered EVs was evaluated with commercially available FITC-labelled human serum albumin (HSA-FITC). The isolated EVs were incubated with HSA-FITC and then size-exclusions by QEV were performed. 48 fractions were collected from each sample, with 300 μl per fraction. Therefore, the 1 to 9 fractions were supposed to be the part that contained only EVs (EV fractions) while the 10 to 48 fractions contained mainly the soluble proteins. The results are shown in FIG. 14B showing that ABD expressed in the second loop of TSPAN2 results in the best HSA binding.

FIG. 14 shows the in-vivo data demonstrating that EVs decorated with albumin by displaying ABD on the surface of EVs fused to TSPAN2 have an extended circulation time in-vivo. Previously using CD63 as the scaffold protein half-life extensions of around 10 fold have been observed. Here using TSPAN2 the half-life extension is increased beyond that capable with CD63 to 12 fold.

This shows that TSPAN2 is an effective scaffold for a binding protein as the POI and furthermore again demonstrates that delivery of a POI by TSPAN2, in this case an albumin binding domain, achieves excellent results superior to those seen using classical EV proteins. This would not have been expected without the major screening effort by the present inventors. Prior to development of the complex screening method described above TSPAN2 had never been considered as a viable scaffold protein due to it not being highly enriched in its native form on EVs.

### Example 13—TSPAN2 Delivery of Ago2 Ribonucleic Acid Complex

The present inventors next tested the ability of TSPAN2 to deliver a ribonuclear protein complex as the POI. To do this a model of EV-mediated silencing of Gapdh on mRNA and protein levels was used. shRNA against Gapdh (shGapdh) was indirectly loaded into EVs by fusing RNA-binding protein Argonaute 2 (AGO2) to EV-sorting domains MYR and Tetraspanin 2 (TSN2). Additionally, VSVG was used as a fusogenic protein to ensure cargo delivery to the cytosol of recipient cells. Constructs were transiently co-transfected into parental HEK293T cells to produce engineered EVs, which were then isolated and purified. Upon addition to recipient cells, EVs achieved dose-dependent target knock-down on both mRNA and protein levels after 48 hours. Enhanced silencing effects were observed when shGapdh was enriched in EVs by AGO2 fused to EV-sorting domains.

### Cell Culture

Cells were cultured in complete growth medium consisting of Dulbecco's Modified Eagle's Medium High Glucose (DMEM, Gibco Thermo Fisher Scientific), supplemented with 10% Fetal Bovine Serum (FBS, Gibco, Thermo Fisher Scientific) and 1% Antibiotic-Antimycotic (AA, Gibco, Thermo Fisher Scientific) and maintained at 37° C., 5% CO2 atmosphere unless otherwise specified.

For EV production, 1×10⁷ human embryonic kidney cells (HEK293T) were seeded in 15 cm culture dishes and after 24 h, were transfected with plasmids of interest complexed with branched polyethylenimine (Sigma-408727; 30 μg DNA: 45 μg PEI) in OptiMEM (Gibco, Thermo Fisher Scientific). 4 h after transfection, medium was changed to OptiMEM reduced serum medium supplemented with 1% AA. Cells were incubated for 48 h before EV isolation.

For EV uptake studies, mouse neuroblast (Neuro-2a) cells were seeded in 96-well plates (1×10⁴ cells/well) or 24-well plates (5×10⁴ cells/well) for RNA and protein extraction, respectively. After 24 h, EVs were added (5×10⁷-5×10⁹ EVs/well of 24-well plate; 1×10⁷-1×10⁹ EVs/well of 96-well plate) and cells were incubated for another 48 h before RNA or protein extraction.

### Extracellular Vesicle Isolation

Conditioned medium (CM) from previously transfected HEK293T cells was collected and spun at 700×g for 5 min and subsequently at 2000×g for 10 min to remove cell debris and larger particles. The supernatant was passed through a 0.22 μm vacuum filter and concentrated by ultrafiltration (UF) with Amicon Ultra-15 100 kDa molecular weight cut-off spin-filter (Millipore) to a final volume of 500 L and later loaded into qEVoriginal/70 nm Legacy size exclusion column (Izon Science). According to the manufacturer's instruction, the vesicular fractions were collected and further concentrated with Amicon Ultra-2 10 kDa molecular weight cut-off spin-filter (Millipore) to a final volume of 100 μl and stored at −80° C. for further analysis.

### Nanoparticle Tracking Analysis

Particle size and concentration of the samples were determined via nanoparticle tracking analysis (NTA) using NanoSight NS500 equipped with NTA 2.3 analytical software and a 488 nm laser. Briefly, samples were diluted in 0.22 μm filtered PBS and for each sample five 30-s videos were recorded and analysed.

### Rna Isolation and RT-qPCR

Neuro-2a cells that were previously incubated with EVs in 96-well plates were detached with Trypsin-EDTA (0.25%, Gibco, Thermo Fisher Scientific), transferred to v-bottom 96-well plates and spun at 900×g for 5 min. Optionally, cell pellets were stored at −80° C. until further processing. RNA was extracted with Maxwell RSC simplyRNA Cells Kit (Promega) according to manufacturer's instructions on Maxwell RSC Instrument (Promega). Resulting RNA concentrations were determined using Qubit RNA High Sensitivity Assay (Invitrogen, Thermo Fisher Scientific) on Qubit 3 Fluorometer (Invitrogen, Thermo Fisher Scientific). 100 ng RNA were then reverse transcribed in accordance with High Capacity cDNA Reverse Transcription Kit (Applied Biosystems, Thermo Fisher Scientific) instructions. An equivalent of 5 ng RNA was used per reaction of qPCR with TaqMan Fast Advanced Master Mix (Applied Biosystems, Thermo Fisher Scientific) on CFX96 Touch Real-Time PCR Detection System (Bio-Rad) with the following primers and probes (Gapdh: forward GCC TTC CGT GTT CCT ACC, reverse CCT CAG TGT AGC CCA AGA TG, probe/5HEX/CGC CTG GAG/ZEN/AAA CCT GCC AAG TA/3IABKFQ/; Hprt: forward GCC CTC TGT GTG CTC AAG, reverse CCC CGT TGA CTG ATC ATT ACA, probe/56-FAM/AGC AGG TCA/ZEN/GCA AAG AAC TTA TAG CCC/3IABKFQ/).

### Protein Extraction and Western Blot

Neuro-2a cells that were previously incubated with EVs in 24-well plates were detached with Trypsin-EDTA (0.25%, Gibco, Thermo Fisher Scientific), transferred to tubes and spun at 900×g for 5 min. Optionally, cell pellets were stored at −80° C. until further processing or lysed in RIPA buffer. Cell debris was pelleted at 13000×g for 12 min and supernatant was transferred to a new tube. Subsequent steps were performed according to NuPAGE (Invitrogen, Thermo Fisher Scientific) technical guide using NuPAGE® Novex® 4-12% Bis-Tris Protein Gel (Invitrogen, Thermo Fisher Scientific) run at 120 V in NuPAGER MES SDS running buffer (Invitrogen, Thermo Fisher Scientific) for 2 h. The proteins on the gel were transferred to a nitrocellulose membrane (iBlot 2 Transfer Stacks, Invitrogen, Thermo Fisher Scientific) for 7 min using the iBlot 2 Gel Transfer Device (Invitrogen, Thermo Fisher Scientific). Membranes were blocked with Odyssey blocking buffer (LI-COR) for 60 min at RT with gentle shaking. After blocking, the membrane was incubated overnight at 4° C. or 1 h at RT with primary antibody solution (1:10,000 dilution for anti-β-actin [A5441, Sigma] and anti-Gapdh [PA1-16777, Invitrogen, Thermo Fisher Scientific]). The membrane was washed with PBS supplemented with 0.1% Tween 20 (PBS-T) for 5 min, 5 times and incubated with the corresponding secondary antibody (LI-COR) for 1 h at RT (1:15,000 anti-mouse 680LT to detect β-actin; 1:15,000 dilution anti-rabbit IRDye@800CW to detect Gapdh). Membranes were washed with PBS-T 5 times within 25 min, one time with PBS and visualized on the Odyssey infrared imaging system (LI-COR).

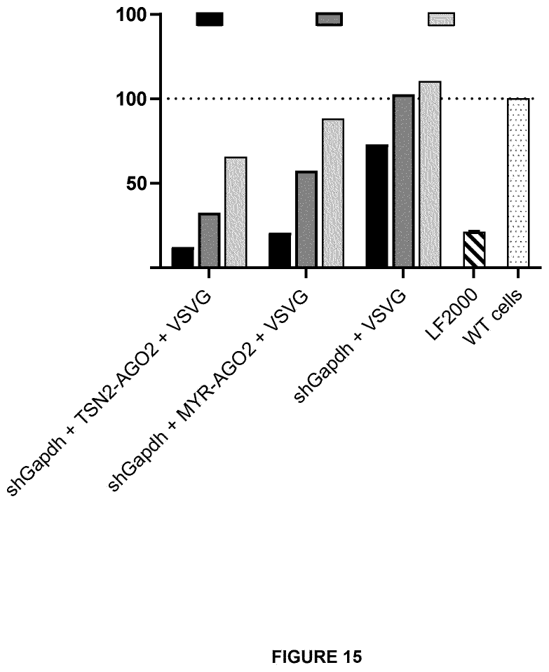

FIG. 15 shows the results of this experiment, showing that TSPAN2 can be used to deliver a ribonucleic acid cargos again displaying the broad utility of TSPAN2 as a scaffold protein for the delivery of a wide range of different types of cargo POIs.

### Example 14—TSPAN2 Delivery of mRNA

Following the highly promising results detailed above the present inventors also wished to test whether the TSPAN2 could be used to load mRNA via a nucleic acid binding protein.

PUFeng was selected as the nucleic acid binding protein and erythropoietin (EPO) designed to comprise an PUFeng binding site mRNA was utilised as the cargo nucleic acid. The following fusion constructs were compared to test their ability to load EPO mRNA into EVs:
TSPAN2-PUFeng
TSPAN2-intein-PUFeng
CD63-PUFeng
CD63-intein-PUFeng
TSPAN2-MS2 (no-bind control)
TSPAN2-intein-MS2 (no bind control)
CD63-MS2 (no bind control)
CD63-intein-MS2 (no bind control)

EV producing cells were cultured and transfected in 2 ml or 15 ml total volume in accordance with manufacturer's protocol with EV material being harvested after 72 h. To purify the harvested material cell cultures were spun down and then passed through 0.2 μm conical filter after which media was then added to Proteus X-spinner spin filters and spun at 2000×g for 30 minutes. RNA was extracted from the EVs using Maxwell RSC using ‘Maxwell® RSC miRNA from Plasma or Serum’ kit following manufacturer's instructions and quantified by nanodrop. RT-PCR was then conducted using High-Capacity cDNA Reverse Transcription Kit with oligo dT primers.

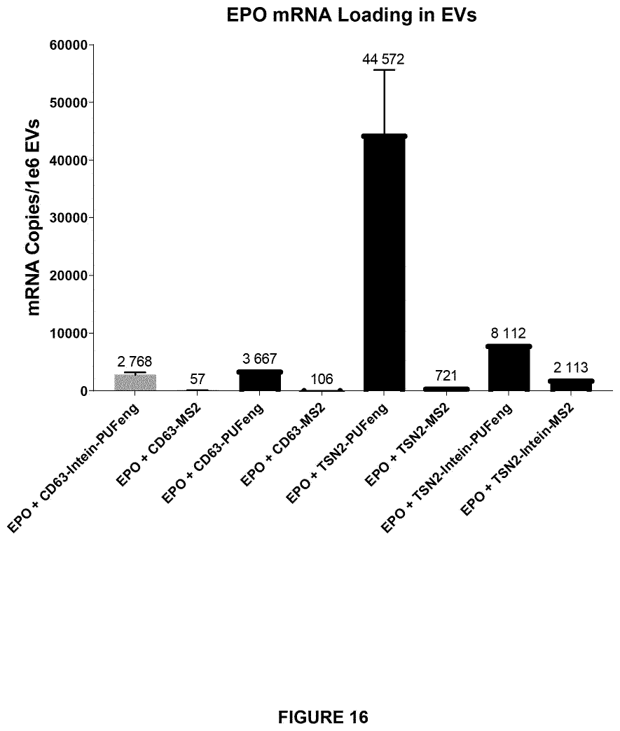

FIG. 16 demonstrates the more than 10 fold superior loading ability of TSPAN2 as compared to the classical EV scaffold protein CD63. When using TSPAN2 as the scaffold protein 44,572 copies of mRNA were loaded vs only 3,667 copies when CD63 was employed.

In detail FIG. 16 shows EPO mRNA loading into EVs was observed with a clear enrichment in PUFeng samples in comparison with MS2 (no-bind) controls, mRNA copy numbers in the producer cells remain similar (data not shown), indicating that the loading is an active process. Once again, this is a highly surprising discovery since TSPAN2 was previously not known to be highly expressed on EVs and therefor never previously considered as a promising scaffold protein especially not in the context of a nucleic acid cargo.

### Example 15—TSPAN2 and TSPAN3 Delivery of NanoLuc and msO×40L mRNA

The present inventors went on to investigate whether TSPAN2 and TSPAN3 could be used to load other mRNAs via the PUF nucleic acid binding protein. In these experiments, constructs comprising MS2, which is not an RNA binding domain, were used as a control.

NanoLuc mRNA and a codon optimised murine O×40L mRNA designed to comprise an PUFeng binding site mRNA were used as the cargo nucleic acids. The following fusion constructs were compared to test their ability to load NanoLuc or msO×40L mRNA into EVs:
TSPAN2-PUFeng
TSPAN2-MS2 (no-bind control)
TSPAN3-PUFeng
TSPAN3-MS2 (no-bind control)
CD63-PUFeng
CD63-MS2 (no-bind control)

HEK293T cells were cultured and transfected with the constructs by standard methods and the conditioned medium was collected from the cells 48 h post-transfection. EV material was purified by centrifugation at 700×g for 5 min to remove residual cells, then at 2000× g for 10 min to remove cell debris. The centrifuged medium was then filtered through a polyethersulfone (PES) membrane filter with 0.22 μm pore size (Techno Plastic Products). Filtered conditioned media were subjected to ultracentrifugation at 100,000×g for 90 min at 4° C. and the pellet was resuspended in 200 μl sterile PBS for immediate use.

RT-PCR was then conducted using High-Capacity cDNA Reverse Transcription Kit® (Thermo Fisher Scientific) with oligo dT primers.

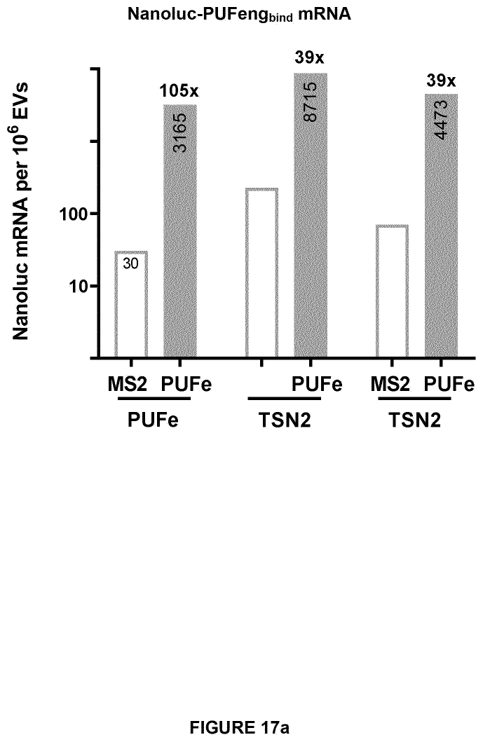

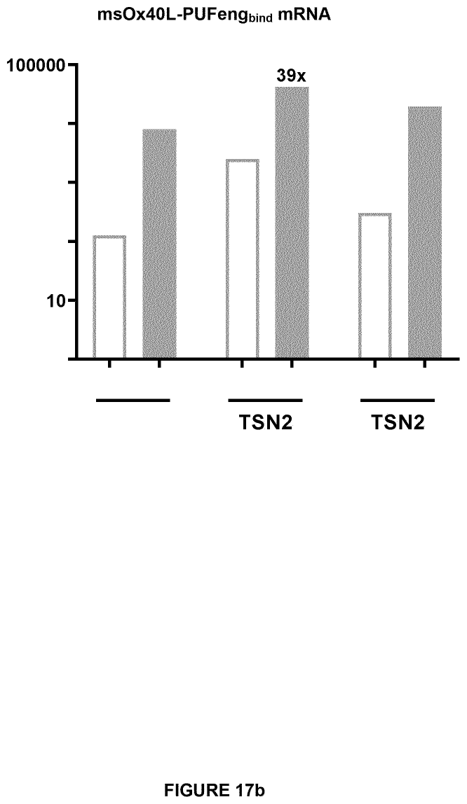

FIGS. 17a and 17b demonstrate that both TSPAN2 and TSPAN3 have the ability to load more NanoLuc mRNA (FIG. 17a) and msO×40L mRNA (FIG. 17b) than the classical EV scaffold protein CD63. When using TSPAN2 as the scaffold protein 8715 copies of NanoLuc mRNA and 41042 copies of O×40L mRNA were loaded and when using TSPAN3 as the scaffold protein 4473 copies of NanoLuc mRNA and 19231 copies of O×40L mRNA were loaded, as compared to 3165 and 7902 copies of NanoLuc mRNA and O×40L mRNA respectively when CD63 was used as the scaffold protein. Interestingly, the data also indicate that passive loading of mRNA (occurring independently of the PUF mRNA binding domain) was also increased when TSPAN2 or TSPAN3 were expressed, as compared to when CD63 was expressed.

### Example 16—Improved Engineering with TSN2

The inventors wanted to investigate how the EV expression of engineered TSPAN2 (also known as TSN2) constructs compares with the EV expression of constructs comprising classic EV scaffold proteins.

HEK-CTS cells (Thermo Fisher Scientific) were transfected with the following constructs:
CD63-Flag-eGFP (Flag on the C-terminus of CD63 and eGFP on the C-terminus of Flag)
CD63-Z(L2)-Flag-eGFP (Flag on the C-terminus of CD63 and eGFP on the C-terminus of Flag; Z domain displayed on the second loop of CD63)
TSN2-Flag-eGFP (Flag on the C-terminus of TSN2 and eGFP on the C-terminus of Flag)
TSN2-Z(L2)-Flag-eGFP (Flag on the C-terminus of TSN2 and eGFP on the C-terminus of Flag; Z domain displayed on the second loop of TSN2).

Three days post transfection, the Conditioned Media (CM) were collected and spun down at 4000×g for 10 min to remove cells and cellular debris. The supernatants were then concentrated (up to 10-fold) down to approximately 200 μL using spin filters (Proteus X-spinner 2.5 Pack, 300 kDa MWCO, PES membrane, cat. PAL-X-300-96; Generon).

The filtered CM was analysed for eGFP content by EV flow cytometry performed on the NanoAnalyser N30E (NanoFCM).

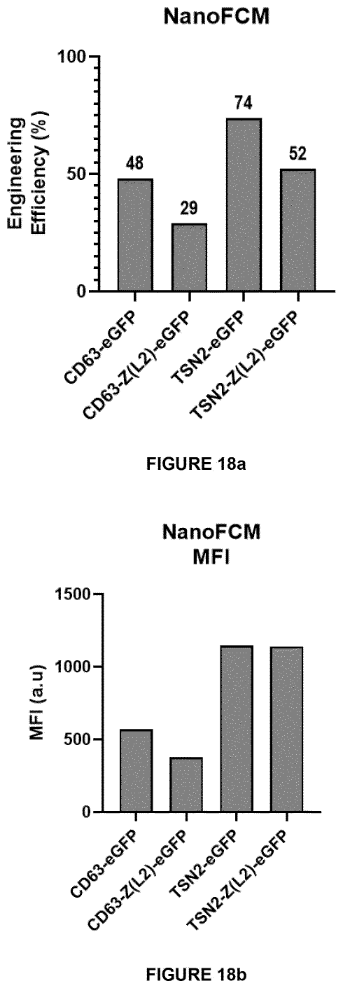

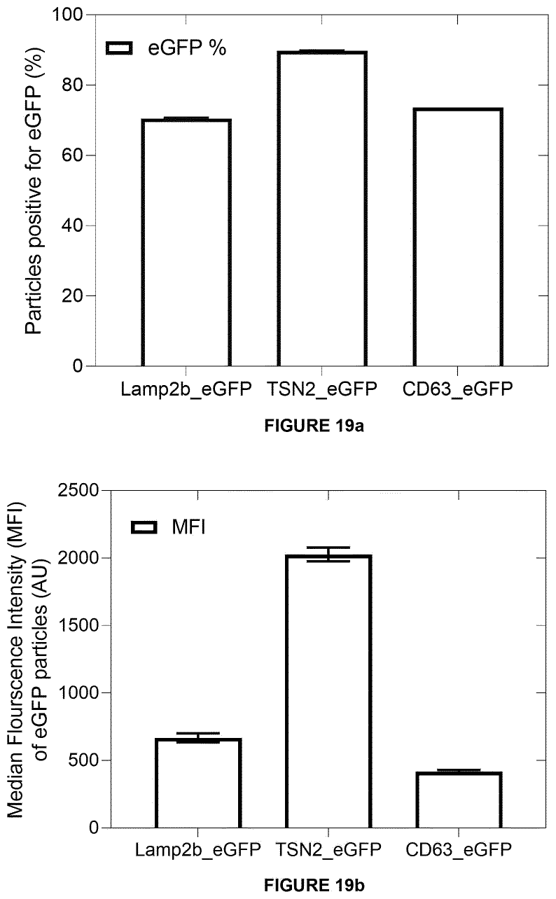

FIG. 18a shows that more of the EVs derived from cells transfected with the TSN2 construct were eGFP positive than the EVs derived from cells transfected with the CD63 construct (compare columns 1 and 3), thus the TSN2 construct results in a more efficient engineering. The insertion of a domain sequence on the second loop reduces the engineering efficiency of both scaffolds (compare columns 1 and 2 and columns 3 and 4), however TSN2 is less affected by this phenomenon.

FIG. 18b shows that the Mean Fluorescence Intensity (MFI) is nearly doubled in EVs derived from cells transfected with the TSN2 construct, as compared to EVs derived from cells transfected with the CD63 construct (compare columns 1 and 3), thus the TSN2 construct results in more scaffold molecules localised to each engineered EV. Whilst insertion of a domain sequence on the second loop of CD63 reduces MFI (compare columns 1 and 2), this is not the case when a domain sequence is engineered on the second loop of TSN2 (compare columns 3 and 4). The data in FIGS. 18a and b show that TSN2 is amenable to significant engineering whilst still retaining an impressively high level of expression of a POI on an EV.

### Example 17—Further Evidence of Improved Engineering with TSN2

The inventors wanted to investigate whether the results described above in Example 16 occurred in a different cell line and with a different modification made to the second loop of the scaffold protein.

EK CTS® VPC.2 cells (Thermo Fisher Scientific) were transfected with the following fusion constructs:
Lamp2b with eGFP engineered on the C-terminus, and a VHH to Transferrin Receptor (TfR) engineered on the N-terminus.
TSN2 with eGFP engineered on the terminus and a VHH to TfR engineered on the loop.
CD63 with with eGFP engineered on the terminus and a VHH to TfR engineered on the loop.

The cells were transfected using plasmid DNA according to the manufacturer's instructions, using FectoPro® (PolyPlus Transfection) as a transfection agent. After 72 hours conditioned media was harvested through differential centrifugation steps starting with 300×g and 700×g for 5 minutes each to remove cells and larger debris. Then at 4000×g for 20 minutes to remove smaller debris. The conditioned media was then ultracentrifuged at 100,000×g for 2 hours to precipitate EVs. The formed pellet was then resuspended in storage buffer and centrifuged at 4000×g to remove any debris that may have formed.

EV flow cytometry was performed on the NanoAnalyser N30E (NanoFCM).

FIG. 19a shows that a larger proportion (over 10% more) of the EVs derived from cells transfected with the TSN2 construct were positive for eGFP, as compared to the EVs derived from cells transfected with either the Lamp2b construct or the CD63 construct.

FIG. 19b shows that there was around a 3-fold improvement in Fluorescence Intensity in the EVs derived from cells transfected with the TSN2 construct, as compared to the EVs derived from cells transfected with the Lamp2b construct. There was around a 4-fold improvement in Fluorescence Intensity in the EVs derived from cells transfected with the TSN2 construct, as compared to the EVs derived from cells transfected with the CD63 construct. This indicates that the EVs derived from the cells transfected with the TSN2 construct have a much higher ligand density than the EVs derived from cells transfected with either the Lamp2b construct or the CD63 construct.

### Example 18—Long Term Stability of Cell Line

In order to test the long term stability of TSPAN2 EVs (both the stability of the expression of the TSPAN2-POI construct alone and also the stability of simultaneous expression of the TSPAN2-POI when co-expressed with a second construct encoding a POI) EV producing cells are manufactured by transduction using lentivirus vectors to express the desired fusion protein(s). Both these producer cells and the EVs produced are then longitudinally examined to observe the stability of the cargo protein expression by Western Blotting, flow cytometry, bioluminescence, etc.

### Example 19—Stability of TSN2 Constructs in EV Producer Cells Over Time

It is a known problem in the field of engineered EV production that expression of constructs that comprise classic tetraspanin EV scaffold proteins are lost from the EV producer cells and the secreted EVs over time. The present inventors went on to investigate the stability of constructs comprising TSPAN2, as compared to those comprising classical tetraspanin EV scaffold CD63, in EV producer cells.

EV producer cells ((HEK293 VPC1.0 CTS (Thermo Fisher Scientific) or CEVEC CAP (CEVEC Pharmaceuticals) cells) were transfected with a CD63-G4S-eGFP construct or a TSN2-G4S-eGFP construct. The G4S linker consists of four glycines and a serine (GlyGlyGlyGlySer), which makes a flexible peptide that connects the eGFP and the scaffold protein. This allows for the presence of the scaffold to be inferred by the presence of the eGFP.

The cells were sorted for high eGFP expression using FACS prior to cryopreservation. Following recovery from revival, antibiotic selection was reintroduced to the culture. The cells were maintained in continuous culture in the presence and absence of antibiotic selection for the duration of the study. Only cells expressing the CD63-G4S-eGFP construct or the TSN2-G4S-eGFP should survive antibiotic selection. The G4S linker consists of four glycines and a serine (GlyGlyGlyGlySer), which makes a flexible peptide that connects the eGFP and the scaffold protein. This allows for the presence of the scaffold to be inferred by the presence of the eGFP

At two timepoints, RNA from cell pellets was extracted (Maxwell® RSC simplyRNA extraction kit; Promega) according to manufacturer's instructions. Timepoint 1 was performed when the cells were at passage 2 and Timepoint 2 when the cells were at passage 13. For the HEK293 cells, the two timepoints were approximately 7 weeks apart and for the CAP cells, the two timepoints were approximately 6 weeks apart.

30 ng of RNA was used for cDNA synthesis using a High-Capacity cDNA synthesis kit (Thermo Fisher Scientific) with the random primers being replaced by 15mer oligoDT according to manufacturer's instructions. The cDNA was evaluated in triplicate using PowerUp Sybergreen PCR. The final cycle threshold (CT) values for GAPDH and eGFP were used to calculate the AACT to determine the absolute change in mRNA expression between eGFP and GAPDH over the period of the study.

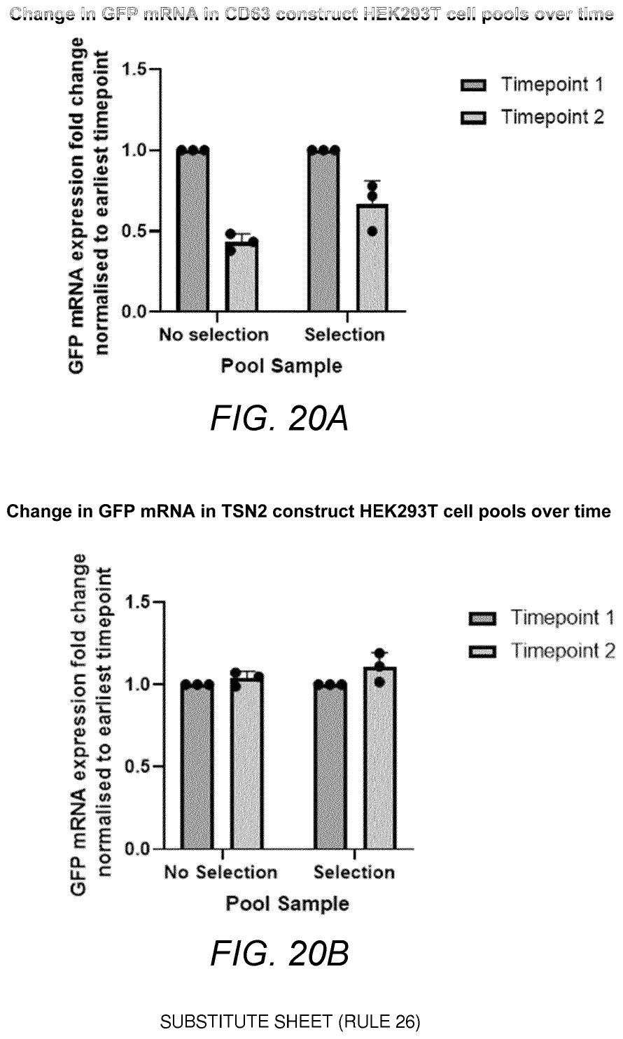

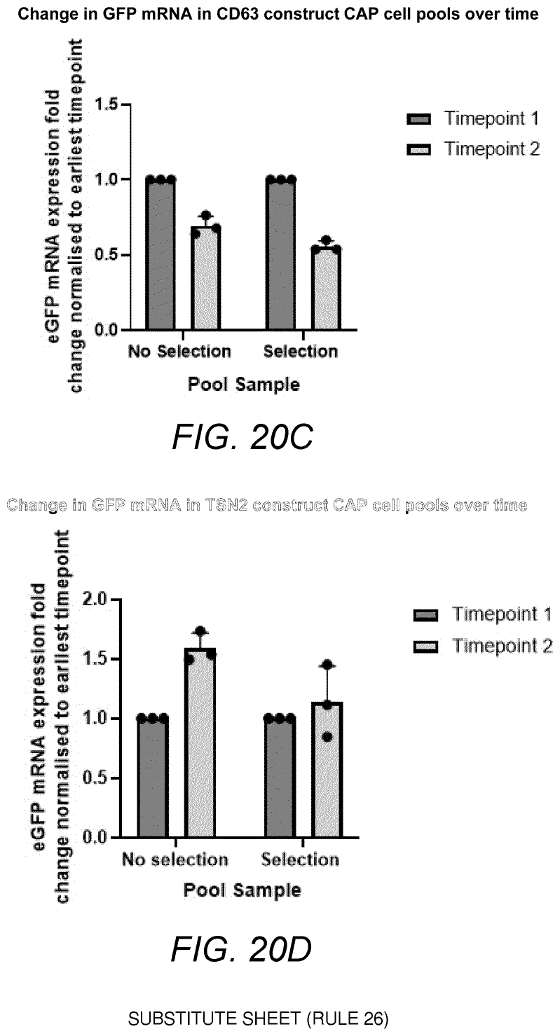

FIG. 20a shows that expression of the CD63 construct was lost from the HEK293T cells over time. Surprisingly, despite also being a tetraspanin, FIG. 20b demonstrates that the TSN2 construct continued to be expressed at a comparable level by the HEK293T cells over time.

FIGS. 20c and 20d show the results observed with CAP cells. In this case, expression of the CD63 construct was lost over time (FIG. 20c), but expression of the TSN2 construct increased over time (FIG. 20d).

Hence, in both HEK and CAP cells, a clear improvement in the stability of eGFP mRNA expression is observed, when the eGFP is expressed in a construct where it is fused to TSN2, rather than CD63. Hence, use of TSN2 as a scaffold protein overcomes the problem of reduced expression over time, which is observed when CD63 is used as a scaffold.

### Example 20—Stability of Engineered EV Production by EV Producer Cells Expressing TSN2 Constructs Over Time

Given the positive results in Example 19, the present inventors went on to investigate the expression of eGFP in EVs secreted by the cells transfected with the TSPAN2 construct, as compared to cells transfected with the CD63 construct.

In these experiments, conditioned media was collected from the EV producer cells (HEK293 VPC1.0 CTS or CEVEC CAP cells, as before) at two time-points. Timepoint 1 was performed when the cells were at passage 2 and Timepoint 2 when the cells were at passage 13. For the HEK293 cells, the two timepoints were approximately 7 weeks apart and for the CAP cells, the two timepoints were approximately 6 weeks apart.

To isolate EVs, 20 mL of conditioned media was centrifuged at 300×g for 5 min and the resulting supernatant was centrifuged at 4000×g and concentrated to 1 mL using Amicon MWCO columns with 100,000 kDa molecular weight limit.

A Pierce Micro BCA assay (Thermo Fisher Scientific) was performed according to manufacturer's instructions and 20 μg of protein was evaluated using Western blot under reducing conditions. Reduced protein was separated using Bolt 4-12% Bis-Tris Gels (Thermo Fisher Scientific) at 200 V for 35 min in 1×MES Running Buffer. Protein was transferred onto a nitrocellulose membrane using an iBlot 2 Membrane prior to blocking treatment with Intercept TBS Blocking Buffer for 1 hour at room temperature. Blocked membranes were probed for eGFP (Chromotek clone 3H9) and Syntenin (Abcam EPR8102). Densitometry analysis was performed by normalising the corresponding syntenin band for the relevant eGFP band at 50 kDa.

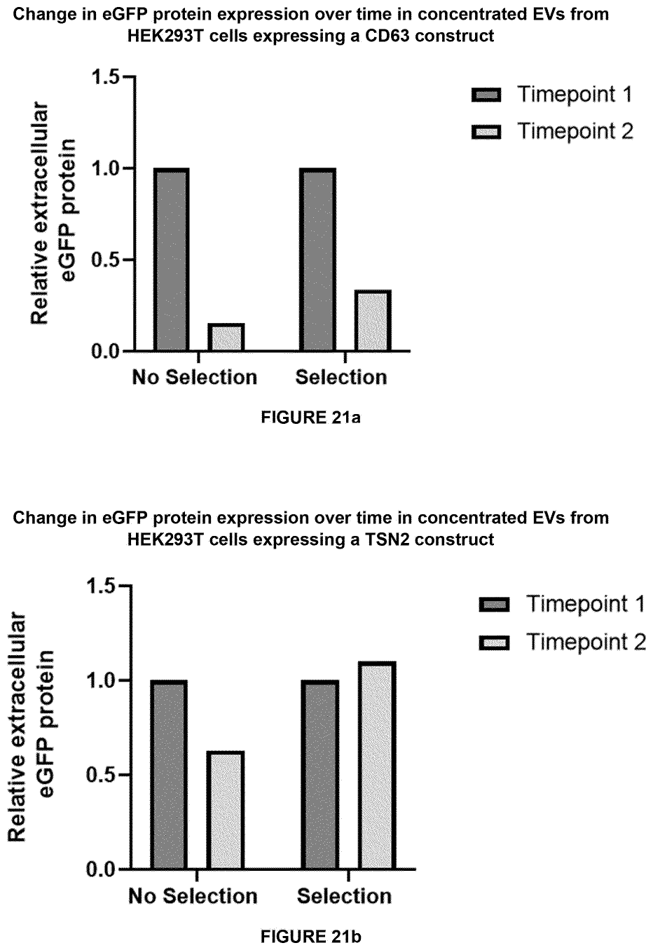

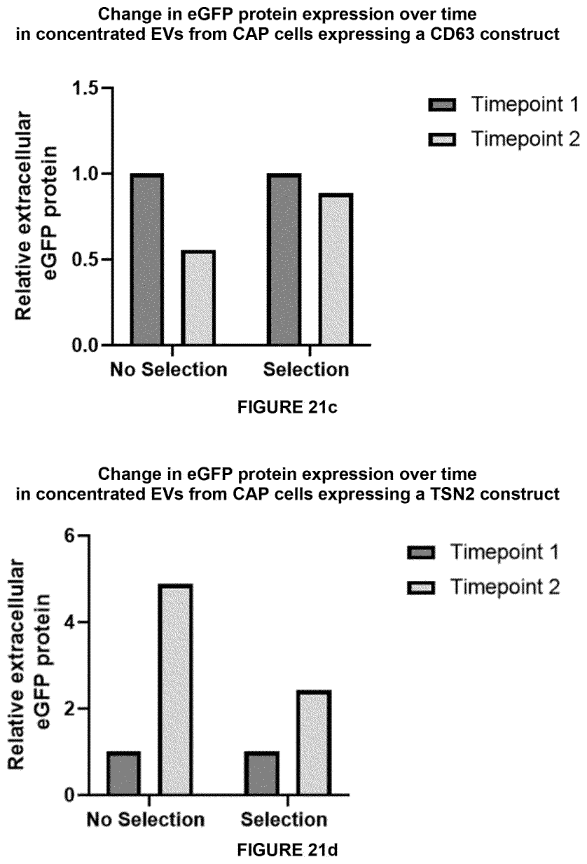

FIG. 21a shows that expression of eGFP in EVs secreted by HEK293T cells that express the CD63 construct was lost over time. FIG. 21b shows that the expression of eGFP in EVs secreted by HEK293T cells that express the TSN2 construct was maintained in cells under antibiotic selection Thus, in a cell population where all the cells express the TSN2-G4S-eGFP construct, the amount of engineered EVs produced remains constant over time. Even in the absence of antibiotic selection, where the cell population comprises a mixed population, with only a proportion of cells expressing the TSN2-G4S-eGFP construct, the relative expression of eGFP in EVs secreted remained above 0.5 at the later timepoint, thus was much improved as compared with the results observed when the CD63-G4S-eGFP construct was expressed.

FIGS. 21c and 21d show the results observed with CAP cells. In this case, expression of eGFP in EVs from CAP cells that express the CD63 construct was also lost over time (FIG. 21c), but expression of eGFP in EVs from CAP cells that express the TSN2 construct increased over time (FIG. 21d).

Hence, in EVs derived from both HEK and CAP cells, a clear improvement in the stability of eGFP expression is observed, when the eGFP is expressed in a construct where it is fused to TSN2, rather than CD63. Hence, use of TSN2 as a scaffold protein helps to overcome the problem of reduced production of engineered EVs over time, which is observed when CD63 is used as a scaffold.

SEQUENCE LISTING

SEQ ID NO: 1 (TSPAN2 protein)
MGRFRGGLRCIKYLLLGFNLLFWLAGSAVIAFGLWFRFGGAIKELSSEDKSPEYFYVGLYVLVGAGAL
MMAVGFFGCCGAMRESQCVLGSFFTCLLVIFAAEVTTGVFAFIGKGVAIRHVQTMYEEAYNDYLKDR
GKGNGTLITFHSTFQCCGKESSEQVQPTCPKELLGHKNCIDEIETIISVKLQLIGIVGIGIAGLTIFGMIFS
MVLCCAIRNSRDVI
SEQ ID NO: 2 (TSPAN2 nucleic acid)
ATGGGCCGGTTTAGGGGCGGTCTGAGATGTATCAAGTACCTCCTCCTTGGTTTCAACCTCCTGTT
TTGGTTGGCCGGAAGCGCTGTGATCGCCTTCGGATTGTGGTTTAGATTCGGAGGCGCAATCAAG
GAACTGTCATCTGAGGACAAGTCACCAGAGTACTTTTACGTGGGGCTCTATGTACTCGTGGGAG
CCGGGGCCCTGATGATGGCCGTGGGTTTCTTCGGGTGTTGCGGAGCAATGAGGGAAAGCCAGT
GCGTGCTTGGGTCTTTCTTTACTTGTCTGCTGGTCATCTTTGCCGCCGAGGTGACTACTGGTGTC
TTTGCATTCATCGGAAAGGGCGTCGCTATCAGGCACGTGCAGACCATGTATGAAGAAGCCTATA
ACGACTATCTCAAGGACCGGGGAAAGGGTAACGGCACCCTTATCACATTTCACTCTACTTTTCAG
TGCTGTGGCAAAGAATCAAGTGAGCAGGTCCAACCCACTTGTCCCAAAGAACTGCTCGGCCATA
AGAACTGCATCGACGAAATCGAGACTATCATCTCCGTGAAGTTGCAGCTCATAGGGATCGTAGG
CATCGGTATTGCTGGTTTAACTATCTTCGGTATGATCTTCAGCATGGTACTGTGTTGTGCTATTAG
AAATAGCCGAGATGTTATA
SEQ ID NO: 3 (TSPAN3 protein)
MGQCGITSSKTVLVFLNLIFWGAAGILCYVGAYVFITYDDYDHFFEDVYTLIPAVVIIAVGALLFIIGLIGC
CATIRESRCGLATFVIILLLVFVTEVVVVVLGYVYRAKVENEVDRSIQKVYKTYNGTNPDAASRAIDYVQ
RQLHCCGIHNYSDWENTDWFKETKNQSVPLSCCRETASNCNGSLAHPSDLYAEGCEALVVKKLQEI
MMHVIWAALAFAAIQLLGMLCACIVLCRRSRDPAYELLITGGTYA
SEQ ID NO: 4 (TSPAN3 nucleic acid)
ATGGGGCAGTGTGGGATCACTAGTAGCAAAACCGTGCTGGTCTTTCTGAATCTTATCTTTTGGGG
AGCCGCGGGGATCCTGTGCTATGTGGGGGCATACGTGTTCATTACCTACGACGACTATGATCAT
TTCTTTGAAGATGTGTACACTCTCATACCCGCAGTTGTCATTATTGCAGTTGGTGCGCTGCTGTT
CATAATAGGACTCATCGGCTGTTGCGCAACTATTCGAGAGAGCAGGTGTGGCCTGGCTACCTTC
GTAATTATCCTGCTTCTGGTATTCGTGACAGAAGTAGTGGTAGTGGTCCTGGGTTACGTTTACAG
GGCGAAGGTGGAGAATGAAGTTGACCGGTCTATTCAAAAAGTCTACAAGACCTATAATGGGACG
AATCCAGATGCCGCGTCTAGAGCTATTGATTATGTGCAGCGACAGCTGCACTGCTGCGGAATCC
ACAATTATTCCGATTGGGAGAACACAGATTGGTTCAAAGAAACAAAGAACCAATCCGTGCCACTG
TCCTGCTGCAGGGAGACAGCATCCAACTGCAATGGATCACTCGCCCATCCCAGTGACCTCTATG
CCGAGGGCTGTGAGGCCTTAGTGGTCAAGAAACTCCAAGAAATTATGATGCACGTGATCTGGGC
TGCCTTGGCCTTCGCAGCTATCCAACTGCTGGGGATGTTATGTGCGTGTATTGTTCTGTGCCGC
CGCAGCAGGGACCCTGCTTACGAGCTCCTGATTACGGGGGGCACCTATGCA
SEQ ID NO: 5 (TSPAN18 protein)
MEGDCLSCMKYLMFVFNFFIFLGGACLLAIGIWVMVDPTGFREIVAANPLLLTGAYILLAMGGLLFLLG
FLGCCGAVRENKCLLLFFFLFILIIFLAELSAAILAFIFRENLTREFFTKELTKHYQGNNDTDVFSATWNS
VMITFGCCGVNGPEDFKFASVFRLLTLDSEEVPEACCRREPQSRDGVLLSREECLLGRSLFLNKQGC
YTVILNTFETYVYLAGALAIGVLAIELFAMIFAMCLFRGIQ
SEQ ID NO: 6 (TSPAN18 nucleic acid)
ATGGAAGGCGATTGTCTCAGTTGCATGAAGTACCTGATGTTCGTTTTCAATTTTTTTATTTTCCTC
GGGGGGGCGTGTCTGCTGGCCATTGGGATTTGGGTGATGGTAGACCCTACAGGCTTTAGGGAA
ATAGTGGCTGCCAACCCTCTGCTCCTTACTGGAGCCTACATTCTTCTGGCCATGGGCGGGCTGC
TGTTTCTCTTGGGCTTCTTGGGGTGTTGCGGAGCCGTCAGAGAAAATAAGTGTTTGCTGCTTTTC
TTCTTTTTGTTCATACTTATAATATTTCTGGCAGAGCTCAGCGCTGCTATTTTGGCATTTATTTTTA
GGGAAAATCTCACCCGCGAATTTTTCACCAAAGAACTGACCAAACATTATCAGGGCAACAATGAC
ACAGATGTATTTTCTGCAACGTGGAACTCTGTTATGATTACTTTTGGCTGCTGCGGGGTAAATGG
CCCTGAGGACTTCAAATTCGCTTCCGTCTTCCGCCTGCTGACCTTGGACTCAGAGGAGGTACCC
GAAGCCTGTTGTCGCAGGGAACCCCAATCAAGAGACGGCGTGCTGCTGTCTAGGGAAGAGTGC
CTGCTGGGGCGTAGTTTATTCCTGAACAAACAGGGTTGTTATACCGTAATCCTGAATACGTTCGA
AACATACGTTTATCTTGCTGGAGCCCTGGCTATTGGGGTGCTCGCTATTGAGCTGTTCGCAATGA
TATTTGCAATGTGTCTTTTCCGGGGCATCCAG

1. An extracellular vesicle (EV) comprising a fusion protein, wherein the fusion protein comprises TSPAN2 fused to a protein of interest (POI).

2. The EV according to claim 1, wherein the POI is engineered into loop 1, loop 2 or both loops of TSPAN2.

3. The EV according to claim 1 or 2, wherein the POI is fused to the N-terminal domain (NTD) or C-terminal domain (CTD) or other luminal portion of TPSAN2.

4. The EV according to any one of the preceding claims, wherein the fusion protein further comprises:
(i) a release domain capable of cleavage to release the POI;
(ii) a linker or spacer;
(iii) a multimerization domain; and/or
(iv) at least one further POI.

5. The EV according to any one of the preceding claims, wherein the EV further comprises a second POI expressed on a separate construct.

6. The EV according to claim 5, wherein the second POI is present as a fusion protein with an exosomal polypeptide.

7. The EV according to any one of the preceding claims, wherein the POI is a:
(i) therapeutic protein;
(ii) binding protein for a therapeutic agent (optionally an RNA binding protein, viral binding protein, Fc-binding protein or small molecule binding protein);
(iii) endosomal escape moiety;
(iv) targeting moiety;
(v) albumin binding domain; or
(vi) purification moiety.

8. The EV according to claim 7, wherein the therapeutic protein is selected from: enzymes, receptors (optionally decoy receptors), membrane proteins, transporters, cytokines, antigens, neoantigens, immune effector molecules, ribonuclear proteins, nucleic acid binding proteins, antibodies, nanobodies, antibody fragments, antibody-drug conjugates, gene editing proteins (optionally CRISPR-Cas), TALENs and meganucleases.

9. The EV according to any one of the preceding claims, wherein the EV is obtainable by a process comprising further exogenously loading the EV with a therapeutic cargo.

10. The EV according to claim 9, wherein the step of further exogenously loading the EV with a therapeutic cargo is by electroporation, transfection reagent, co-incubation or by contact with a cell penetrating peptide (CPP) or any combination thereof.

11. The EV according to claim 9 or 10, wherein the therapeutic cargo is a protein, nucleic acid, virus, viral genome, antigen or a small molecule.

12. The EV according to claim 11, wherein the nucleic acid cargo is: an RNA molecule, a DNA molecule or a mixmer, optionally mRNA, antisense or splice-switching oligonucleotides, gRNA, siRNA, shRNA, miRNA, dbDNA, plasmid DNA (pDNA), supercoiled or unsupercoiled plasmids or mini-circles.

13. A population of EVs according to claim 5, wherein the TSPAN2-POI fusion protein and the second POI construct are expressed on the same EV.

14. A polypeptide construct comprising TSPAN2 fused to a POI.

15. A polynucleotide construct encoding the polypeptide construct of claim 14.

16. A cell comprising the polypeptide construct according to claim 14 or the polynucleotide construct according to claim 15.

17. The cell according to claim 16, further comprising a second polypeptide construct or polynucleotide construct capable of expressing a POI.

18. A pharmaceutical composition comprising an EV according to any one of claims 1 to 12, the population of EVs according to claim 13, or a cell according to claim 16 and a pharmaceutically acceptable excipient or carrier.

19. A method for producing EVs according to any one of claims 1-12, said method comprising:
(i) introducing into an EV-producing cell a polynucleotide construct encoding a TSPAN2-POI fusion construct; and
(ii) expressing the construct in the EV-producing cell, thereby generating EVs comprising the TSPAN2-POI fusion protein.

20. A method for producing EVs according to claim 5, said method comprising:
(i) introducing into an EV-producing cell a polynucleotide construct encoding a TSPAN2-POI fusion construct;
(ii) introducing into the same EV-producing cell a second polynucleotide construct encoding a second POI (optionally wherein the second POI is present in the form of a fusion protein with an EV protein); and
(iii) expressing both constructs in the EV-producing cell, thereby generating EVs comprising the TSPAN2-POI fusion protein and the second POI.

21. The EV according to any one of claims 1-12, for use in medicine.

22. A method of treatment comprising administering to a patient in need thereof, an effective amount of EVs according to claims 1-12 or the pharmaceutical composition according to claim 18.


SEQUENCE LISTING (6 sequences)

SEQ ID NO: 1 - protein, 221 aa, Homo sapiens
MGRFRGGLRCIKYLLLGFNLLFWLAGSAVIAFGLWFRFGGAIKELSSEDKSPEYFYVGLYVLVGAGALMMAVGFFGCCGAMRESQCVLGSFFTCLLVIFAAEVTTGVFAFIGKGVAIRHVQTMYEEAYNDYLKDRGKGNGTLITFHSTFQCCGKESSEQVQPTCPKELLGHKNCIDEIETIISVKLQLIGIVGIGIAGLTIFGMIFSMVLCCAIRNSRDVI

SEQ ID NO: 2 - DNA, 663 bp, Homo sapiens
  source 1..663: mol_type = genomic DNA
atgggccggtttaggggcggtctgagatgtatcaagtacctcctccttggtttcaacctcctgttttggttggccggaagcgctgtgatcgccttcggattgtggtttagattcggaggcgcaatcaaggaactgtcatctgaggacaagtcaccagagtacttttacgtggggctctatgtactcgtgggagccggggccctgatgatggccgtgggtttcttcgggtgttgcggagcaatgagggaaagccagtgcgtgcttgggtctttctttacttgtctgctggtcatctttgccgccgaggtgactactggtgtctttgcattcatcggaaagggcgtcgctatcaggcacgtgcagaccatgtatgaagaagcctataacgactatctcaaggaccggggaaagggtaacggcacccttatcacatttcactctacttttcagtgctgtggcaaagaatcaagtgagcaggtccaacccacttgtcccaaagaactgctcggccataagaactgcatcgacgaaatcgagactatcatctccgtgaagttgcagctcatagggatcgtaggcatcggtattgctggtttaactatcttcggtatgatcttcagcatggtactgtgttgtgctattagaaatagccgagatgttata

SEQ ID NO: 3 - protein, 253 aa, Homo sapiens
MGQCGITSSKTVLVFLNLIFWGAAGILCYVGAYVFITYDDYDHFFEDVYTLIPAVVIIAVGALLFIIGLIGCCATIRESRCGLATFVIILLLVFVTEVVVVVLGYVYRAKVENEVDRSIQKVYKTYNGTNPDAASRAIDYVQRQLHCCGIHNYSDWENTDWFKETKNQSVPLSCCRETASNCNGSLAHPSDLYAEGCEALVVKKLQEIMMHVIWAALAFAAIQLLGMLCACIVLCRRSRDPAYELLITGGTYA

SEQ ID NO: 4 - DNA, 759 bp, Homo sapiens
  source 1..759: mol_type = genomic DNA
atggggcagtgtgggatcactagtagcaaaaccgtgctggtctttctgaatcttatcttttggggagccgcggggatcctgtgctatgtgggggcatacgtgttcattacctacgacgactatgatcatttctttgaagatgtgtacactctcatacccgcagttgtcattattgcagttggtgcgctgctgttcataataggactcatcggctgttgcgcaactattcgagagagcaggtgtggcctggctaccttcgtaattatcctgcttctggtattcgtgacagaagtagtggtagtggtcctgggttacgtttacagggcgaaggtggagaatgaagttgaccggtctattcaaaaagtctacaagacctataatgggacgaatccagatgccgcgtctagagctattgattatgtgcagcgacagctgcactgctgcggaatccacaattattccgattgggagaacacagattggttcaaagaaacaaagaaccaatccgtgccactgtcctgctgcagggagacagcatccaactgcaatggatcactcgcccatcccagtgacctctatgccgagggctgtgaggccttagtggtcaagaaactccaagaaattatgatgcacgtgatctgggctgccttggccttcgcagctatccaactgctggggatgttatgtgcgtgtattgttctgtgccgccgcagcagggaccctgcttacgagctcctgattacggggggcacctatgca

SEQ ID NO: 5 - protein, 248 aa, Homo sapiens
MEGDCLSCMKYLMFVFNFFIFLGGACLLAIGIWVMVDPTGFREIVAANPLLLTGAYILLAMGGLLFLLGFLGCCGAVRENKCLLLFFFLFILIIFLAELSAAILAFIFRENLTREFFTKELTKHYQGNNDTDVFSATWNSVMITFGCCGVNGPEDFKFASVFRLLTLDSEEVPEACCRREPQSRDGVLLSREECLLGRSLFLNKQGCYTVILNTFETYVYLAGALAIGVLAIELFAMIFAMCLFRGIQ

SEQ ID NO: 6 - DNA, 744 bp, Homo sapiens
  source 1..744: mol_type = genomic DNA
atggaaggcgattgtctcagttgcatgaagtacctgatgttcgttttcaatttttttattttcctcgggggggcgtgtctgctggccattgggatttgggtgatggtagaccctacaggctttagggaaatagtggctgccaaccctctgctccttactggagcctacattcttctggccatgggcgggctgctgtttctcttgggcttcttggggtgttgcggagccgtcagagaaaataagtgtttgctgcttttcttctttttgttcatacttataatatttctggcagagctcagcgctgctattttggcatttatttttagggaaaatctcacccgcgaatttttcaccaaagaactgaccaaacattatcagggcaacaatgacacagatgtattttctgcaacgtggaactctgttatgattacttttggctgctgcggggtaaatggccctgaggacttcaaattcgcttccgtcttccgcctgctgaccttggactcagaggaggtacccgaagcctgttgtcgcagggaaccccaatcaagagacggcgtgctgctgtctagggaagagtgcctgctggggcgtagtttattcctgaacaaacagggttgttataccgtaatcctgaatacgttcgaaacatacgtttatcttgctggagccctggctattggggtgctcgctattgagctgttcgcaatgatatttgcaatgtgtcttttccggggcatccag